SEQUENCE LISTING (5 sequences)

SEQ ID NO: 1 - DNA, 22 bp, Staphylococcus aureus
cattgatcgcaacgttcaattt

SEQ ID NO: 2 - DNA, 21 bp, Staphylococcus aureus
tggtctttctgcattcctgga

SEQ ID NO: 3 - DNA, 29 bp, Staphylococcus aureus
tggaagttagattgggatcatagcgtcat

SEQ ID NO: 4 - DNA, 19 bp, Enterococcus faecium
ctgtgaggtcggttgtgcg

SEQ ID NO: 5 - DNA, 17 bp, Enterococcus faecium
tttggtccacctcgcca

(19) United States
(12) Patent Application Publication
(10) Pub. No.: US 2010/0129878 A1
(43) Pub. Date: May 27, 2010

(54) METHODS FOR NUCLEIC ACID AMPLIFICATION

(76) Inventors: Ranjani V. Parthasarathy, Woodbury, MN (US); Hsi-Chou Liu, Woodbury, MN (US); Wensheng Xia, Woodbury, MN (US); Assumpta Bennaars-Eiden, Coon Rapids, MN (US); Paul N. Holt, Hudson, WI (US); William Bedingham, Woodbury, MN (US); Christopher R. Kokaisel, St. Paul, MN (US); Jeffrey C. Pederson, Minneapolis, MN (US)

Correspondence Address: 3M INNOVATIVE PROPERTIES COMPANY, PO BOX 33427, ST. PAUL, MN, 55133-3427, US

(21) Appl. No.: 12/596,346

(22) PCT Filed: Apr. 25, 2008
(86) PCT No.: PCT/US08/61495

§ 371 Date: Oct. 16, 2009

Related U.S. Application Data
(60) Provisional application No. 60/913,813, filed on Apr. 25, 2007.

(51) Int. Cl. C12P 19/34

(52) U.S. Cl. 435/91.5

(57) ABSTRACT

The present invention provides methods for amplifying nucleic acid after isolating nucleic acid from a sample using functionalized support material.

This application claims the benefit of U.S. Provisional Application No. 60/913,813, filed Apr. 25, 2007, which is incorporated herein by reference in its entirety.

### BACKGROUND

The isolation and purification of nucleic acids (DNA and RNA, for example) from complex matrices such as blood, tissue samples, bacterial cell culture media, and forensic samples is an important process in genetic research, nucleic acid probe diagnostics, forensic DNA testing, and other areas that require amplification of the nucleic acids. A variety of methods of preparing nucleic acids for amplification procedures are known in the art; however, each has its limitations.

While treatment such as boiling, hydrolysis with proteinases, exposure to ultrasonic waves, detergents, or strong bases have been used for the extraction of DNA, alkaline extraction is among the simplest of strategies. For example, U.S. Pat. No. 5,620,852 (Lin et al.) describes an efficient extraction of DNA from whole blood performed with alkaline treatment (e.g., NaOH) at room temperature. U.S. Pat. No. 5,010,183 (Kellogg et al.) describes a centrifugal microfluidics-based platform that uses alkaline lysis for DNA extraction from blood. Another conventional method uses a phenol chloroform extraction.

International Publication No. WO 01/37291 A1 (MagNA Pure) describes the use of magnetic glass particles and an isolation method in which samples are lysed by incubation with a special buffer containing a chaotropic salt and proteinase K. Glass magnetic particles are added and total nucleic acids contained in the sample are bound to their surface. Unbound substances are removed by several washing steps. Finally, purified total nucleic acid is eluted with a low salt buffer at high temperature.

Yet another conventional method involves applying a biological sample to a hydrophobic organic polymeric solid phase to selectively trap nucleic acid and subsequently remove the trapped nucleic acid with a nonionic surfactant. Another method involves treating a hydrophobic organic polymeric material with a nonionic surfactant, washing the surface, and subsequently contacting the treated solid organic polymeric material with a biological sample to reduce the amount of nucleic acid that binds to the organic polymeric solid phase. Although these solid phase methods are effective methods for isolating nucleic acid from biological samples, other methods are needed, particularly methods that are suitable for use in microfluidic devices.

### SUMMARY

The present invention provides methods and kits for the amplification of nucleic acids.

Nucleic acids isolated according to the invention, will be useful, for example, in assays for detection of the presence of a particular nucleic acid in a sample. Such assays are important in the prediction and diagnosis of disease, forensic medicine, epidemiology, and public health. For example, DNA may be subjected to amplification to detect the presence of an infectious virus or a mutant gene in an individual, allowing determination of the probability that the individual will suffer from a disease of infectious or genetic origin. The ability to detect an infectious virus or a mutation in one sample among the hundreds or thousands of samples being screened takes on substantial importance in the early diagnosis or epidemiology of an at-risk population for disease, e.g., the early detection of HIV infection, cancer or susceptibility to cancer, or in the screening of newborns for diseases, where early detection may be instrumental in diagnosis and treatment.

The present invention provides methods and kits for amplifying nucleic acid from a sample that includes nucleic acid (e.g., DNA, RNA, PNA), which may or may not be included within nuclei-containing cells (e.g., white blood cells).

The present invention involves the use of functionalized support material (i.e., functionalized solid phase material), preferably nonspecific functionalized support material. Suitable solid phase materials typically include a solid matrix in the form of particulate material (e.g., particles, beads, microbeads, microspheres) or any other form (e.g., fibrils) that can be introduced into a microfluidic device (e.g., a device with a process array that includes process chambers defining volumes for containing sample material or portions thereof (e.g., nucleic acid-containing material, nucleic acid) and a conduit connecting the process chambers) and transported therethrough. Such solid matrix includes capture sites (e.g., functional groups) attached thereto for attachment to nucleic acid-containing material and nucleic acid. For example, beads can be functionalized with the appropriate groups to capture cells (including spores), viruses, bacterial cells, proteins, nucleic acids, etc. The functionalized support material, in particular beads, microbeads, microspheres, or particles, can be segregated from the sample by compaction through, e.g., centrifugation, and subsequent separation. The functionalized support material, in particular beads, microbeads, microspheres, or particles, can be designed to have the appropriate density and sizes (nanometers to microns) for segregation.

The methods of the present invention can include the use of processing devices that include one or more process arrays with thermal transfer structures (including, for example, a waste chamber) that can be used alone or in conjunction with rotation to transport fluids within a microfluidic system. The thermal transport function can be accomplished by changing the temperature of one or more chambers to create a vacuum to draw fluids (e.g., gases and/or liquids) in selected directions within the process array. Among the potential advantages of such methods (thermopipetting methods) are the ability to move fluids in a direction that is against the direction of gravitational forces and/or centrifugal forces generated by rotating a processing device using the thermal transfer structures. In other words, fluids may be moved against the force of gravity or towards an axis of rotation using the thermally-activated vacuum. As used herein, the term “vacuum” refers to a pressure differential between volumes in a process array large enough to move fluids in a selected direction.

The thermal transfer structure may also be used to control fluid movement within the processing device without the need for physical valve structures that require opening or closing of physical structures to allow for fluid passage. For example, the dimensions, geometry, materials, etc., may be selected such that fluid passage will not typically occur in the absence of a vacuum. One feature that may be used is a conduit that includes a fluid trap as described herein. In such instances, the thermally-activated vacuum provided by a thermal transfer structure can be used to control fluid movement within a process array.

In some embodiments, the thermal transfer structure can include a thermal drive chamber located in an area of the processing device that is remote from the chambers between which fluid is to be transported. The remote thermal drive chamber can be fluidly connected to the chambers between which fluid is to be transported by a conduit formed in the device. One potential advantage of such a structure is that the portion of the processing device heated (or cooled) to create the vacuum may be sufficiently removed from the chambers between which fluid is to be transported such that the analytes in the transported fluids are not significantly heated or cooled.

Thermal transfer structures and methods may also be used to transport multiple discrete volumes (aliquots) of fluids (sequentially and/or simultaneously) into or through a chamber in a process array. Such control over fluid transport can be used for, e.g., washing to remove unwanted materials from a sample, delivery of reagents at selected times and in selected amounts, etc. When used to transfer multiple discrete volumes of fluids, the thermal transfer structures may operate more effectively due to the presence of liquids in the thermal drive chambers where at least a portion of the liquid changes phase to become a gas. Such phase changes may increase the volumetric changes in the resident fluid (e.g., gas and/or liquid) caused by heating and, thus, the resulting vacuum force may also increase as the resident fluid is cooled.

In one embodiment, the present invention provides a method of amplifying nucleic acid, the method comprising: providing a device comprising a process array that comprises process chambers defining volumes for containing sample material or portions thereof (e.g., nucleic acid-containing material, nucleic acid) and a conduit connecting the process chambers; delivering sample material comprising nucleic acid-containing material (e.g., bacteria) to a process chamber of the device; contacting the nucleic acid-containing material with one or more lysing reagents (which can include an alkaline lysis reagent and a surfactant in a lysis buffer, for example) under conditions effective to lyse (which can include neutralizing conditions) at least a portion of the nucleic acid-containing material and release nucleic acid; in the same or a different process chamber, contacting the nucleic acid with a first functionalized support material (preferably, a first nonspecific functionalized support material) under conditions effective to capture at least a portion of the nucleic acid by the first functionalized support material; washing the first functionalized support material with nucleic acid attached thereto multiple times (using multiple aliquots of one or more wash solutions, such as a wash buffer) in the same process chamber (which can be the same chamber in which lysing and/or capturing occurs); transferring the first functionalized support material with nucleic acid attached thereto to a different process chamber through the conduit by rotating the device; before, during, or after transferring the first functionalized support material with nucleic acid attached thereto, contacting it with an amplification reagent (which can be located in the different process chamber or in a separate chamber), wherein the amplification reagent comprises one or more primers and optionally one or more probes (which are preferably present); and providing conditions effective to amplify the nucleic acid to produce amplicons.

In another embodiment, the present invention provides a method of amplifying nucleic acid, the method comprising: providing a device comprising a process array that comprises process chambers defining volumes for containing sample material or portions thereof (e.g., nucleic acid-containing material, nucleic acid), a conduit connecting the process chambers, and a thermal transfer structure comprising a thermal drive chamber (which can be a waste chamber) and resident fluid (gas and/or liquid); delivering sample material comprising nucleic acid-containing material (e.g., bacteria) to a process chamber of the device; contacting the nucleic acid-containing material with one or more lysing reagents (which can include an alkaline lysis reagent and a surfactant in a lysis buffer, for example) under under conditions effective to lyse (which can include neutralizing conditions) at least a portion of the nucleic acid-containing material and release nucleic acid; in the same or a different process chamber, contacting the nucleic acid with a first functionalized support material (preferably, a first nonspecific functionalized support material) under conditions effective to capture at least a portion of the nucleic acid by the first functionalized support material; washing and/or decanting the first functionalized support material with nucleic acid attached thereto using the thermal transfer structure, comprising: optionally adding a wash solution to the process chamber comprising the first functionalized support material with nucleic acid attached thereto; passing a first portion of the resident fluid (typically, gas) through a thermal transfer conduit into the process chamber comprising the first functionalized support material with nucleic acid attached thereto by heating at least a portion of the resident fluid in the thermal drive chamber (typically, a waste chamber), wherein the thermal transfer conduit connects the thermal drive chamber to the process chamber comprising the first functionalized support material with nucleic acid attached thereto, and wherein the volume of the resident fluid within the thermal transfer structure increases to force the first portion of the resident fluid into the process chamber comprising the first functionalized support material with nucleic acid attached thereto; and subsequently cooling the heated resident fluid in the thermal transfer structure, wherein the volume of the resident fluid within the thermal transfer structure decreases such that at least a portion of the liquid present in the process chamber comprising the first functionalized support material with nucleic acid attached thereto is drawn into the thermal drive chamber through the thermal transfer conduit; transferring the first functionalized support material with nucleic acid attached thereto to a different process chamber through the conduit by rotating the device; before, during, or after transferring the first functionalized support material with nucleic acid attached thereto, contacting it with an amplification reagent (which can be located in the different process chamber or in a separate chamber), wherein the amplification reagent comprises one or more primers and optionally one or more probes (which are preferably present); and providing conditions effective to amplify the nucleic acid to produce amplicons.

In yet another embodiment, the present invention provides a method of amplifying nucleic acid, the method comprising: providing a device comprising a process array that comprises process chambers defining volumes for containing sample material or portions thereof (e.g., nucleic acid-containing material, nucleic acid) and a conduit connecting the process chambers; delivering sample material comprising nucleic acid-containing material (e.g., bacteria) to a process chamber of the device; contacting the nucleic acid-containing material with one or more lysing reagents (which can include an alkaline lysis reagent and a surfactant in a lysis buffer, for example) under conditions effective to lyse (which can include neutralizing conditions) at least a portion of the nucleic acid-containing material and release nucleic acid; in the same or a different process chamber, contacting the nucleic acid with a first nonspecific functionalized support material under conditions effective to capture at least a portion of the nucleic acid by the first nonspecific functionalized support material, wherein the first nonspecific functionalized support material contacting the nucleic acid comprises immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; wherein M is selected from the group consisting of zirconium, gallium, iron, aluminum, scandium, titanium, vanadium, yttrium, and a lanthanide; y is an integer from 3 to 6; and x is 1 or 2; washing the first nonspecific functionalized support material with nucleic acid attached thereto; transferring the first nonspecific functionalized support material with nucleic acid attached thereto to a different process chamber through the conduit by rotating the device; before, during, or after transferring the first nonspecific functionalized support material with nucleic acid attached thereto, contacting it with an amplification reagent (which can be located in the different process chamber or in a separate chamber), wherein the amplification reagent comprises one or more primers and optionally one or more probes (which are preferably present); and providing conditions effective to amplify the nucleic acid to produce amplicons.

In certain aspects of the above-identified three embodiments, providing conditions effective to amplify the nucleic acid to produce amplicons comprises contacting the nucleic acid and the amplification reagent with one or more amplification enzymes under conditions effective to amplify the nucleic acid to produce amplicons. Such conditions can include the types and amounts of reagents, the time, the temperature, etc.

In alternative aspects of these embodiments, transferring the first functionalized support material (preferably, first nonspecific functionalized support material) with nucleic acid attached thereto to a different process chamber and contacting it with an amplification reagent before, during, or after the transfer, comprises: transferring the first functionalized support material (preferably, the first nonspecific functionalized support material) with nucleic acid attached thereto to a different process chamber through the conduit by rotating the device; wherein the nucleic acid contacts an amplification reagent located in the different process chamber; wherein the amplification reagent comprises one or more primers, optionally one or more probes (which are preferably present), and one or more amplification enzymes; and wherein the one or more amplification enzymes are thermally stable at the temperature effective to release and/or denature (preferably, release and denature) the nucleic acid; and prior to (or while) providing conditions effective to amplify the nucleic acid, the method further comprises: heating the first functionalized support material (preferably, the first nonspecific functionalized support material) with nucleic acid attached thereto at a temperature effective to release and/or denature (preferably, release and denature) at least a portion of the nucleic acid in the presence of the amplification reagent.

In certain aspects of these embodiments, prior to delivering sample material to a process chamber of the device, the method comprises: providing sample material comprising nucleic acid-containing material (e.g., bacteria); and contacting the nucleic acid-containing material with a second functionalized support material (preferably, second nonspecific functionalized support material), which may be the same or different than the first functionalized support material used to capture at least a portion of the nucleic acid, under conditions effective to capture at least a portion of the nucleic acid-containing material by the second functionalized support material; and delivering sample material to a process chamber of the device comprises delivering the second functionalized support material and captured nucleic acid-containing material to a process chamber of the device.

In alternative aspects of these embodiments, after delivering sample material to a process chamber of the device, the method comprises: contacting the nucleic acid-containing material (e.g., bacteria) with a second functionalized support material (preferably, second nonspecific functionalized support material), which may be the same or different than the first functionalized support material used to capture at least a portion of the nucleic acid, under conditions effective to capture at least a portion of the nucleic acid-containing material by the second functionalized support material in the process chamber; and transferring the second functionalized support material and captured nucleic acid-containing material to a different process chamber; and contacting the nucleic acid-containing material with one or more lysing reagents comprises contacting the nucleic acid-containing material attached to the second functionalized support material with one or more lysing reagents under conditions effective to lyse at least a portion of the nucleic acid-containing material and release nucleic acid.

Preferably, the functionalized support material (preferably, nonspecific functionalized support material) that is used to capture the nucleic acid (the “first” functionalized support material mentioned above) is different than the functionalized support material (preferably, nonspecific functionalized support material) used to capture the nucleic acid-containing material (e.g., bacteria) (the “second” functionalized support material mentioned above). Different functionalized support material (preferably, nonspecific functionalized support material) is desired if lysis is inefficient and/or steric hinderance prevents binding of released nucleic acid on to the same functionalized support material (preferably, nonspecific functionalized support material).

As stated above, suitable functionalized support material includes solid phase materials, which typically include a solid matrix in the form of particulate material (e.g., particles, microspheres, beads, microbeads) or any other form (e.g., fibrils) that can be introduced into, and transported through, a microfluidic device. Preferred materials include particulate material.

Preferably, the functionalized support material, for any of the embodiments described herein, is nonspecific functionalized support material that includes immobilized-metal support material including a substrate (preferably, particulate material) having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; wherein M is selected from the group consisting of zirconium, gallium, iron, aluminum, scandium, titanium, vanadium, yttrium, and a lanthanide; y is an integer from 3 to 6; and x is 1 or 2.

In certain embodiments of the methods of the present invention, contacting the nucleic acid-containing material with one or more lysing reagents and contacting the released nucleic acid with functionalized support material occur in the same chamber (typically the same process chamber); whereas in other embodiments, these contacting steps can occur in different chambers (e.g., different process and/or mixing chambers).

In the methods of the present invention, the lysing reagent is included within a lysis buffer, which can include two or more lysing reagents. The lysing reagent preferably includes an enzyme, a base, a surfactant, or combinations thereof. If the lysis buffer includes a basic lysing reagent, the method typically further includes contacting the nucleic acid, optionally attached to functionalized support material (preferably, nonspecific functionalized support material), with a neutralization reagent in a subsequent step.

For certain preferred embodiments, the nucleic acid-containing material includes bacterial cells, and the method includes: delivering sample material including bacterial cells to a process chamber; contacting the bacterial cells with functionalized support material (preferably, nonspecific functionalized support material) under conditions effective to capture at least a portion of the bacterial cells by the functionalized support material in the process chamber; transferring the functionalized support material and captured bacterial cells to a different process chamber; and contacting the bacterial cells attached to the functionalized support material with one or more lysing reagents (which can be included in a lysis buffer) including an enzyme under conditions effective to lyse at least a portion of the bacterial cells, release nucleic acid, and capture at least a portion of the nucleic acid by the same functionalized support material to which the bacterial cells were attached.

For certain preferred embodiments, the nucleic acid-containing material includes bacterial cells, and the method includes: delivering sample material including bacterial cells to a process chamber; contacting the bacterial cells with functionalized support material (preferably, nonspecific functionalized support material) under conditions effective to capture at least a portion of the bacterial cells by the functionalized support material in the process chamber; transferring the functionalized support material and captured bacterial cells to a different process chamber; and contacting the bacterial cells attached to the functionalized support material, with one or more lysing reagents (which can be included in a lysis buffer) under conditions effective to lyse at least a portion of the bacterial cells and release nucleic acid; in a different process chamber, contacting the released nucleic acid with functionalized support material, which may be the same or different than the functionalized support material optionally used to capture the bacterial cells, under conditions effective to capture at least a portion of the nucleic acid by the same or different functionalized support material. Preferably, at least a portion of the released nucleic acid is captured by different functionalized support material (preferably, nonspecific functionalized support material) than used to capture the bacterial cells.

If desired, the methods described herein can involve lysis outside of the device. Examples of such embodiments follow.

In another embodiment, the present invention provides a method of amplifying nucleic acid, the method comprising: providing a device comprising a process array that comprises process chambers defining volumes for containing sample material or portions thereof (e.g., nucleic acid-containing material, nucleic acid) and a conduit connecting the process chambers; providing sample material comprising lysed nucleic acid-containing material that includes nucleic acid; contacting the lysed nucleic acid-containing material with a functionalized support material (preferably, nonspecific functionalized support material) under conditions effective to capture at least a portion of the nucleic acid by the functionalized support material; prior to or after contacting the lysed nucleic acid-containing material with the functionalized support material, delivering the nucleic acid, optionally attached to the functionalized support material, to a process chamber of the device; washing the functionalized support material with nucleic acid attached thereto multiple times in the same process chamber using multiple aliquots of one or more wash solutions; transferring the functionalized support material with nucleic acid attached thereto to a different process chamber through the conduit by rotating the device; before, during, or after transferring the functionalized support material with nucleic acid attached thereto, contacting it with an amplification reagent (which can be located in the different process chamber or in a separate chamber), wherein the amplification reagent comprises one or more primers and optionally one or more probes (which are preferably present); and providing conditions effective to amplify the nucleic acid to produce amplicons.

In yet another embodiment, the present invention provides a method comprising: providing a device comprising a process array that comprises process chambers defining volumes for containing sample material or portions thereof (e.g., nucleic acid-containing material, nucleic acid), a conduit connecting the process chambers, and a thermal transfer structure comprising a thermal drive chamber (which can be a waste chamber) and resident fluid (gas and/or liquid); providing sample material comprising lysed nucleic acid-containing material that includes nucleic acid; contacting the lysed nucleic acid-containing material with a functionalized support material (preferably, a nonspecific functionalized support material) under conditions effective to capture at least a portion of the nucleic acid by the functionalized support material; prior to or after contacting the lysed nucleic acid-containing material with the functionalized support material, delivering the nucleic acid, optionally attached to the functionalized support material, to a process chamber of the device; washing and/or decanting the functionalized support material with nucleic acid attached thereto using the thermal transfer structure, comprising: optionally adding a wash solution to the process chamber comprising the first functionalized support material with nucleic acid attached thereto; passing a first portion of the resident fluid (typically, gas) through a thermal transfer conduit into the process chamber comprising the functionalized support material with nucleic acid attached thereto by heating at least a portion of the resident fluid in the thermal drive chamber (typically, a waste chamber), wherein the thermal transfer conduit connects the thermal drive chamber to the process chamber comprising the functionalized support material with nucleic acid attached thereto, and wherein the volume of the resident fluid within the thermal transfer structure increases to force the first portion of the resident fluid into the process chamber comprising the functionalized support material with nucleic acid attached thereto; and subsequently cooling the heated resident fluid in the thermal transfer structure, wherein the volume of the resident fluid within the thermal transfer structure decreases such that at least a portion of the liquid present in the process chamber comprising the first functionalized support material with nucleic acid attached thereto is drawn into the thermal drive chamber through the thermal transfer conduit; transferring the functionalized support material with nucleic acid attached thereto to a different process chamber through the conduit by rotating the device; before, during, or after transferring the functionalized support material with nucleic acid attached thereto, contacting it with an amplification reagent (which can be located in the different process chamber or in a separate chamber), wherein the amplification reagent comprises one or more primers and optionally one or more probes (which are preferably present); and providing conditions effective to amplify the nucleic acid to produce amplicons.

In still another embodiment, the present invention provides a method of amplifying nucleic acid, the method comprising: providing a device comprising a process array that comprises process chambers defining volumes for containing sample material or portions thereof (e.g., nucleic acid-containing material, nucleic acid) and a conduit connecting the process chambers; providing sample material comprising lysed nucleic acid-containing material that includes nucleic acid; contacting the lysed nucleic acid-containing material with a nonspecific functionalized support material under conditions effective to capture at least a portion of the nucleic acid by the nonspecific functionalized support material, wherein the nonspecific functionalized support material comprises immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; wherein M is selected from the group consisting of zirconium, gallium, iron, aluminum, scandium, titanium, vanadium, yttrium, and a lanthanide; y is an integer from 3 to 6; and x is 1 or 2; prior to or after contacting the lysed nucleic acid-containing material with the nonspecific functionalized support material, delivering the nucleic acid, optionally attached to the nonspecific functionalized support material, to a process chamber of the device; washing the nonspecific functionalized support material with nucleic acid attached thereto on the device; transferring the nonspecific functionalized support material with nucleic acid attached thereto to a different process chamber through the conduit by rotating the device; before, during, or after transferring the nonspecific functionalized support material with nucleic acid attached thereto, contacting it with an amplification reagent (which can be located in the different process chamber or in a separate chamber), wherein the amplification reagent comprises one or more primers and optionally one or more probes (which are preferably present); and providing conditions effective to amplify the nucleic acid to produce amplicons.

In certain aspects of the above-identified three embodiments, providing conditions effective to amplify the nucleic acid to produce amplicons comprises contacting the nucleic acid and the amplification reagent with one or more amplification enzymes under conditions effective to amplify the nucleic acid to produce amplicons. Such conditions can include the types and amounts of reagents, the time, the temperature, etc.

In alternative aspects of these embodiment, transferring the functionalized support material (preferably, nonspecific functionalized support material) with nucleic acid attached thereto to a different process chamber and contacting it with an amplification reagent comprises: transferring the functionalized support material (preferably, nonspecific functionalized support material) with nucleic acid attached thereto to a different process chamber through the conduit by rotating the device; wherein the nucleic acid contacts an amplification reagent located in the different process chamber; wherein the amplification reagent comprises one or more primers, optionally one or more probes (which are preferably present), and one or more amplification enzymes; and wherein the one or more amplification enzymes are thermally stable at the temperature effective to release and/or denature (preferably, release and denature) the nucleic acid; and prior to (or while) providing conditions effective to amplify the nucleic acid, the method further comprises: heating the functionalized support material (preferably, nonspecific functionalized support material) with nucleic acid attached thereto at a temperature effective to release and/or denature (preferably, release and denature) at least a portion of the nucleic acid in the presence of the amplification reagent.

In certain aspects of these embodiments, delivering the nucleic acid to a process chamber of the device comprises delivering functionalized support material (preferably, nonspecific functionalized support material) and captured nucleic acid to a process chamber of the device.

In alternative aspects of these embodiments, delivering the nucleic acid to a process chamber of the device comprises delivering sample material comprising lysed nucleic acid-containing material that includes nucleic acid to a process chamber of the device prior to capturing at least a portion of the nucleic acid by the functionalized support material (preferably, nonspecific functionalized support material).

### DEFINITIONS

“Nucleic acid” shall have the meaning known in the art and refers to DNA (e.g., genomic DNA, cDNA, or plasmid DNA), RNA (e.g., mRNA, tRNA, or rRNA), and PNA. It can be in a wide variety of forms, including, without limitation, double-stranded or single-stranded configurations, circular form, plasmids, relatively short oligonucleotides, peptide nucleic acids also called PNA's (as described in Nielsen et al., Chem. Soc. Rev., 26, 73-78 (1997)), and the like. The nucleic acid can be genomic DNA, which can include an entire chromosome or a portion of a chromosome. The DNA can include coding (e.g., for coding mRNA, tRNA, and/or rRNA) and/or noncoding sequences (e.g., centromeres, telomeres, intergenic regions, introns, transposons, and/or microsatellite sequences). The nucleic acid can include any of the naturally occurring nucleotides as well as artificial or chemically modified nucleotides, mutated nucleotides, etc. The nucleic acid can include a non-nucleic acid component, e.g., peptides (as in PNA's), labels (radioactive isotopes or fluorescent markers), and the like.

“Nucleic acid-containing material” refers to a source of nucleic acid such as a cell (e.g., white blood cells, enucleated red blood cells, spores), a nuclei, a virus, a fungus, or any other composition that houses a structure that includes nucleic acid (e.g., plasmid, cosmid, or viroid, archeobacteriae). The cells can be prokaryotic (e.g., gram positive or gram negative bacteria) or eukaryotic (e.g., blood cell or tissue cell). If the nucleic acid-containing material is a virus, it can include an RNA or a DNA genome; it can be virulent, attenuated, or noninfectious; and it can infect prokaryotic or eukaryotic cells. The nucleic acid-containing material can be naturally occurring, artificially modified, or artificially created.

“Support material” or “solid support material” or “solid phase material” refers to an inorganic and/or organic material, such as a polymer made of repeating units, which may be the same or different, of organic and/or inorganic compounds of natural and/or synthetic origin. This includes homopolymers and heteropolymers (e.g., copolymers, terpolymers, tetrapolymers, etc., which may be random or block, for example). This term includes fibrous or particulate forms (e.g., beads, microbeads, microspheres, or particles) of a material, which can be readily prepared by methods well-known in the art and which can be introduced into and transported through a microfluidic device.

The term “functionalized” in the context of “functionalized support material” refers to material that includes capture sites, i.e., sites on the support material to which a nucleic acid-containing material and/or nucleic acid adheres. Typically, the capture sites include functional groups or molecules that are either covalently attached or otherwise attached (e.g., hydrophobically attached) to the support material.

The term “nonspecific” in the context of “nonspecific functionalized support material” refers to material that does not capture specifically any one particular nucleic acid-containing material or one any particular nucleic acid relative to all others. For example, although certain nonspecific functionalized support material may capture bacteria as opposed to fungi, there is no specific capture of any one species of bacteria. This is because the interaction generally results from various mechanisms, such as the attraction between a positively charged bead surface and a negative charged cell surface. There are no functional groups or molecules associated with the functionalized support material that are specific for a particular species of microorganism, or a particular nucleic acid, etc.

“Capture” or “attach” or “bind” (or variations thereof) as they refer to the nucleic acid-containing material and/or nucleic acid with respect to the functionalized support material (preferably, nonspecific functionalized support material) refer to reversible retention via a wide variety of mechanisms, including weak forces such as Van der Waals interactions, electrostatic interactions, affinity binding, or physical trapping. The use of these terms does not imply a mechanism of action, and includes adsorptive and absorptive mechanisms.

“Surfactant” refers to a substance that lowers the surface or interfacial tension of the medium in which it is dissolved. It is used interchangeably herein with “detergent.”

“Microfluidic” refers to a device with one or more fluid passages, chambers, or conduits that have at least one internal cross-sectional dimension, e.g., depth, width, length, diameter, etc., that is less than 500 μm, and typically between 0.1 μm and 500 μm. In the devices used in the present invention, the microscale channels or chambers preferably have at least one cross-sectional dimension between 0.1 μm and 200 μm, more preferably between 0.1 μm and 100 μm, and often between 1 μm and 20 μm. Typically, a microfluidic device includes a plurality of chambers (process chambers, separation chambers, mixing chambers, waste chambers, diluting reagent chambers, amplification reaction chambers, loading chambers, and the like), each of the chambers defining a volume for containing a sample; and at least one distribution channel connecting the plurality of chambers of the array; wherein at least one of the chambers within the array can include a solid phase material (thereby often being referred to as a separation chamber) and/or at least one of the process chambers within the array can include a lysing reagent (thereby often being referred to as a mixing chamber), for example.

The terms “comprises” and variations thereof do not have a limiting meaning where these terms appear in the description and claims.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, “a,” “an,” “the,” “at least one,” and “one or more” are used interchangeably. Thus, for example, a process chamber that comprises “an” amplification enzyme can be interpreted to mean that the process chamber includes “one or more” amplification enzymes.

The term “and/or” means one or all of the listed elements or a combination of any two or more of the listed elements (e.g., (isolated) nucleic acid and/or cells means (isolated) nucleic acid, cells, or both nucleic acid and cells).

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list. Furthermore, various embodiments are described in which the various elements of each embodiment could be used in other embodiments, even though not specifically described.

### BRIEF DESCRIPTION OF THE DRAWINGS

FIG. 1 is a top view of an exemplary device according to the present invention with two separate chambers.

FIGS. 2-4 are plan views of exemplary process arrays according to the present invention.

FIG. 5 shows amplification curves of TGFβ RT-PCR amplifications on a MX3005P (Stratagene) instrument.

FIG. 6 shows amplification curves of TGFβ RT-PCR amplifications on a 7900HT (ABI) instrument.

### DETAILED DESCRIPTION OF ILLUSTRATIVE EMBODIMENTS

The present invention provides various methods, devices, and kits for capturing nucleic acid from a sample, typically a biological sample, using a flow through receptacle (typically in a microfluidic format) followed by amplification. The present invention provides methods, devices, and kits for capturing nucleic acid from a sample that includes nucleic acid (e.g., DNA, RNA, PNA), which may or may not be included within nuclei-containing cells (e.g., white blood cells).

### Samples

The methods of the present invention can be used to capture nucleic acid from a wide variety of samples, particularly biological samples, such as body fluids (e.g., whole blood, blood serum, urine, saliva, cerebral spinal fluid, semen, or synovial lymphatic fluid), various tissues (e.g., skin, hair, fur, feces, tumors, or organs such as liver or spleen), cell cultures or cell culture supernatants, etc. The sample can be a food sample, a beverage sample, a fermentation broth, a clinical sample used to diagnose, treat, monitor, or cure a disease or disorder, a forensic sample, an agricultural sample (e.g., from a plant or animal), or an environmental sample (e.g., soil, dirt, or garbage).

Biological samples are those of biological or biochemical origin. Those suitable for use in the methods of the present invention can be derived from mammalian, plant, bacterial, or yeast sources. The biological sample can be in the form of single cells or in the form of a tissue. Cells or tissue can be derived from in vitro culture.

For certain of these embodiments, the sample material includes a plurality of cells. Cells can be prokaryotic or eukaryotic cells, and can include mammalian and non-mammalian animal cells, plant cells, algae, including blue-green algae, fungi, bacteria, protozoa, yeast, and the like. For certain of these embodiments, the cells are bacterial cells.

For certain embodiments, a sample can be subjected to centrifugation, particularly ultracentrifugation, to concentrate the sample prior to subjecting it to a process of the present invention. For example, a sample such as whole blood can be preprocessed by centrifuging and the white blood cells (i.e., the buffy coat) separated from the blood and used as the sample in the methods of the invention.

The sample can be a solid sample (e.g., solid tissue) that is dissolved or dispersed in water or an organic medium, or from which the nucleic acid has been extracted into water or an organic medium. For example, the sample can be an organ homogenate (e.g., liver, spleen). Thus, the sample can include previously extracted nucleic acid (particularly if it is a solid sample).

In the context of the present invention, nucleic acid-containing material refers to a plurality of cells (e.g., white blood cells, bacterial cells, spores), nuclei, viruses, fungi, or any other composition that houses a structure that includes nucleic acid (e.g., plasmid, cosmid, or viroid, archeobacteriae). In certain preferred embodiments of such methods, the nucleic acid-containing material includes nuclei. In certain embodiments, such nuclei are in tact (i.e., substantially unlysed) when they contact the solid phase material described herein.

In certain embodiments, the sample material can include a surfactant. A surfactant can be included for de-clumping cells, improving mixing, enhancing fluid flow, for example, in a device, such as a microfluidic device. The surfactant can be nonionic, such as a poly(ethylene oxide)-polypropylene oxide) copolymer available, for example, under the trade name PLURONIC, polyethylene glycol (PEG), polyoxyethylenesorbitan monolaurate available under the trade name TWEEN 20, 4-(1,1,3,3-tetramethylbutyl)phenyl-polyethylene glycol available under the trade name Triton X-100 anionic, such as lithium lauryl sulfate, N-lauroylsarcosine sodium salt, and sodium dodecyl sulfate; cationic, such as alkyl pyridinium and quaternary ammonium salts; zwitterionic, such as N—(C₁₀-C₁₆ alkyl)-N,N-dimethylglycine betaine (in the betaine family of surfactants); and/or a fluoro surfactant such as FLUORAD-FS 300 (3M, St. Paul, Minn.) and ZONYL (Dupont de Nemours Co., Wilmington, Del.).

In certain embodiments, the sample material can include an enrichment broth to enrich the sample with a microorganism of interest. Typically, this is done for the detection of an antibiotic-resistant microorganism. The sensitivity of a sample for such a microorganism can be enhance by including an enrichment culture process prior to sample preparation to extract the intracellular contents for amplification and detection. Sample material (e.g., a clinical sample) is used to inoculate a suitable medium/broth supplemented with the antibiotic(s) at a certain concentration which kills other microorganisms in the sample but allows for proliferation of the antibiotic-resistant microorganism, and then the culture is incubated at a suitable temperature (e.g., 37° C.) for a period of time (preferably, less than 4 hours for quick enrichment, but this can be overnight for certain slow-growing microorganisms). At the end of the enrichment culture process, the sample with the microorganism of interest is collected from a portion of the culture by centrifugation, filtration or other suitable methods, and then used in methods of the present invention involving amplification and detection.

The nucleic acid can be amplified and used for a wide variety of applications. It can be used for the diagnosis of the presence of a microorganism (e.g., bacteria, virus) in a sample, and subsequently can be used for monitoring and/or remedying the damage caused by the microorganism to the source of the sample. The methods, devices, and kits of the present invention are especially well-suited for analyzing bacteria, including gram positive bacteria, gram negative bacteria, or combinations thereof.

The methods, devices, and kits of the present invention are especially well-suited for preparing nucleic acid extracts for use in amplification techniques used in high throughput or automated processes, particularly microfluidic systems. Such nucleic acid amplification reactions are well known to those skilled in the art and include, for example, Strand Displacement Amplification (SDA), Polymerase Chain Reaction (PCR), Ligase Chain Reaction (LCR), Transcription Mediated Amplification (TMA), Nucleic Acid Sequence Based Amplification (NASBA), as well as bDNA, and INVADER techniques. One or more of such techniques can be used in series in the methods of the present invention.

The nucleic acids may be from an impure, partially pure, or a pure sample. The purity of the original sample is not critical, as nucleic acid may be isolated from even grossly impure samples. For example, nucleic acid may be obtained from an impure sample of a biological fluid such as blood, saliva, or tissue. If an original sample of higher purity is desired, the sample may be treated according to any conventional means known to those of skill in the art prior to undergoing the methods of the present invention. For example, the sample may be processed so as to remove certain impurities such as insoluble materials prior to subjecting the sample to a method of the present invention.

Complex matrices (feces, blood, food matrix, tissue, sputum, etc) may contain solid debris, PCR inhibitors, and/or other nucleic-acid containing species. Solid debris is commonly removed by sedimentation or centrifugation (separate supernatant from solids), filtration, etc. PCR inhibitors are often removed by treatment with protein denaturant or proteases, binding to solid supports, dilution, etc. Undesired nucleic-acid containing species may be reduced by selective lysis, differential centrifugation, filtration, etc. Such sample clean-up may occur on a device described herein or, more typically, prior to delivering the sample material to the device, particularly if the sample includes solid debris. Filtration can occur outside of the device, for example, using a syringe filter or filter plate. Filtration can occur on the device, for example, using centrifugal force to move the sample through a filter in a microfluidic channel.

The nucleic acid captured and amplified as described herein may be of any molecular weight and in single-stranded form, double-stranded form, circular, plasmid, etc. Various types of nucleic acid can be separated from each other (e.g., RNA from DNA, or double-stranded DNA from single-stranded DNA). For example, small oligonucleotides or nucleic acid molecules of about 10 to about 50 bases in length, much longer molecules of about 1000 bases to about 10,000 bases in length, and even high molecular weight nucleic acids of about 50 kb to about 500 kb can be isolated using the methods of the present invention. In some aspects, a nucleic acid isolated according to the invention may preferably be in the range of about 10 bases to about 100 kilobases.

The nucleic acid-containing sample may be in a wide variety of volumes. For example, the applied volume may be as large as 1 liter or as small as 1 μL, or even less. The sample size typically varies depending on the equipment used to carry out the method. For a microfluidic format, typically very small volumes, e.g., 10 μL, (and preferably, no greater than 100 μL) are preferred. It should be understood that larger samples can be used if preprocessed, such as by concentrating.

In the methods of the present invention, a compaction step (e.g., a centrifugation step) to concentrate nucleic acid-containing material and/or nucleic acid is useful for low copy number samples. For low copy number genes, a sample size of up to 100 μL, may be needed. For high copy number genes, a sample size as small as 2 μL can be used, but reproducibility is better with larger volumes (e.g., 20 μL). In the case of smaller volumes, higher throughputs (i.e., number of samples processed per microfluidic device) can be obtained. In the case of larger volumes (e.g., 20 μL), it may not be necessary to go through a pre-spin step for concentration of nucleic acid-containing cells.

The desired nucleic acid captured and amplified using the methods of the present invention is preferably in an amount of at least 20%, more preferably in an amount of at least 30%, more preferably at least 70%, and most preferably at least 90%, of the amount of total nucleic acid in the originally applied sample. Thus, certain preferred methods of the present invention provide for high recovery of the desired nucleic acid from a sample. Furthermore, exceedingly small amounts of nucleic acid may be quantitatively recovered according to the invention.

### Functionalized Support Material

The present invention involves the use of one or more functionalized support materials (i.e., functionalized solid phase materials), preferably nonspecific functionalized support materials, in a device that includes a process array that includes process chambers defining volumes for containing sample material or portions thereof (i.e., nucleic acid-containing material, nucleic acid) and a conduit connecting the process chambers.

In the methods of the present invention, functionalized support material, preferably nonspecific functionalized support material, is used to capture nucleic acid released from nucleic acid-containing material upon lysis. In certain embodiments, functionalized support material, preferably nonspecific functionalized support material, can be used to capture nucleic acid-containing material (e.g., bacterial cells) before or after delivering sample material to the device for lysis. If both the nucleic acid and nucleic acid-containing material are captured in a method of the present invention, the same or different (preferably, nonspecific) functionalized support material can be used (this can be the same or different type of material or the same or different material per se). Preferably, the functionalized support material (preferably, nonspecific functionalized support material) that is used to capture the nucleic acid is different than the functionalized support material (preferably, nonspecific functionalized support material) used to capture the nucleic acid-containing material (e.g., bacteria). Different functionalized support material is desired if lysis is inefficient and/or steric hindrance prevents binding of released nucleic acid on to the same functionalized support material.

Suitable functionalized support materials typically include a solid matrix in any form (e.g., particles, beads, microbeads, microspheres, fibrils) that can be introduced into, and transported through, a microfluidic device (e.g., a device with a process array that includes process chambers defining volumes for containing sample material or portions thereof (i.e., nucleic acid-containing material, nucleic acid) and a conduit connecting the process chambers). Such materials can be magnetic or non-magnetic.

Suitable nonspecific functionalized support materials include capture sites (e.g., functional groups) attached thereto for attachment to nucleic acid-containing material and/or nucleic acid. Such nonspecific functionalizes support materials are not designed to selectively capture inhibitors relative to nucleic acid and/or nucleic acid-containing material. For example, beads can be functionalized with the appropriate groups to capture cells, viruses, bacteria, proteins, nucleic acids, etc. For certain embodiments, the nonspecific functionalized support material can be functionalized to capture bacteria, fungi, or both. For certain embodiments, the nonspecific functionalized support material can be functionalized to capture gram negative bacteria, gram positive bacteria, or both.

Suitable nonspecific functionalized support material does not capture specifically any one particular nucleic acid-containing material or one any particular nucleic acid relative to all others. Although certain nonspecific functionalized support material may capture bacteria as opposed to fungi, for example, there is no specific capture of any one species of bacteria over any other species. This is because the interaction generally results from various mechanisms, such as the attraction between a positively charged bead surface and a negative charged cell surface. There are no functional groups or molecules associated with the nonspecific functionalized support material that are specific for a particular species of microorganism, or a particular nucleic acid, etc.

While the methods of the invention described herein are preferably characterized for nonspecific capture of nucleic acid-containing materials and/or nucleic acids, which is preferred, certain embodiments of the methods may also be used for specific capture of target nucleic acid-containing materials and/or nucleic acids. For example, materials useful for specific capture can include nucleic acid probes having sequences complementary to the target nucleic acids. An example of commercially available beads suitable for capture of bacteria such as Staphylococcus, Streptococcus, E coli, Salmonella, and Clamydia elementary bodies are those sold under the BUGS N BEADS trade name by GenPoint, Oslo, Norway. Antibodies may also be used to specifically capture nucleic acid-containing materials. For example, viral particles can be captured onto beads by covalently attaching antibodies onto bead surfaces. The antibodies can be raised against the viral coat proteins. Commercially available DYNAL beads can be used to covalently link antibodies. Alternatively, synthetic polymers, for example, anion-exchange polymers, can be used to concentrate viral particles. Commercially available resins such as viraffinity (Biotech Support Group, East Brunswick, N.J.) can be used to coat beads or applied as polymer coatings onto select locations in microfluidic device to concentrate viral particles.

The functionalized support material, particularly beads, microbeads, microspheres, or particles, can be segregated from the sample by compaction through, e.g., centrifugation, and subsequent separation. Beads are particularly desirable because they can be designed to have the appropriate density and sizes (nanometers to microns) for segregation. If the particles are magnetic, they can be compacted or otherwise segregated, for example from a detection area of a device, using magnetic forces.

Generally, suitable materials are insoluble in an aqueous environment, chemically inert, physically and chemically stable, and compatible with a variety of biological samples. Examples of solid phase materials include silica, zirconia, alumina beads, metal colloids such as gold, gold coated sheets that have been functionalized through mercapto chemistry, for example, to generate capture sites. Examples of suitable polymers include for example, polyolefin, polystyrene, nylon, poly(meth)acrylate, polyacrylamide, polysaccharide, and fluorinated polymers, as well as resins such as agarose, latex, cellulose, and dextran. The solid phase material is typically washed to remove salts and other contaminants prior to use. It can either be stored dry or in aqueous suspension ready for use.

Preferably, the functionalized support material includes particles, which are preferably uniform, spherical, and rigid to ensure good fluid flow characteristics. The materials are preferably in the form of a loose, porous network to allow uniform and unimpaired entry and exit of large molecules and to provide a large surface area. The particles can be microparticles, which include microspheres, microbeads, and the like. Such particles can be resin particles, for example, agarose, latex, polystyrene, nylon, polyacylamide, cellulose, polysaccharide, or a combination thereof, or inorganic particles, for example, silica, aluminum oxide, or a combination thereof. Such particles can be magnetic or non-magnetic. Such particles can be colloidal in size, for example about 100 nm to about 10 μm. Preferably, for such applications, the solid phase material has a relatively high surface area, such as, for example, more than one meter squared per gram (m²/g).

Examples of commercially available beads include, but are not limited to, crosslinked polystyrene beads available under the trade designation CHELEX from Bio-Rad Laboratories, Inc. (Hercules, Calif.), crosslinked agarose beads with tris(2-aminoethyl)amine, iminodiacetic acid, nitrilotriacetic acid, polyamines and polyimines as well as the chelating ion exchange resins commercially available under the trade designation DUOLITE C-467 and DUOLITE GT73 from Rohm and Haas (Philadelphia, Pa.), AMBERLITE IRC-748, DIAION CR11, DUOLITE C647. Other examples of beads include those available under the trade designations GENE FIZZ (Eurobio, France), GENE RELEASER (Bioventures Inc., Murfreesboro, Tenn.), and BUGS N BEADS (GenPoint, Oslo, Norway), as well as Zymo's beads (Zymo Research, Orange, Calif.) and DYNAL beads (Dynal, Oslo, Norway). Other examples of nonspecific capture materials for nucleic acids and nucleic acid-containing materials are as follows:

| Company Name | Product Name | Publication |
|---|---|---|
| Invitrogen | CHARGESWITCH Genomic DNA | DE 60107468T |
| | Kits | |
| Invitrogen | DYNABEADS DNA DIRECT | U.S. Pat. No. |
| | Universal | 6,617,105 |
| Gen Probe | N/A | WO |
| | | 2008/016988 |
| GenScript | BACREADY DNA Preparation | |
| Corperation | | |
| Norgen Biotech | Urine Bacteria Genomic DNA | U.S. Pat. No. |
| | Isolation Kit | 6,177,278 |
| | | (silica based) |
| AXXORA | SPINCLEAN Genomic DNA | |
| | purification kit | |
| Sigma-Aldrich | GENELUTE Bacterial Genomic | |
| | DNA Miniprep Kit | |
| Roche Applied | MagNA Pure LC DNA Isolation Kit | WO 01/37291 |
| Science | | |
| Qiagen | QIAmp | |
| Epicentre | MASTERPURE Gram Positive DNA | |
| Biotechnology | Purification Kit | |
| Promega | WIZARD Genomic DNA | U.S. Pat. No. |
| | Purification Kit | 6,027,945 |
| MetaBion | mi-Bacterial Genomic DNA Kit | |
| CHEMERx | GeneMATRIX Bacterial Genomic | |
| | Gram +/− DNA Purification Kit | |
| Idaho Technology | IT 1-2-3 SCOOP Sample Purification | |
| Inc. | Kit | |
| GenPoint | Genpoint Live Bacteria/Bacterial | U.S. Pat. No. |
| | DNA Isolation by Magnetic Beads | 6,617,105 |
| Beckman Coulter | AGENCOURT GENFIND Kits | |
| MP Biomedicals | FASTDNA Kit | |

In preferred embodiments, the nonspecific functionalized support material includes an immobilized-metal support material that includes a substrate (i.e., solid phase material, preferably, particulate material) and a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; wherein M is selected from the group consisting of zirconium, gallium, iron, aluminum, scandium, titanium, vanadium, yttrium, and a lanthanide; y is an integer from 3 to 6; and x is 1 or 2. In certain preferred embodiments, the plurality of —C(O)O⁻ or —P(O)(—OH)₂₋ₓ(—O⁻)ₓ groups is a plurality of —C(O)O⁻ groups.

The plurality of —C(O)O⁻ or —P(O)(—OH)₂₋ₓ(—O⁻)ₓ groups can be bound to the substrate in a number of ways. For example, the groups can be bound by covalent bonding, ionic bonding, hydrogen bonding, and/or van der Waals forces. The groups can be bound directly to the substrate, such as a substrate having a polymeric surface wherein a polymer has —C(O)O⁻ or —P(O)(—OH)₂₋ₓ(—O⁻)ₓ groups covalently bonded to the polymer chain. Polymers of this nature can include —C(O)OH or —P(O)(—OH)₂ substituted vinyl units, for example, acrylic acid, methacrylic acid, vinylphosphonic acid, and like units.

The —C(O)O⁻ or —P(O)(—OH)₂₋ₓ(—O⁻)ₓ groups can be bound indirectly to the substrate through a connecting group. For example, amino groups on a substrate can be contacted with a compound having multiple carboxy groups, such as nitrilotriacetic acid, to form an amide-containing connecting group which attaches one or more carboxy groups (two carboxy groups in the case of nitrilotriacetic acid) to the substrate. Substrates having available amino groups or which can be modified to have available amino groups are known to those skilled in the art and include, for example, agarose-based, latex-based, polystyrene-based, and silica-based substrates. Silica-based substrates such as glass or silica particles having —Si—OH groups can be treated with known aminosilane coupling agents, such as 3-aminopropyltrimethoxysilane, to provide available amino groups. Functional groups such as —C(O)OH or —P(O)(—OH)₂ can be attached to a substrate, for example, a substrate having a silica surface, using other known silane compounds.

The —C(O)O⁻ or —P(O)(—OH)₂₋ₓ(—O⁻)ₓ groups can also be bound indirectly to the substrate under conditions where these groups are attached to a molecule which binds to the substrate by electrostatic, hydrogen bonding, coordination bonding, van der Waals forces (hydrophobic interaction) or specific chemistry such as biotin-avdine interaction. For example, polymers bearing C(O)O⁻ or —P(O)(—OH)₂₋ₓ(—O⁻)ₓ groups can be coated on a surface with opposite charge using a Layer-by-Layer technique to build up a high density of polymer having C(O)O⁻ or —P(O)(—OH)₂₋ₓ(—O⁻)ₓ groups.

For a further example, monomers bearing C(O)O⁻ or —P(O)(—OH)₂₋ₓ(—O⁻)ₓ groups can be grafted to a polymer surface through plasma treatment.

Substrates having a plurality of carboxyl groups, e.g., —C(O)OH or —C(O)O⁻, are known and commercially available. For example, carboxylated microparticles are available under trade names such as DYNABEADS MYONE (Invitrogen, Carlsbad, Calif.) and SERA-MAG (Thermo Scientific, known as Seradyn, Indianapolis, Ind.).

The metal ions, M^{y+}, can be bound to acid groups by contacting the acid groups with an excess of metal ions, for example, as a solution of the metal salt, such as a nitrate salt. Other salts may be used as well, for example, chloride, perchlorate, sulfate, phosphate, acetate, acetylacetonate, bromide, fluoride, or iodide, salts.

Such preferred nonspecific functionalized support materials are preferably used in pH range of 4.5 to 6.5, and more preferably a pH range of 5 to 6. The use of the pH range 4.5 to 6.5 may provide increased versatility in the choice of the metal ion, for example, when biological material to the immobilized-metal support material. For example, the metal ion, Ga³⁺ effectively binds bacterial cells at a pH of 4.5 to 6.5, but may release cells at a pH of 7 to 9. A pH in the range of 4.5 to 6.5 can be conveniently provided using a 0.1 M 4-morpholineethanesulfonic acid (MES) buffer at a pH of about 5.5.

In order to minimize interference with methods in which the compositions of the present invention may be used, appreciable levels of a salt may not be included. Appreciable level(s) refers to a level greater than about 0.2 M, and more preferably a level greater than about 0.1 M. For certain embodiments, when a salt is present in the composition at an appreciable level, any salt included at an appreciable level in the composition is other than an inorganic salt or a one to four carbon atom-containing salt.

The metal ion, M^{y+}, is chosen so that the metal ion can bind the phosphate portion of a nucleic acid (i.e., polynucleotide) sufficiently to bind the nucleic acid present in a sample material. Moreover, the metal ion is also chosen to allow competitive binding with a metal-chelating reagent in a wash buffer to efficiently, preferably quantitatively, release or elute the nucleic acid from the immobilized-metal support material at a low reagent concentration and under mild conditions. A low reagent concentration without the addition of any salt to increase the ionic strength can be about 0.1 M or less, 0.05 M or less, or 0.025 M or less. Mild conditions can include the low reagent concentration, a pH of about 7 to 10, a temperature of not more than about 95° C., preferably not more than about 65° C., or a combination thereof.

For certain embodiments, M is selected from the group consisting of zirconium, gallium, iron, aluminum, scandium, titanium, vanadium, yttrium, and a lanthanide. A lanthanide includes any one of the lanthanide metals: lanthanum, cerium, praseodymium, promethium, samarium, europium, gadolinium, terbium, dysprosium, holmium, erbium, thulium, ytterbium, and lutetium. Lanthanum and cerium are preferred lanthanides. For certain of these embodiments, M is selected from the group consisting of zirconium, gallium, iron, aluminum, scandium, titanium, vanadium, lanthanum, and cerium. For certain of these embodiments, M is selected from the group consisting of zirconium, gallium, and iron. For certain of these embodiments, M is zirconium.

For certain embodiments, y is 3 or 4. For certain embodiments, M^{y+} is Zr⁴⁺ or Ga³⁺. For certain of these embodiments, M^{y+} is Zr⁴⁺. Such functionalized support material is described in greater detail in U.S. Patent Application Ser. No. 60/913,812 (Attorney Docket No. 62809US002), filed on Apr. 25, 2007, and Ser. No. ______ (Attorney Docket No. 62809WO003), filed on even date herewith, and entitled “COMPOSITIONS, METHODS, AND DEVICES FOR ISOLATING BIOLOGICAL MATERIALS.”

### Lysing Reagents and Conditions

For certain embodiments of the invention, at some point during the process, nucleic acid-containing material (particularly cells such as bacterial cells) are lysed to release nucleic acid. Lysis herein is the physical disruption of the membranes of the cells, referring to the outer cell membrane and, when present, the nuclear membrane. This can be done using standard techniques and reagents, e.g., an enzyme and/or base (preferably, a strong base).

Typically, a lysing reagent (i.e., lysing agent) is in aqueous media, such as a lysis buffer, which can provide a pH of at least 4.5 (preferably at least 7). Herein, preferably lysis is carried out using a lysis buffer that includes at least one lysing reagent, and in certain embodiments at least two lysing reagents (e.g., a lysing reagent for gram negative bacteria and a lysing reagent for gram positive bacteria). The lysing reagents can include, for example, an enzyme, a base (i.e., alkali or alkali lysing reagent), or a surfactant (i.e., a detergent). Various combinations of lysing reagents can be used if desired. For example, in certain embodiments, a combination of an enzyme and a surfactant can be used, or a combination of a base and a surfactant (e.g., an ionic surfactant) can be used. Heating can be used in certain embodiments to enhance lysing (e.g., heating with Proteinase K).

Enzymatic lysis is a particularly desirable lysis method. Enzymes used for lysis include, for example, lysostaphin, lysozyme, mutanolysin, proteinases (e.g., Proteinase K), pronases, cellulases, cell wall peptidoglycan degrading enzyme, or combinations thereof.

Lysing enzymes can be used for selective lysis if desired. For example, a cell wall degrading enzyme can lyse different microorganisms with different efficiencies, depending on the concentration of the enzyme and/or the time allowed for lysis, since the cell wall structural proteins or cross-linkages vary in different microorganisms. That is, the concentration of the cell wall degrading enzyme and the time of the lysis process determine whether a certain microorganism can be lysed. For example, lysostaphin at a certain concentration lyses Staphylococcus aureus but not Staphylococcus epidermidis in 5 minutes. The selectively lysed microorganisms release their intracellular contents which are captured and then used as targets for amplification and detection; however, the un-lysed microorganisms are removed during the washings of targets captured on a solid support material.

Chemical lysis can be carried out using a base (i.e., alkali or alkaline reagent). In such alkaline lysis methods, a neutralization reagent may be used to neutralize the solution or mixture after lysis. Typically, a strong base can be used to lyse any nuclei contained in nucleic acid-containing cells (as in white blood cells) to release nucleic acid. A strong base refers to a base that is completely dissociated in water. Generally, a wide variety of strong bases can be used to create an effective pH (e.g., 8-13, preferably 13) in an alkaline lysis procedure. The strong base is typically a hydroxide such as NaOH, LiOH, KOH, or a hydroxide with a quaternary nitrogen-containing cation (e.g., quaternary ammonium) such as NH₄OH, or a base such as a primary, secondary, or tertiary amine, or combinations thereof. Typically, the concentration of the strong base is at least 0.01 Normal (N), and typically, no more than 1 N.

With alkaline lysis, typically, the mixture is neutralized prior to amplification. Suitable neutralization reagents include, for example, an inorganic acid solution or an acidic buffer. Specific examples include TRIS-HCl (pH 7.5), MES (pH 5.5), PIPS, succinate acid, citrate buffers, and the like. Suitable neutralization reagents are used subsequent to lysis. Thus, in methods of the present invention wherein the lysing reagent (typically included in a lysis buffer) is a basic lysing reagent, the methods further include contacting the released nucleic acid, optionally attached to functionalized support material (preferably, nonspecific functionalized support material), with a neutralization reagent. Typically, the amount of nucleic acid that is captured by the functionalized support material (preferably, nonspecific functionalized support material) can be increased using a neutralization reagent.

A surfactant can be used to lyse nucleic acid-containing material, particularly cells (e.g., white blood cells (WBC's), bacterial cells, viral cells), to release nucleic acid. Nonionic surfactants, cationic surfactants, anionic surfactants, and zwitterionic surfactants can be used to lyse cells. Particularly useful are nonionic surfactants. The surfactant can be nonionic, such as a poly(ethylene oxide)-polypropylene oxide) copolymer available, for example, under the trade name PLURONIC, polyethylene glycol (PEG), polyoxyethylenesorbitan monolaurate available under the trade name TWEEN 20, 4-(1,1,3,3-tetramethylbutyl)phenyl-polyethylene glycol available under the trade name Triton X-100; anionic, such as lithium lauryl sulfate (LLS), N-lauroylsarcosine sodium salt, and sodium dodecyl sulfate (SDS); cationic, such as alkyl pyridinium and quaternary ammonium salts; zwitterionic, such as N—(C₁₀-C₁₆alkyl)-N,N-dimethylglycine betaine (in the betaine family of surfactants); and/or a fluoro surfactant such as FLUORAD-FS 300 (3M, St. Paul, Minn.) and ZONYL (Dupont de Nemours Co., Wilmington, Del.). Other suitable surfactants include those of the TRITON series, TWEEN series, BRIJ series, NP series, CHAPS, N-methyl-N-(1-oxododecyl)glycine, sodium salt, or the like. Combinations of surfactants can be used if desired. For example, a nonionic surfactant such as TRITON X-100 can be added to a TRIS buffer containing sucrose and magnesium salts.

The amount of surfactant used for lysing is sufficiently high to effectively lyse the sample, yet sufficiently low to avoid precipitation, for example. The concentration of surfactant used in lysing procedures is typically at least 0.1 wt-%, based on the total weight of the sample. The concentration of surfactant used in lysing procedures is typically no greater than 4.0 wt-%, and preferably, no greater than 1.0 wt-%, based on the total weight of the sample. The concentration is preferably optimized in order to obtain complete lysis in the shortest possible time with the resulting mixture being PCR compatible.

Other components in a lysis buffer, in addition to one or more lysing reagents, can include a chaotrope such as guanidinium hydrochloride, guanidine isothiocyanate, urea, sodium iodide, or the like; a chelator such as ethylenediaminetetraacetic acid (EDTA), ethylene glycol-bis(2-aminoethylether)-N,N,N′N′-tetraacetic acid (EGTA), other suitable metal ion chelators, or the like; an RNase or DNase inhibitor such as RNasin (RNA inhibitor from Promega) or aurintricarboxylic acid (DNase inhibitor); a protein denaturant such as guanidinium hydrochloride, guanidine isothiocyanate, urea, or the like. Various combinations of such additional lysis buffer components can be used if desired. Such reagents can be used in the methods of the present invention separate from the lysis buffer, if desired.

Lysing conditions depend on the lysing reagent (typically in a lysis buffer). For example, if the lysing reagent includes a strong base, the conditions include subjecting the nucleic acid-containing material to a strong base and subsequent neutralization reagent with optional heating. Typically, the temperature is at room temperature. If a base is used, the sample containing the released nucleic acid may need to have its pH adjusted, particularly if the nucleic acid is to be subjected to a subsequent amplification process. Thus, certain embodiments of the invention include adjusting the pH of the sample typically to at least 7.5, and typically to no greater than 9.

In one preferred embodiment, the lysis buffer includes an enzyme (e.g., proteinase K or other suitable proteases) and a surfactant (e.g., sodium dodecyl sulfate (SDS) or other nonionic detergents). For example, a lysis buffer for bacteria can include proteinase K or other suitable protease lysing reagent, and sodium dodecyl sulfate (SDS) or other detergents (preferably nonionic detergents).

In one embodiment, the lysis buffer includes a protease lysing reagent, a surfactant (preferably a nonionic detergent), and optionally a chelator. For example, the lysis buffer for mammalian tissue or cells or a virus can include proteinase K or other suitable protease lysing reagent, sodium dodecyl sulfate (SDS) or other detergents (preferably nonionic detergents), and a chelator (e.g., ethylenediaminetetraacetic acid (EDTA) or other suitable metal ion chelators).

In one embodiment, the lysis buffer includes a protease lysing reagent, a detergent (preferably a nonionic detergent), a chelator, and optionally a protein denaturant. For example, a universal cell lysis buffer includes guanidine hydrochloride or guanidine isothiocyanate or other suitable protein denaturant, proteinase K or other suitable protease that retains enzymatic activities in the presence of the protein denaturant, EDTA or other suitable metal ion chelator, and nonionic detergents such as PLURONIC L-64, TWEEN 20, or TRITON X-100.

In one embodiment, the lysis buffer includes a cell wall peptidoglycan degrading enzyme, a surfactant (preferably non-ionic detergent), a chelator, and an RNase or DNase inhibitor. For example, for bacterial cell lysis, the lysis buffer includes a cell wall peptidoglycan degrading enzyme such as lysostaphin, lysozyme, or other suitable enzymes, EDTA or other suitable metal ion chelators, PLURONIC L-64 (from BASF) or other nonionic detergents, and RNase inhibitors (for RNA isolation) such as RNasin (from Promega) or DNase inhibitors (for DNA isolation) such as aurintricarboxylic acid.

In one embodiment, the lysis buffer includes guanidine isothiocyanate. For example, a mono-phasic cell lysis buffer contains phenol and guanidine isothiocyanate (e.g., TRIzol Reagent (Invitrogen)), followed by chloroform extraction and centrifugation to separate the organic and aqueous phases.

One can also use a commercially available lysing reagent and neutralization agent such as in Sigma's Extract-N-Amp Blood PCR kit scaled down to microfluidic dimensions. Stonger lysing solutions such as POWERLYSE from GenPoint (Oslo, Norway) for lysing difficult bacteria such as Staphylococcus, Streptococcus, etc., can be used to advantage in certain methods of the present invention.

For certain embodiments, a higher chain alcohol can be used for precipitating nucleic acid in the presence of functionalized support material. For example, polyethylene glycol can be used. Typically different molecular weights (e.g., from 2,000 to 1,000,000) as well as concentrations (e.g., 2% to 50%) of such alcohols can be used for precipitation of the nucleic acid in the presence of the functionalized support material. For example, a nucleic acid-containing sample such as blood can be mixed with dried lysis reagent containing a proteinase K, optionally a guanidinium salt, polyethylene glycol, and silica beads. The nucleic acid-containing sample is then mixed (and optionally heated) for lysis to occur. In some cases, a certain volume of buffer (up to 30 μL, for example) can be introduced from a buffer chamber to dilute the sample prior to lysis. The sample binds to the beads, and washing is carried out with approximately 5 μL of bead suspension moved into the next chamber for further processing.

Bacterial cell lysis, depending on the type, may be accomplished using enzymatic methods (e.g., lysozyme, lysostaphin, mutanolysin) or chemical methods such as alkaline lysis. In a specific example of a protocol for bacterial cultures, which can be incorporated into a microfluidic device, an E. Coli cell culture is centrifuged and resuspended in TE buffer (10 mM TRIS, 1 mM EDTA, pH 7.5) and lysed by the addition of 0.1 M NaOH/1% SDS (sodium dodecyl sulfate). The cell lysis is stopped by the addition of 1 volume of 3 M (three molar) potassium acetate (pH 4.8) and the supernatant centrifuged. The cell lysate is further purified to get clean plasmid DNA.

Plasma and serum represent the majority of specimens submitted for molecular testing that include viruses. After fractionation of whole blood, plasma, or serum samples can be used for the extraction of viruses (i.e., viral particles). For example, to isolate DNA from viruses, it is possible in the microfluidic case, to first separate out the serum by spinning blood. By the use of the variable valve, which is described in greater detail below, the serum alone can be emptied into another chamber. The serum can then be centrifuged to concentrate the virus or can be used directly in subsequent lysis. The virus can be lysed by enzymatic or chemical means, for example, by the use of surfactants. In cases where viral RNA is required, it may be necessary to have an RNAse inhibitor added to the solution to prevent degradation of RNA.

### Illustrative Methods Including Amplification

In one embodiment, the present invention provides a method of amplifying nucleic acid, wherein the method includes: providing a device that includes a process array having process chambers defining volumes for containing sample material or portions thereof (i.e., nucleic acid-containing material, nucleic acid) and a conduit connecting the process chambers; delivering sample material including nucleic acid-containing material (e.g., bacteria) to a process chamber of the device; optionally contacting the nucleic acid-containing material with functionalized support material (preferably, nonspecific functionalized support material) under conditions effective to capture at least a portion of the nucleic acid-containing material by the functionalized support material in the process chamber; optionally transferring the functionalized support material and captured nucleic acid-containing material to a different process chamber; contacting the nucleic acid-containing material, optionally attached to the functionalized support material, with at least one lysing reagent (that can be in a lysis buffer) under conditions (e.g., time, temperature, reagents) effective to lyse at least a portion of the nucleic acid-containing material and release nucleic acid; in the same or a different process chamber, contacting the released nucleic acid with functionalized support material (preferably, nonspecific functionalized support material), which may be the same or different than the functionalized support material optionally used to capture the nucleic acid-containing material, under conditions effective to capture at least a portion of the nucleic acid by the same or different functionalized support material; washing the functionalized support material with nucleic acid attached thereto (for example, using multiple aliquots of one or more wash solutions, which can be carried out using a thermopipetting process); transferring the functionalized support material with nucleic acid attached thereto to a different process chamber through the conduit by rotating the device; before, during, or after transferring the functionalized support material with nucleic acid attached thereto, contacting it with an amplification reagent (which may be located in the different process chamber or in a separate chamber), wherein the amplification reagent includes one or more primers and optionally one or more probes (which are preferably present); optionally heating the functionalized support material (nonspecific functionalized support material) with nucleic acid attached thereto at a temperature effective to release and/or denature (preferably, release and denature) at least a portion of the captured nucleic acid in the presence of the amplification reagent; and contacting the nucleic acid, which is optionally released and/or denatured from the functionalized support material, and the amplification reagent with one or more amplification enzymes under conditions effective to amplify (e.g., time, temperature, reagents) the nucleic acid to produce amplicons. In this method, the optional steps of contacting the nucleic acid-containing material with functionalized support material (preferably, nonspecific functionalized support material), and transferring the functionalized support material and captured nucleic acid-containing material, are preferably included in the process.

In another embodiment, the present invention provides a method of amplifying nucleic acid, wherein the method includes: providing sample material including nucleic acid-containing material; contacting the nucleic acid-containing material with functionalized support material (preferably, nonspecific functionalized support material) under conditions effective to capture at least a portion of the nucleic acid-containing material by the functionalized support material; providing a device that includes a process array having process chambers defining volumes for containing sample material or portions thereof (i.e., nucleic acid-containing material, nucleic acid) and a conduit connecting the process chambers; delivering functionalized support material and captured nucleic acid-containing material to a process chamber of the device; contacting the captured nucleic acid-containing material with at least one lysing reagent (that can be in a lysis buffer) in the process chamber under conditions (e.g., time, temperature, reagents) effective to lyse at least a portion of the nucleic acid-containing material and release nucleic acid; in the same or a different process chamber, contacting the released nucleic acid with functionalized support material (preferably, nonspecific functionalized support material), which may be the same or different than the functionalized support material used to capture the nucleic acid-containing material, under conditions effective to capture at least a portion of the nucleic acid by the same or different functionalized support material; washing the functionalized support material with nucleic acid attached thereto (for example, using multiple aliquots of one or more wash solutions, which can be carried out using a thermopipetting process); transferring the functionalized support material with nucleic acid attached thereto to a different process chamber through the conduit by rotating the device; before, during, or after transferring the functionalized support material with nucleic acid attached thereto, contacting it with an amplification reagent (which may be located in the different process chamber or in a separate chamber), wherein the amplification reagent includes one or more primers and optionally one or more probes (which are preferably present); optionally heating the functionalized support material (preferably, nonspecific functionalized support material) with nucleic acid attached thereto at a temperature effective to release and/or denature (preferably, release and denature) at least a portion of the captured nucleic acid in the presence of the amplification reagent; and contacting the nucleic acid, which is optionally released and/or denatured from the functionalized support material, and the amplification reagent with one or more amplification enzymes under conditions effective to amplify (e.g., time, temperature, reagents) the nucleic acid to produce amplicons.

In these methods, the functionalized support material (preferably, nonspecific functionalized support material) with nucleic acid attached thereto is preferably heated at a temperature effective to release and/or denature the nucleic acid in the presence of the amplification reagent, and more preferably, at a temperature effective to release and denature at least a portion of the captured nucleic acid. When the functionalized support material (preferably, nonspecific functionalized support material) is not heated, the first round of amplification occurs with the captured nucleic acid and subsequent rounds of amplification occur on the resultant products formed in solution.

In these methods, prior to contacting the nucleic acid and the amplification reagent with one or more amplification enzymes, the method preferably includes transferring the nucleic acid to a different process chamber containing the one or more amplification enzymes.

In another embodiment, the present invention provides a method of amplifying nucleic acid, wherein the method includes: providing a device that includes a process array having process chambers defining volumes for containing sample material or portions thereof (i.e., nucleic acid-containing material, nucleic acid) and a conduit connecting the process chambers; delivering sample material that includes nucleic acid-containing material (e.g., bacteria) to a process chamber of the device; optionally contacting the nucleic acid-containing material with functionalized support material (preferably, nonspecific functionalized support material) under conditions effective to capture at least a portion of the nucleic acid-containing material by the functionalized support material in the process chamber; optionally transferring the functionalized support material (preferably, nonspecific functionalized support material) and captured nucleic acid-containing material to a different process chamber; contacting the nucleic acid-containing material, optionally attached to the functionalized support material (preferably, nonspecific functionalized support material), with at least one lysing reagent (that can be in a lysis buffer) under conditions (time, temperature, reagents) effective to lyse at least a portion of the nucleic acid-containing material and release nucleic acid; in the same or a different process chamber, contacting the released nucleic acid with functionalized support material (preferably, nonspecific functionalized support material), which may be the same or different than the functionalized support material optionally used to capture the nucleic acid-containing material, under conditions effective to capture at least a portion of the nucleic acid by the same or different functionalized support material; washing the functionalized support material with nucleic acid attached thereto (for example, using multiple aliquots of one or more wash solutions, which can be carried out using a thermopipetting process); transferring the functionalized support material with nucleic acid attached thereto to a different process chamber through the conduit by rotating the device; wherein the nucleic acid contacts an amplification reagent located in the different process chamber; wherein the amplification reagent includes one or more primers, optionally one or more probes (which are preferably present), and one or more amplification enzymes; and wherein the one or more amplification enzymes are thermally stable at the temperature effective to release and/or denature the nucleic acid; heating the functionalized support material with nucleic acid attached thereto at a temperature effective to release and/or denature (preferably, release and denature) the nucleic acid in the presence of the amplification reagent; and providing conditions effective to amplify (e.g., time, temperature, reagents) the released and/or denatured nucleic acid to produce amplicons. In this method, the optional steps of contacting the nucleic acid-containing material with functionalized support material (preferably, nonspecific functionalized support material), and transferring the functionalized support material and captured nucleic acid-containing material, are preferably included in the process.

In another embodiment, the present invention provides a method of amplifying nucleic acid, wherein the method includes: providing sample material that includes nucleic acid-containing material; contacting the nucleic acid-containing material with functionalized support material (preferably, nonspecific functionalized support material) under conditions effective to capture at least a portion of the nucleic acid-containing material by the functionalized support material; providing a device having a process array that includes process chambers defining volumes for containing sample material or portions thereof (i.e., nucleic acid-containing material, nucleic acid) and a conduit connecting the process chambers; delivering functionalized support material and captured nucleic acid-containing material to a process chamber of the device; contacting the nucleic acid-containing material with at least one lysing reagent (that can be in a lysis buffer) in the process chamber under conditions (e.g., time, temperature, reagents) effective to lyse at least a portion of the captured nucleic acid-containing material and release nucleic acid; in the same or a different process chamber, contacting the released nucleic acid with functionalized support material (preferably, nonspecific functionalized support material), which may be the same or different than the functionalized support material used to capture the nucleic acid-containing material, under conditions effective to capture at least a portion of the nucleic acid by the same or different functionalized support material; washing the functionalized support material with nucleic acid attached thereto (for example, using multiple aliquots of one or more wash solutions, which can be carried out using a thermopipetting process); transferring the functionalized support material with nucleic acid attached thereto to a different process chamber through the conduit by rotating the device; wherein the nucleic acid contacts an amplification reagent located in the different process chamber; wherein the amplification reagent includes one or more primers, optionally one or more probes (which are preferably present), and one or more amplification enzymes; and wherein the one or more amplification enzymes are thermally stable at the temperature effective to release and/or denature the nucleic acid; heating the functionalized support material with nucleic acid attached thereto at a temperature effective to release and/or denature (preferably, release and denature) the nucleic acid in the presence of the amplification reagent; and providing conditions effective to amplify the released and/or denatured nucleic acid to produce amplicons.

In certain preferred embodiments, the nucleic acid-containing material includes bacterial cells, and the method includes: delivering sample material that includes bacterial cells to a process chamber; contacting the bacterial cells with functionalized support material (preferably, nonspecific functionalized support material) under conditions effective to capture at least a portion of the bacterial cells by the functionalized support material in the process chamber; transferring the functionalized support material and captured bacterial cells to a different process chamber; and contacting the bacterial cells attached to the functionalized support material, with a lysis buffer under conditions effective to lyse at least a portion of the bacterial cells and release nucleic acid; in a different process chamber, contacting the released nucleic acid with functionalized support material (preferably, nonspecific functionalized support material), which may be the same or different than the functionalized support material optionally used to capture the bacterial cells, under conditions effective to capture at least a portion of the nucleic acid by the same or different functionalized support material. Preferably, the functionalized support material (preferably, nonspecific functionalized support material) used to capture the bacterial cells and the functionalized support material (preferably, nonspecific functionalized support material) used to capture the nucleic acid are different materials.

If desired, each of the methods described herein can involve lysis outside of the device with the remaining steps occurring on the device. If desired, each of the methods that involve lysing outside of the device can also include capture of the nucleic acid by a functionalized support material (preferably, nonspecific functionalized support material) outside of the device with the remaining steps occurring on the device.

In certain methods, the conditions effective to capture at least a portion of the nucleic acid-containing material by the functionalized support material (preferably, nonspecific functionalized support material) include a pH of 4.5 to 9 (preferably a pH of 5 to 7, and in certain embodiments a pH of 5.5 is particularly preferred). A wide variety of suitable buffers can be used to produce the desired pH (e.g., MES (pH 5.5)). Other conditions for effective capture typically include room temperature and adequate mixing.

In certain methods, the conditions effective to capture at least a portion of the nucleic acid by the functionalized support material (preferably, nonspecific functionalized support material) include a pH of 4.5 to 9 (preferably a pH of 5 to 8, and in certain embodiments a pH of 5.5 is particularly preferred). A wide variety of suitable buffers can be used to produce the desired pH (e.g., MES (pH 5.5)). If capture occurs in the same chamber as lysing (and substantially immediately thereafter), then it may be desirable to have a pH toward the higher end of these ranges. Other conditions for effective capture typically include room temperature and adequate mixing.

Preferably, mixing facilitates lysis and/or nucleic acid capture. Subsequent to capture of the nucleic acid, the functionalized support material (preferably, nonspecific functionalized support material) can be compacted. This can occur, for example, by centrifugation (e.g., by alternating speeds during centrifugation). Alternatively, if the functionalized support material (preferably, nonspecific functionalized support material) is magnetic, temporary application of a magnetic field can be used to facilitate the mixing by centrifugation. The lysing reagent (which can be in a lysis buffer) and unbound sample can then be removed from the compacted support material having captured nucleic acid.

In certain embodiments, methods of the present invention include washing the functionalized support material (preferably, nonspecific functionalized support material) with nucleic acid-containing material attached thereto. Such washing includes the use of one or more wash solutions, preferably, one or more aqueous wash buffers (e.g., one having a pH of 4.5 to 9, and preferably a pH of 4.5 to 6.5).

In certain embodiments, methods of the present invention include washing the functionalized support material (preferably, nonspecific functionalized support material) with nucleic acid attached thereto. Such washing includes the use of a wash buffer (e.g., one having a pH of 4.5 to 9, and preferably a pH of 4.5 to 6.5).

Preferably, washing the functionalized support material (preferably, nonspecific functionalized support material) with nucleic acid attached thereto includes: introducing a wash buffer (e.g., one having a pH of 4.5 to 9) into the process chamber and mixing to form a mixture; optionally compacting the functionalized support material (preferably, nonspecific functionalized support material) with nucleic acid-containing material (or nucleic acid) attached thereto after mixing; removing a majority of the components of the mixture leaving the functionalized support material with nucleic acid-containing material (or nucleic acid) attached thereto in the process chamber; and optionally repeating the introducing and removing steps one or more times (e.g., up to five times).

Suitable wash solutions (typically wash buffers) include, for example, MES (4-morpholineethanesulfonic acid) buffer, Tris buffer, HEPES buffer, phosphate buffer, TAPS buffer, and DIPSO (3-(N,N-bis[2-hydroxyethyl]amino)-2-hydroxypropanesulfonic acid) buffer. Isopropanol can be used to wash in certain embodiments,

Preferably, washing further includes compacting the functionalized support material (preferably, nonspecific functionalized support material) with nucleic acid attached thereto after mixing to form a mixture. This can occur, for example, by centrifugation. Alternatively, if the functionalized support material (preferably, nonspecific functionalized support material) is magnetic, this can occur by application of a magnetic field. The wash buffer, lysing reagent (which can be in a lysis buffer), and unbound sample can then be removed from the compacted support material having captured nucleic acid. Such compaction allows for the transfer of the functionalized support material in as small a volume of liquid as possible (e.g., in approximately 2 microliters to 15 microliters).

Such compaction can occur after multiple washing steps and prior to heating the functionalized support material (preferably, nonspecific functionalized support material) with nucleic acid attached thereto. Alternatively or additionally, after heating the functionalized support material (preferably, nonspecific functionalized support material) with nucleic acid attached thereto to release the nucleic acid, the method further includes compacting the functionalized support material, which allows for separation of the functionalized support material from the nucleic acid.

Washing can be carried out in one or more steps (preferably, with multiple aliquots (i.e., two or more aliquots) of wash solution (e.g., wash buffer) with decanting between addition of each aliquots, with the option of decanting to dryness). Typically, a suitable amount of washing is done to remove inhibitors that can interfere with subsequent amplification and/or detection of the nucleic acid. Generally, three aliquots of 100 microliters each are desired with a majority of the wash buffer (typically at least about 90%) removed (e.g., by decanting) each time from the functionalized support material with nucleic acid and/or nucleic acid-containing material attached thereto. In certain embodiments up to 100% of such wash buffer can be removed leaving the functionalized support material substantially dry.

The functionalized support material (preferably, nonspecific functionalized support material) with nucleic acid attached thereto is preferably transferred to a different process chamber for amplification through a channel by rotating the device. This process chamber includes an amplification reagent. Herein, an “amplification reagent” includes one or more of the components necessary for amplification of nucleic acid. For example, for certain embodiments, the amplification reagent includes one or more the amplification primers and optionally one or more amplification probes (preferably, one or more probes are present). Suitable amplification primers and probes are thermally stable (in the dry or wet state) at the temperatures used to denature and/or release the nucleic acid.

Suitable amplification primers typically include 15 to 30 nucleotide units, which determines the region (targeted sequence) of a nucleic acid to be amplified. Under appropriate conditions, the bases in the primer bind to complementary bases in the region of interest, and then the nucleic acid amplification enzyme extends the primer as determined by the targeted sequence. A large number of primers are known and commercially available, and others can be designed and made using known methods.

Suitable amplification probes allow detection of amplification products (amplicons). This can occur by fluorescing, for example, and thereby generating a detectable signal, the intensity of which is dependent upon the number of probe molecules, e.g., fluorescing probe molecules. Probe molecules can include an oligonucleotide and a fluorescing group, for example, coupled with a quenching group. Probes can fluoresce when separation or decoupling of the quenching group and the fluorescing group occurs upon binding to an amplicon or upon nucleic acid amplifying enzyme cleavage of the probe bound to the amplicon. Alternatively, a probe bound to the amplicon can fluoresce upon exposure to light of an appropriate wavelength. For certain embodiments, including any one of the above embodiments, the probe is selected from the group consisting of TAQMAN probes (Applied Biosystems, Foster City, Calif.), molecular beacons, SCORPIONS probes (Eurogentec Ltd., Hampshire, UK), SYBR GREEN (Invitrogen, Carlsbad, Calif.), FRET hybridization probes (Roche Applied Sciences, Indianapolis, Ind.), Quantitect probes (Qiagen, Valencia, Calif.), and molecular torches.

In these methods, the functionalized support material (preferably, nonspecific functionalized support material) with nucleic acid attached thereto is preferably heated at a temperature effective to release and/or denature the nucleic acid in the presence of the amplification reagent, and more preferably, at a temperature effective to release and denature at least a portion of the captured nucleic acid. When the functionalized support material (preferably, nonspecific functionalized support material) is not heated, the first round of amplification occurs with the captured nucleic acid and subsequent rounds of amplification occur on the resultant products formed in solution.

In this process chamber, in the presence of the amplification primers and optional probes, the functionalized support material (preferably, nonspecific functionalized support material) with nucleic acid attached thereto is preferably heated at a temperature effective to release and/or denature at least a portion of the nucleic acid (e.g., at a temperature of 50° C. to 99° C.).

Although the nucleic acid can be released and/or denatured for subsequent amplification, it is preferred that at least a portion of the nucleic acid be released and denatured from the functionalized support material (preferably, nonspecific functionalized support material).

Preferably, in this same process chamber, the denatured and/or released (preferably, denatured and released) nucleic acid and amplification reagent (one or more primers and optionally one or more probes) are contacted with an amplification enzyme (or mixture of amplification enzymes) under conditions effective to amplify the nucleic acid. Typically, this includes adding an amplification enzyme (or mixture of amplification enzymes) to the process chamber. In certain embodiments, contacting the released and/or denatured nucleic acid and amplification reagent (one or more primers and optionally one or more probes) with an amplification enzyme under conditions effective to amplify the nucleic acid includes heating or cooling the denatured nucleic acid and amplification reagent to an appropriate amplification temperature prior to contacting them with the amplification enzyme. Preferably, this includes a temperature of typically around 42° C. for TMA, although other temperatures can be used depending on the type of amplification reaction selected. Such temperatures are well known to those of skill in the art.

A nucleic acid “amplification enzyme” or “amplifying enzyme” refers to an enzyme that can catalyze the production of a nucleic acid from an existing DNA or RNA template. For certain embodiments, the nucleic acid amplification enzyme is an enzyme that can be used in a process for amplifying a nucleic acid or a portion of a nucleic acid. For certain embodiments, the nucleic acid amplification enzyme is selected from the group consisting of a DNA and/or RNA polymerase and a reverse transcriptase. For certain embodiments, the DNA polymerase is selected from the group consisting of Taq DNA polymerase, Tfl DNA polymerase, Tth DNA polymerase, Tli DNA polymerase, and Pfu DNA polymerase. For certain of these embodiments, the reverse transcriptase is selected from the group consisting of AMV reverse transcriptase, M-MLV reverse transcriptase, and RNase H minus. Retroviral reverse transcriptase, such as M-MLV and AMV posses an RNA-directed DNA polymerase activity, a DNA directed polymerase activity strand displacement activity as well as an RNase H activity. For certain embodiments, the nucleic acid amplification enzyme is a DNA polymerase or an RNA polymerase. For certain embodiments, the nucleic acid amplification enzyme is Taq DNA polymerase. For certain embodiments, the nucleic acid amplifying enzyme is T7 RNA polymerase or Thermus Thermostable RNA polymerase.

In addition to amplification primers, probes, and enzymes, amplification reactions typically include the use of nucleotide triphosphates (NTPs), including ribonucleotide triphosphates and deoxyribonucleotides triphosphates, in the production of a nucleic acid from an existing DNA or RNA template. For example, when amplifying a DNA, a dNTP (deoxyribonucleotide triphosphate) set is used, which typically includes dATP (2′-deoxyadenosine 5′-triphosphate), dCTP (2′-deoxycytodine 5′-triphosphate), dGTP (2′-deoxyguanosine 5′-triphosphate), and dTTP (2′-deoxythimidine 5′-triphosphate).

In certain embodiments, the methods of the present invention include transferring the functionalized support material (preferably, nonspecific functionalized support material) with nucleic acid attached thereto to a different process chamber through the conduit by rotating the device. In this process chamber the nucleic acid contacts an amplification reagent that includes one or more primers, optionally one or more probes, and one or more amplification enzymes. In these embodiments, the amplification primers, optional amplification probes, and amplification enzymes are thermally stable (in the dry or wet state) at the temperatures used to denature and/or release the nucleic acid. Examples of suitable primers, probes, and nucleotide triphosphates are listed above.

Suitable thermally stable amplification enzymes are those enzymes that are able to function at temperatures (typically 60° C. and above, often up to and including 99° C.) that would denature enzymes taken from most “normal” organisms. They can be isolated from some thermophilic microbes (thermophiles, hyperthermophiles, or extremophiles) or prepared through over-expression of some cloned genes of the thermophilic microbes. Examples of suitable thermally stable amplification enzymes include Taq polymerase or a hot-start enzyme (one that activates only after exposure to very high temperatures) such as those commercially available from companies such as Roche, Qiagen, Promega, Invitrogen, and Stratagene.

In these embodiments, the methods include heating the functionalized support material (preferably, nonspecific functionalized support material) with nucleic acid attached thereto at a temperature effective to release and/or denature (preferably, release and denature) at least a portion of the nucleic acid (e.g., at a temperature of 50° C. to 99° C.) in the presence of the amplification reagent (including the one or more thermally stable amplification enzymes); and subsequently providing conditions effective to amplify the released and/or denatured nucleic acid. Such conditions effective to amplify the nucleic acid include heating or cooling the denatured nucleic acid and amplification reagent (including one or more thermally stable amplification enzymes) to an appropriate amplification temperature. Preferably, this includes a temperature chosen for the type of amplification reaction selected. Such temperatures are well known to those of skill in the art.

Buffers can be used to regulate the pH of the amplification reaction. A wide variety of buffers are known and commercially available. For example, morpholine buffers, such as 2-(N-morpholino)ethanesulfonic acid (MES), can be suitable for providing an effective pH range of about 5.0 to 6.5, imidazole buffers can be suitable for providing an effective pH range of about 6.2 to 7.8, and tris(hydroxymethyl)aminomethane (TRIS) buffers and certain piperazine buffers such as N-(2-hydroxyethyl)piperazine-N′-(2-ethanesulfonic acid) (HEPES) can be suitable for providing an effective pH range of about 7.0 to 9.0. The buffer can affect the activity and fidelity of nucleic acid amplifying enzymes, such as polymerases. For certain embodiments, the buffer is selected from at least one buffer which can regulate the pH in the range of 7.5 to 8.5. For certain of these embodiments, the buffer is a TRIS-based buffer. For certain of these embodiments, the buffer is selected from the group consisting of at least one of TRIS-EDTA, TRIS buffered saline, TRIS acetate-EDTA, and TRIS borate-EDTA. Other materials can be included with these buffers, such as surfactants and detergents, for example, CHAPS or a surfactant described below. The buffers may be free of RNase and DNase.

Salts can affect the activity of nucleic acid amplifying enzymes. For example, free magnesium ions are necessary for certain polymerases, such as Taq DNA polymerase, to be active. In another example, in the presence of manganese ions, Tfl DNA polymerase and Tth DNA polymerase can catalyze the polymerization of nucleotides into DNA, using RNA as a template. In a further example, the presence of certain salts, such as potassium chloride, can increase the activity of certain polymerases such as Taq DNA polymerase. For certain embodiments, the salt is selected from the group consisting of at least one of magnesium, manganese, zinc, sodium, and potassium salts. For certain of these embodiments, the salt is at least one of magnesium chloride, manganese chloride, zinc sulfate, zinc acetate, sodium chloride, and potassium chloride. For certain of these embodiments, the salt is magnesium chloride.

Bovine Serum Albumin can be used to stabilize the enzyme during nucleic acid amplification; dimethyl sulfoxide (DMSO) can be used to inhibit the formation of secondary structures in the DNA template; glycerol can improve the amplification process, can be used as a preservative as wells as other sugars (e.g., trehalose and sorbitol), and can stabilize enzymes such as polymerases; ethylenediaminetetraacectic acid (EDTA) and ethylene glycol-bis(2-aminoethylether)-N,N,N′N′-tetraacetic acid (EGTA) can be used as metal ion chelators and also to inactivate metal-binding enzymes (RNases) that may damage the reaction.

Amplification reactions that can be carried out with the methods, devices, and kits of the present invention are well known to those skilled in the art and include, for example, Strand Displacement Amplification (SDA), Polymerase Chain Reaction (PCR), Ligase Chain Reaction (LCR), Transcription Mediated Amplification (TMA), Nucleic Acid Sequence Based Amplification (NASBA), as well as bDNA, and INVADER techniques. One or more of such techniques can be used in series in the methods of the present invention. For example, methods described herein can further include transferring the amplificons to a different chamber that includes an amplification reagent for a second type of amplification reaction, wherein the amplification reagent includes one or more primers, one or more probes, and one or more enzymes for the second type of amplification reaction.

In a specific example, target nucleic acids (DNA or RNA) can be amplified with PCR or RT-PCR using a pair of primers (of which one contains a promoter sequence at its 5′ end) and a bacteriophage RNA polymerase (e.g., T7, SP6, or T3 RNA polymerase). PCR or RT-PCR generates double-stranded amplicons of which each contains a functional double-stranded bateriophage promoter at one of its ends. After this initial amplification with PCR or RT-PCR, the amplicons can be transferred to a second amplification chamber (or the bacteriophage RNA polymerase, ribonucleotides, molecular probes (e.g., Molecular beacons, Torches) and other required reagents can be introduced into the first amplification chamber) for in vitro transcription. In this second amplification step the PCR or RT-PCR amplicons are used as templates to generate RNA molecules which are detected by the molecular probes. Thus, in the initial amplification step, probes are not required.

In certain embodiments, the various reagents (e.g., functionalized support material, lysing reagent (which can be in a lysis buffer), neutralization reagent, amplification primers, probes, and enzymes) used throughout the methods of the present invention are added to process chambers during processing. For example, the step of contacting the nucleic acid-containing material with a lysing reagent includes adding the lysing reagent (which can be in a lysis buffer) to the process chamber after the nucleic acid-containing material is in the process chamber. As another example, a neutralization reagent can be added to a lysed sample, which may or may not include the functionalized support material.

Alternatively and preferably, the process chambers include the various reagents (e.g., functionalized support material, lysing reagent (which can be in a lysis buffer), neutralization reagent, amplification primers, probes, and enzymes) in dry form in one or more process chambers prior to use. For example, the lysing reagent (which can be in a lysis buffer) and amplification primers and probes are present in the process chambers of the process array in dry form prior to delivering sample material. The process chamber to which the sample material is delivered can contain the functionalized support material (preferably, nonspecific functionalized support material) and the lysing reagent (which can be in a lysis buffer) in dry form, such that contacting the nucleic acid-containing material with a lysing reagent and functionalized support material occurs simultaneously.

If separation between reagents is desired, such dried reagents can be in different chambers (e.g., lysing reagent (which can be in a lysis buffer) and functionalized support material (preferably, nonspecific functionalized support material), or basic lysing reagent (which can be in a lysis buffer) and neutralization reagent) or in the same chamber in different spatially separated portions of the chamber (e.g., primers and enzyme). Alternatively, one or more mixing chambers that are separated from a process chamber can be incorporated into the device with different reagents dried down in each of the mixing chambers and process chamber.

Alternatively, the process chamber to which the sample material is delivered contains the functionalized support material (preferably, nonspecific functionalized support material) in dry form and contacting the nucleic acid-containing material with a lysing reagent (which can be in a lysis buffer) and functionalized support material in the process chamber includes: contacting the nucleic acid-containing material first with the functionalized support material (preferably, nonspecific functionalized support material) under conditions effective to capture at least a portion of the nucleic acid-containing material; and subsequently adding the lysing reagent (which can be in the form of a lysis buffer) to the process chamber containing the functionalized support material and captured nucleic acid-containing material under conditions effective to lyse at least a portion of the nucleic acid-containing material, release nucleic acid, and capture at least a portion of the nucleic acid by the same functionalized support material (preferably, nonspecific functionalized support material).

Thus, various reagents can be dried down in the device prior to use, or they can be added during various stages of the process to various process chambers. If they are dried down in the device prior to use, they can be resuspended in water or a buffer such as a wash buffer, for example, and then added to various process chambers within the device during processing, although this is not required.

For certain embodiments, the methods of the present invention can include the use of an internal control. For example, the process chamber to which the sample material is delivered can contain an internal control; the sample material can include an internal control; and/or any of the reagents used in the lysing process (e.g., lysing reagent and/or neutralization reagent), including the functionalized support material, can be combined with an internal control. The internal control can include, for example, nucleic acid and/or cells from a known microorganism. For an effective internal control, the nucleic acid is distinct enough in structure from the nucleic acid of interest that it does not react with the primers and/or probes, but the nucleic acid is close enough in structure to be captured by the functionalized support material (preferably, nonspecific functionalized support material). The internal control can be used to monitor reagent integrity as well as inhibition from the sample material or specimen. Linearized plasmid DNA control is typically used as a nucleic acid internal control.

For certain embodiments, preferably, the methods of the present invention include detecting the nucleic acid, preferably the amplified nucleic acid. Detecting nucleic acid can occur with the nucleic acid still attached to the functionalized support material (preferably, nonspecific functionalized support material). Detecting amplified nucleic acid can include real-time detection of the amplification products during processing while the amplification products are still in the device. Alternatively, detecting amplified nucleic acid can include end-point detection where the amplification products are detected outside of the device. Detection can include fluorescence, chemiluminescence, bioluminescence, and the like.

For certain embodiments, the methods of the present invention include transferring the amplification products to a multi-array detection assay, which may or may not be included within a device of the present invention. For example, a multi-array detection assay can include a panel assay wherein gram positive and gram negative bacteria can be detected individually or in groups. Alternatively, the amplification can occur in a multi-array amplification system (e.g., a panel of chambers wherein each chamber includes primer and probe sets for a specific gene or interest). The multi-array amplification and multi-array detection assays can be used separately or in combination. Such multi-array detection systems are commonly carried out to detect individual bacteria or groups of bacteria in sepsis panels for blood.

For certain embodiments, contacting the nucleic acid-containing material with a lysing reagent (which can be included in a lysis buffer) and contacting the released nucleic acid with functionalized support material (preferably, nonspecific functionalized support material) occur in the same process chamber. For example, in an exemplary embodiment, the device includes a lysing reagent (which can be in a lysis buffer, and can include an enzymatic lysing reagent or a basic lysing reagent and an optional neutralization reagent, for example) and functionalized support material (preferably, nonspecific functionalized support material), both in dry form in the same process chamber. Upon delivery of the nucleic acid-containing material to the process chamber, lysis typically takes place with immediate capture of the released nucleic acid.

As mentioned above, if spatial separation between reagents is desired, such dried reagents can be in different chambers or in the same chamber in different spatially separated portions of the chamber. For example, in certain embodiments, contacting the nucleic acid-containing material with a lysing reagent (which can be included in a lysis buffer) and contacting the released nucleic acid with functionalized support material (preferably, nonspecific functionalized support material) can occur in different process chambers. Alternatively, in certain embodiments, contacting the nucleic acid-containing material with a lysing reagent (which can be included in a lysis buffer) and contacting the released nucleic acid with functionalized support material (preferably, nonspecific functionalized support material) occur in different portions of the same process chamber. This can be accomplished by spotting the reagents in different regions of the same chamber (e.g., the lysing reagent, which can be included in a lysis buffer, and functionalized support material are dried in “spots” in different regions of the same chamber) analogous to that described in WO 2005/118849). This can be supplemented with reagents having different dissolution rates in the different spots. Alternatively, one or more mixing chambers that are separated from a process chamber, but in fluid communication through mixing ports, can be incorporated into the device with different reagents dried down in two or more of the mixing and process chambers. If desired, a valve may be located between the mixing chambers and process chamber for control of the flow of materials therebetween. Such devices are described, for example, in WO 2005/061084.

Spatial separation of reagents may be desired if sequential process steps are desired (e.g., neutralization following alkaline lysis). It may be advantageous if the reagents are not stable together during the drying process (e.g., certain primers can dimerize in the presence of other amplification reagents upon dry down).

In one exemplary embodiment, a device can include a process chamber that includes a dried alkaline lysing reagent (which can be in a lysis buffer) and dried functionalized support material and a mixing chamber in fluid communication with the process chamber, wherein the mixing chamber includes a dried neutralization reagent for neutralization of the mixture subsequent to lysis. A sample can be initially introduced into the process chamber for lysis, and then the mixture (lysed sample material with functionalized support material including bound and unbound nucleic acid) introduced into the mixing chamber to neutralize the mixture and enhance binding of the nucleic acid to the functionalized support material. Alternatively, a wash buffer can be used to resuspend the reagent(s) in the mixing chamber and add them to the process chamber.

In one embodiment, a device can include a process chamber that includes a dried alkaline lysing reagent (which can be included in a lysis buffer) and a mixing chamber in fluid communication with the process chamber, wherein the mixing chamber includes dried functionalized support material and a dried neutralization reagent for neutralization of the mixture subsequent to lysis. A sample can be initially introduced into the process chamber for lysis, and then the lysed sample material introduced into the mixing chamber to neutralize the mixture and allow the nucleic acid to bind to the functionalized support material. Alternatively, a wash buffer can be used to resuspend the reagent(s) in the mixing chamber and add them to the process chamber.

In another embodiment, a device can include a process chamber that includes a dried alkaline lysing reagent (which can be included in a lysis buffer) and two separate mixing chambers in fluid communication with the process chamber, wherein one mixing chamber includes dried functionalized support material and one mixing chamber includes a dried neutralization reagent. A sample can be initially introduced into the process chamber for lysis, then the lysed sample material introduced into the mixing chamber to neutralize the mixture, and subsequently the neutralized lysed mixture introduced into the other mixing chamber to allow the nucleic acid to bind to the functionalized support material. Alternatively, a wash buffer can be used to resuspend the reagents in the mixing chambers and add them to the process chamber.

In another embodiment, a device can include a process chamber that includes a dried functionalized support material and two separate mixing chambers in fluid communication with the process chamber, wherein one mixing chamber includes dried alkaline lysing reagent (which can be included in a lysis buffer) and one mixing chamber includes a dried neutralization reagent. A sample can be initially introduced into the process chamber for mixing with the functionalized support material, then the sample material with functionalized support material introduced into the mixing chamber to lyse the sample material, and then the mixture (lysed sample material with functionalized support material including bound and unbound nucleic acid) introduced into the mixing chamber to neutralize the mixture and enhance binding of the nucleic acid to the functionalized support material. Alternatively, a wash buffer can be used to resuspend the reagents in the mixing chambers and add them to the process chamber.

In another embodiment, a device can include a process chamber that includes a dried functionalized support material and a mixing chamber in fluid communication with the process chamber, wherein the mixing chamber includes a dried enzymatic lysing reagent (which can be included in a lysis buffer). A sample can be initially introduced into the process chamber for mixing with the functionalized support material, and then the sample material with functionalized support material introduced into the mixing chamber to lyse the sample material and allow the nucleic acid to bind to the functionalized support material.

Each of the reagents can be supplemented with, for example, polymers or carbohydrates, to provide different dissolution rates, particularly if they are dried in different spots in one chamber. For certain embodiments, the reagent can include at least one of a water soluble polymer and a carbohydrate. The water soluble polymer and/or carbohydrate can provide a matrix that can hold or contain at least one reagent. The water soluble polymer and/or carbohydrate can also increase adhesion of the reagent. For certain of these embodiments, the water soluble polymer is selected from the group consisting of poly(ethylene glycol), poly(vinyl alcohol), partially hydrolyzed poly(vinyl alcohol), polyvinylpyrrolidone, poly(1-vinylpyrrolidone-co-2-dimethylaminoethylmethacrylate), poly(1-vinylpyrrolidone-co-vinyl acetate), and a combination thereof. For certain of these embodiments, the water soluble polymer is selected from the group consisting of poly(vinyl alcohol), poly(vinyl alcohol acetate), polyvinylpyrrolidone, and a combination thereof. For certain of these embodiments, the water soluble polymer is poly(vinyl alcohol) which is at least 80% hydrolyzed. For certain of these embodiments, the poly(vinyl alcohol) is at least 90% hydrolyzed and has a weight average molecular weight of about 30,000 to about 70,000. For certain of these embodiments, the carbohydrate is selected from the group consisting of sucrose, trehalose, mannitol, sorbitol, raffinose, stachyose, melezitose, dextrose, maltose, dextran, cellobiose, pectin, hydroxyethylcellulose, hydroxypropylcellulose, hydroxypropylmethylcellulose, guar gum, locust gum, gum arabic, xanthan gum, ficoll, a poly(ethylene oxide)-poly(propylene oxide) copolymer with a hydrophilic/lipophilic balance of greater than 7, preferably greater than 9, more preferably about 12, a cyclodextrin, α-cyclodextrin, starch, pullulan, alginates, gelatins, and carrageenans. For certain of these embodiments, the carbohydrate is selected from the group consisting of sucrose, dextran, trehalose, pullulan, α-cyclodextrin, mannitol, sorbitol, and a combination thereof. For certain embodiments, the carbohydrate is a sugar. For certain of these embodiments, the reagent layer includes the water soluble polymer or any one of the above embodiments of the water soluble polymer. For certain of these embodiments, the reagent layer does not include the carbohydrate. Alternatively, for certain of these embodiments, including any one of the above embodiments which includes at least one of a water soluble polymer and a carbohydrate, the reagent includes the carbohydrate or any one of the above embodiments of the carbohydrate. For certain of these embodiments, the reagent does not include the water soluble polymer.

### Devices and Kits

The present invention provides a kit, which can include a functionalized support phase material in a microfluidic device that includes a process array, a lysing reagent (which can be included in a lysis buffer), optionally other reagents (e.g., wash buffer, neutralization buffer, sample prep and amplification controls), and instructions for amplifying nucleic acid. Preferably, prior to placing any reagents in the device, the device is cleaned to remove any undesired reagents, particularly RNase and DNase inhibitors.

Other components that could be included within kits of the present invention include conventional reagents such as wash solutions (e.g., wash buffers), elution buffers, external positive or negative controls, and the like. Other components that could be included within kits of the present invention include conventional equipment such as spin columns, cartridges, 96-well filter plates, syringe filters, collection units, syringes, sample collection devices (e.g., swabs), and the like.

The kits typically include packaging material, which refers to one or more physical structures used to house the contents of the kit. The packaging material can be constructed by well-known methods, preferably to provide a sterile, contaminant-free environment. The packaging material may have a label that indicates the contents of the kit. In addition, the kit contains instructions indicating how the materials within the kit are employed. As used herein, the term “package” refers to a solid matrix or material such as glass, plastic, paper, foil, and the like. The packaging material may also include a dessicant or may be purged with nitrogen for maintaining sample integrity.

“Instructions” typically include a tangible expression describing the various methods of the present invention, including lysing conditions such as lysing reagent type and concentration, the relative amounts of reagent and sample to be admixed, maintenance time periods for reagent/sample admixtures, temperature, buffer conditions, and the like.

Devices of the present invention include a process array that includes process chambers defining volumes for containing sample material or portions thereof (i.e., nucleic acid-containing material, nucleic acid) and a conduit (forming one or more channels) connecting the process chambers. The device may provide uniform and accurate temperature control of one or more of the chambers. The device provides a conduit defining one or more channels between chambers, for example, such that sample preparation may take place in one or more chambers, and nucleic acid amplification and optional detection may take place in one or more other chambers. For certain embodiments, including any one of the above embodiments which include the device for processing sample material, the device for processing sample material is a microfluidic device. Some examples of microfluidic devices are described in U.S. Publication Numbers 2002/0064885 (Bedingham et al.); US2002/0048533 (Bedingham et al.); US2002/0047003 (Bedingham et al.); and US2003/138779 (Parthasarathy et al.); as well as U.S. Pat. Nos. 6,627,159; 6,720,187; 6,734,401; 6,814,935; 6,987,253; 7,026,168; and 7,164,107.

Preferred devices useful in methods of the present invention include microfluidic devices. These typically employ a body structure that has an integrated microfluidic channel network disposed therein. In preferred aspects, the body structure of the microfluidic devices include an aggregation of two or more separate layers which, when appropriately mated or joined together, form the microfluidic device of the invention, e.g., containing the channels and/or chambers described herein. Typically, useful microfluidic devices include a top portion, a bottom portion, and an interior portion, wherein the interior portion substantially defines the channels and chambers of the device. Typically, the chambers include valves (e.g., valve septums) and are referred to as valved chambers.

A particularly preferred device for certain embodiments herein is referred to as a variable valve device and is disclosed in U.S. Patent Publication No. 2005/0130177. In this variable valve device, the valve structures allow for removal of selected portions of the sample material located within the process chamber (i.e., the variable valved process chamber). Removal of the selected portions is achieved by forming an opening in a valve septum at a desired location.

The valve septums are preferably large enough to allow for adjustment of the location of the opening based on the characteristics of the sample material in the process chamber. If the sample processing device is rotated after the opening is formed, the selected portion of the material located closer to the axis of rotation exits the process chamber through the opening. The remainder of the sample material cannot exit through the opening because it is located farther from the axis of rotation than the opening.

The openings in the valve septum may be formed at locations based on one or more characteristics of the sample material detected within the process chamber. It may be preferred that the process chambers include detection windows that transmit light into and/or out of the process chamber. Detected characteristics of the sample material may include, e.g., the free surface of the sample material (indicative of the volume of sample material in the process chamber). Forming an opening in the valve septum at a selected distance radially outward of the free surface can provide the ability to remove a selected volume of the sample material from the process chamber.

For sample materials that can be separated into various components, e.g., whole blood, rotation of the sample processing device may result in separation of the plasma and red blood cell components, thus allowing for selective removal of the components to, e.g., different process chambers.

In some embodiments, it may be possible to remove selected aliquots of the sample material by forming openings at selected locations in one or more valve septums. The selected aliquot volume can be determined based on the radial distance between the openings (measured relative to the axis of rotation) and the cross-sectional area of the process chamber between the opening.

The openings in the valve septums are preferably formed in the absence of physical contact, e.g., through laser ablation, focused optical heating, etc. As a result, the openings can preferably be formed without piercing the outermost layers of the sample processing device, thus limiting the possibility of leakage of the sample material from the sample processing device.

In one aspect, the present invention uses a valved process chamber in a sample processing device (e.g., a microfluidic device), the valved process (e.g., heating, mixing, lysing, combining fluids) chamber including a process chamber having a process chamber volume located between opposing first and second major sides of the sample processing device, wherein the process chamber occupies a process chamber area in the sample processing device, and wherein the process chamber area has a length and a width transverse to the length, and further wherein the length is greater than the width. The variable valved process chamber also includes a valve chamber located within the process chamber area, the valve chamber located between the process chamber volume and the second major side of the sample processing device, wherein the valve chamber is isolated from the process chamber by a valve septum separating the valve chamber and the process chamber, and wherein a portion of the process chamber volume lies between the valve septum and a first major side of the sample processing device. A detection window is located within the process chamber area, wherein the detection window is transmissive to selected electromagnetic energy directed into and/or out of the process chamber volume.

In another aspect, the present invention provides a method that allows for the selective removal of a portion of a sample from a variable valved process chamber. The method includes providing a sample processing device (e.g., a microfluidic device) as described above, providing sample material in the process chamber; detecting a characteristic of the sample material in the process chamber through the detection window; and forming an opening in the valve septum at a selected location along the length of the process chamber, wherein the selected location is correlated to the detected characteristic of the sample material. The method also includes moving only a portion of the sample material from the process chamber into the valve chamber through the opening formed in the valve septum.

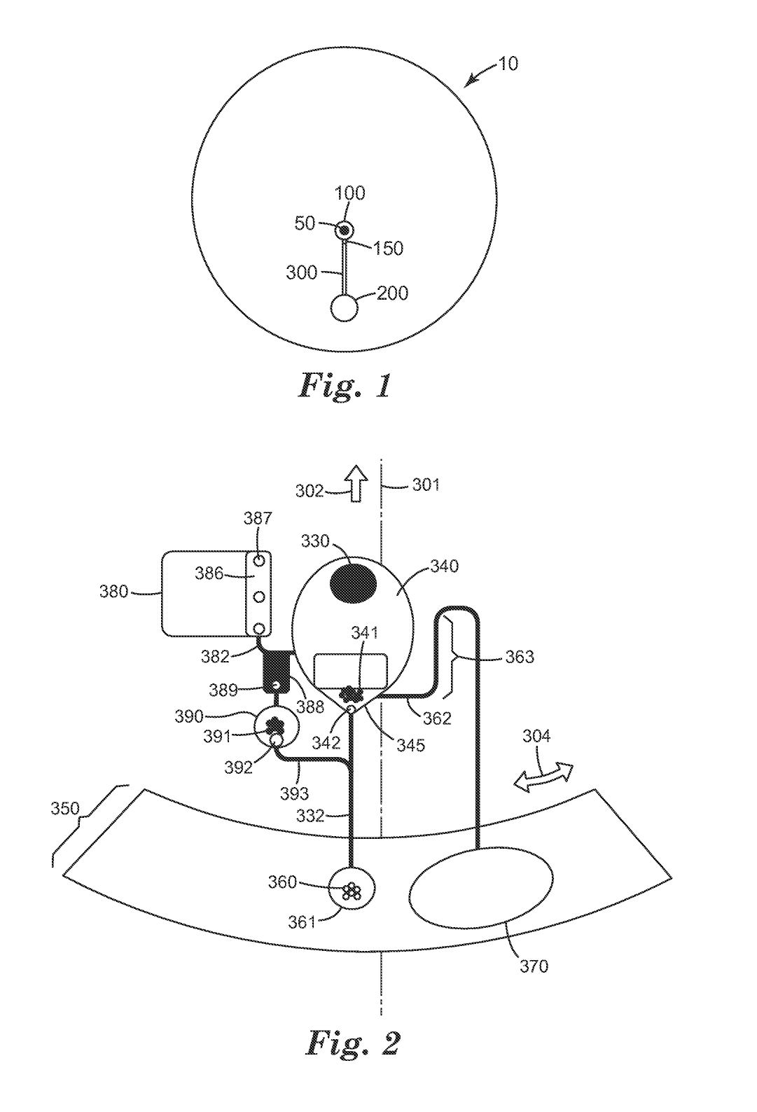

One illustrative device for processing sample material is the microfluidic device depicted in FIG. 1. The device 10 can be in the shape of a circular disc as illustrated in FIG. 1, although other shapes can be used. Preferred shapes are those that can be rotated. The device 10 of FIG. 1 comprises a first chamber 100 and a second chamber 200 which can be in fluid communication with the first chamber 100 via channel 300. The shape of chambers 100 and 200 can be circular as illustrated in FIG. 1, although other shapes, for example, oval, tear-drop, triangular, and many others can be used. FIG. 1 illustrates one combination of chamber 100 and chamber 200, but it is to be understood that a plurality of such combinations can be included in device 10 and may be desirable for simultaneously processing a plurality of samples.

The device 10 illustrated in FIG. 1 includes the immobilized-metal support material 50 in chamber 100 and a lysis reagent. The functionalized support material 50 can be a plurality of magnetic or non-magnetic particles, illustrated in FIG. 1. Sample preparation such as binding nucleic acid-containing material, lysing, binding nucleic acid, washing, and the like, can be carried out in chamber 100 prior to moving functionalized support material having nucleic acid attached thereto in chamber 100 through channel 300 and into chamber 200.

Moving material from chamber 100 to chamber 200 can be carried out, for example, by applying a sufficient g-force to the functionalized support material to force the material through channel 300 and into chamber 200. Alternatively, a pressure differential can be applied to channel 300, for example, by reducing the pressure in chamber 200, by increasing the pressure in chamber 100, or both, thereby causing material in chamber 100 to move through channel 300 and into chamber 200. Chamber 100 or channel 300 can be equipped with optional valve 150. Valve 150 can be fabricated to open by exposure to a sufficient g-force, by melting, by vaporizing, or the like. For example, the valve can be fabricated in the form of a septum in which an opening can be formed through laser ablation, focused optical heating, or similar means. Such valves are described, for example in U.S. Pat. No. 7,322,254 B1, issued Jan. 29, 2008 (Bedingham et al.) and U.S. Patent Application Publication No. 2005/0142571 A1 (Parthasarathy et al.).

For certain embodiments, chamber 200 includes an amplification reagent. Nucleic acid amplification may include, for example, producing a complementary nucleic acid or a portion thereof in sufficient numbers for detection. The amplification reagent can include one or more primers, optionally one or more probes, optionally one or more amplification enzymes, and other optional components.

Although not shown in FIG. 1, chambers 100 and 200 and channel 300 can be in fluid communication with other chambers, channels, reservoirs, and/or the like. These can be used to facilitate supplying or removing various reagents, sample material(s), or a component(s) of a sample material to or from chambers 100 or 200 as needed. For example, sample materials, lysis reagents, digestion reagents, wash buffers, binding buffers, and/or the like can be supplied to and/or removed from chamber 100, and primers, nucleotide triphosphates, amplification enzymes, probes, buffers, and/or the like can be supplied to chamber 200. Individual reagents or combinations of reagents can be placed in different chambers, whether included in the device 10 or in any embodiment of the device described herein, to subsequently contact the reagents with the sample material or a component of the sample material as desired.

Various valving mechanisms may be used in the present invention to control movement of materials through the array of chambers and passageways or channels present on the device. A valve can opened by forming a void in the a portion of the chamber wall or through a conduit connecting the chamber to another chamber or a channel. The void may be formed by electromagnetic energy of any suitable wavelength. It may be preferred that laser energy of a suitable wavelength be used. The valve may be selectively opened, using, for example, laser energy, while the device is rotating during sample processing. Valves formed with lasers are further described in the literature including U.S. Pat. No. 6,617,136.

Although described in connection with rotating devices in which centrifugal force generated by rotation can be used to move fluids (e.g., gases and/or liquids) within the conduits and chambers, the methods and devices of the present invention may also be used in connection with gravitational forces (actual or induced) to move fluids, in which case the device itself need not be rotated. It should, however, be understood that methods and devices of the present invention may, in some instances, rely on gravitation force and centrifugal force to move fluids through the process arrays (with the gravitational and centrifugal forces acting simultaneously or at different times). Such transfer methods (thermopipetting methods) can be used anywhere in the methods of the present invention, but preferably, they are used in connection with washing, particularly if multiple washings of the functionalized support material in the same process chamber are desired.

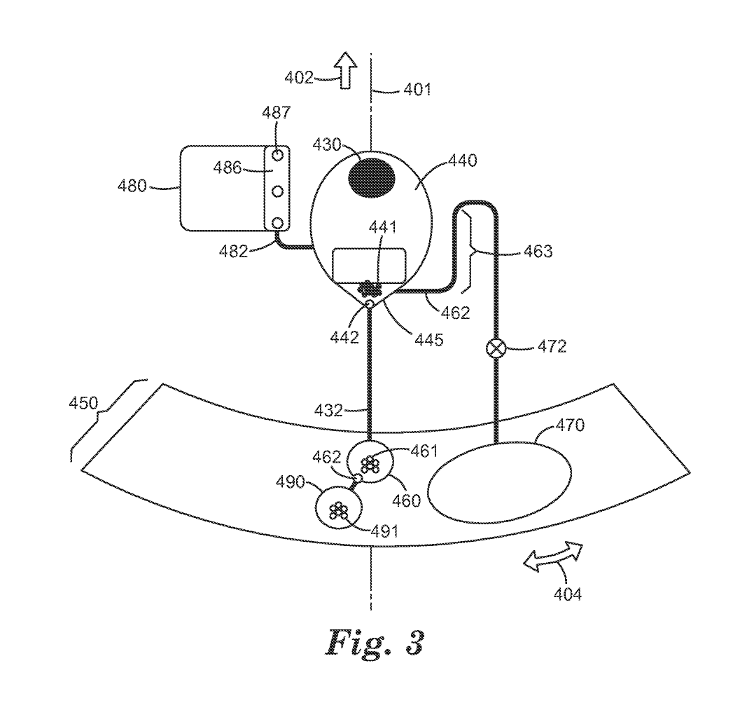

An exemplary process array that may be provided in a device according to the present invention is depicted in FIG. 2. Such methods and devices are described in greater detail in U.S. Patent Application Ser. No. 60/871,611, filed on Dec. 22, 2006, and U.S. patent application Ser. No. 11/962,703 (Attorney Docket No. 62471US005) and WO US2007/088532 (Attorney Docket No. 62471WO003), filed Dec. 21, 2007, both entitled “THERMAL TRANSFER METHODS AND STRUCTURES FOR MICROFLUIDIC SYSTEMS.”

The exemplary process array of FIG. 2 is preferably provided in a device designed for rotation about an axis of rotation and may be located on or near radius 301 in the direction of arrow 302. When rotated about the axis of rotation, the features of the process array will travel generally in the directions indicated by arcuate arrow 304. Alternatively, the process array of FIG. 2 may be used in a non-rotating gravity based device in which the arrow 302 is indicative of the upstream direction, i.e., is opposite of the direction of the gravitational forces acting on the process array (where the direction of the gravitational forces is the downstream direction).

The exemplary process array includes a first process chamber 340 that connects to a second process chamber 360 through a conduit 332. The first process chamber 340 and the second process chamber 360 may preferably be arranged on the processing device to define an upstream direction and a downstream direction. The upstream direction is the direction when moving from the second process chamber 360 towards the first process chamber 340 (in the general direction indicated by the arrow 302). The downstream direction is the direction when moving from the first process chamber 340 towards the second process chamber 360. It may be preferred that the upstream and downstream directions be substantially radially aligned with the center of the processing device in which this array is located in the case of a rotating system or aligned with gravitational forces in gravitational system.

The first process chamber 340 preferably includes a single-use valve 342 that preferably prevents fluids from passing into the conduit 332 until opened. The valve 342 may take the form of an overhanging valve lip. The first process chamber 340 includes a radially distal or downstream end 345 into which materials move when the processing device containing the process array is rotated about the axis of rotation or is subject to gravitational forces. The first process chamber 340 also preferably includes a loading structure 330 through which analyte (e.g., functionalized support material (preferably, nonspecific functionalized support material) with nucleic acid optionally attached thereto) may be introduced into the first process chamber 340. In the depicted embodiment, the first process chamber 340 also includes optional reagents 341 that may be used in the processing.

The second process chamber 360 may preferably be located in a region 350 of the processing device that is thermally controlled, e.g., can be heated and/or cooled to change the temperature of analyte or other materials located in the second process chamber 360. The region 350 may preferably be in the form of an annular ring (an arcuate portion of which is depicted in FIG. 2) if the processing device is in the form of a circular disc. As a result, the second process chamber 360 may be used to process analytes that require thermal control, e.g., isothermal processes, processes requiring thermal cycling between two or more different temperatures (e.g., PCR, etc.), etc. The depicted second process chamber 360 includes optional reagents 361 that may be used in connection with the processing.

The process array depicted in FIG. 2 also includes a thermal transfer structure (which can include a waste chamber) to assist with thermal transfer of fluids (which can be gases and/or liquids) through the first process chamber 340. In the exemplary embodiment depicted in FIG. 2, the thermal transfer structure includes a thermal drive chamber 370 (preferably, a waste chamber) in fluid communication with the first process chamber 340 through a transfer conduit 362. The thermal drive chamber 370 may preferably be positioned within thermally-controlled region 350 on the processing device.

In use, the depicted thermal transfer structure can be used to transfer fluids from the first process chamber 340 into the transfer conduit 362 and the drive chamber 370. The thermal drive chamber 370 may, therefore, serve as a reservoir for fluids removed from the first process chamber 340 (as well as providing the resident fluid that is used to perform the thermal transfer). It may be preferred that the thermal drive chamber 370 have a volume large enough to accept multiple fluid transfers from the first process chamber 340. The volume of the thermal drive chamber 370 may, e.g., preferably be equal to or greater than the volume of the first process chamber 340.

Thermal transfer of analytes (or other fluids) may be accomplished by use of transfer conduit 362 preferably including fluid trap 363 in which a portion of the transfer conduit 362 travels in the upstream direction between the transfer port (where the transfer conduit connects with the first process chamber 340) and the thermal drive chamber 370 (when moving from the first chamber 340 towards the thermal drive chamber 370). That fluid trap 363 effectively prevents fluids from moving out of the first chamber 340 to the thermal drive chamber 370 by rotation of the device containing the process array or under the influence of gravitational forces.

It may be preferred that the fluid trap 363 reach a level that is radially above (i.e., closer to the axis of rotation or upstream of) the levels of any fluids located in chamber 340 such that rotation of the device (or gravity) alone will not drive the analyte in the chamber 340 past the fluid trap 363 and into thermal drive chamber 370. The height of the fluid trap 363 may vary depending on a variety of factors including, e.g., the maximum height of the fluids in the chamber 340, the size of the transfer conduit 362, the hydrophobicity/hydrophilicity of the materials used to construct the process array, etc.

It may be preferred that the fluid trap 363 reach a height (measured in the upstream direction from the radially distal or downstream end 345 of the chamber 340) that is at least 25% or more of the height of the chamber 340 (where the height of the chamber 340 is measured from its radially distal or downstream end 345 to its radially proximal or upstream end—i.e., the end located closest to the axis of rotation). Alternatively, the fluid trap 363 in the transfer conduit 362 may preferably reach a height that is at least 50% or more of the height of the chamber 340. In still another alternative, the fluid trap 363 in the transfer conduit 362 may preferably reach a height that is at least 75% or more of the height of the chamber 340. In yet another alternative, the fluid trap 363 in the transfer conduit 362 may preferably reach a height that is at least 90% or more of the height of the chamber 340.

It may be preferred that any fluids to be transferred out of the first process chamber 340 be located in chamber 340 at or upstream of (i.e., closer to the axis of rotation) the transfer port at which the transfer conduit 362 connects to the first process chamber 340. If the processing device is rotating about an axis of rotation as discussed above while the valve 342 is closed, centrifugal force will drive fluids in the first process chamber 340 towards the radially distal or downstream end 345 of the first process chamber 340 so that the transfer port at which the transfer conduit 362 connects to the first process chamber 340 is covered by the fluid. If the system is not rotating, gravitational forces may be used to move fluids towards the downstream end 345 of the first process chamber 340. The result is that any resident fluid forced into the first process chamber 340 from the transfer conduit 362 preferably passes through the analyte in the second process chamber 340.

Other optional features depicted in the exemplary process array of FIG. 2 include a third chamber 380 that can be placed in fluid communication with the first process chamber 340 through a conduit 382. The conduit 382 is depicted as connecting with the first process chamber 340 at an intermediate point of the second process chamber 340. The conduit 382 may, alternatively connect to the first process chamber 340 at any selected location along the height of the process chamber 340 (where the height of the chamber is determined between its upstream and downstream ends).

Another optional feature depicted in connection with the exemplary process array of FIG. 2 is a single-use valve structure 386 used to control the flow of fluid from the third chamber 380 into the conduit 382. In the depicted process array, the valve structure takes the form of a valve lip that extends into the volume of the third chamber 380, with the valve lip including a valve septum through which openings 387 may be formed to allow fluid to flow from the third chamber 380 into the conduit 382.

In addition to the third chamber 380, the process array may also include a subchamber 388 in which fluid from the third chamber 380 may collect during use. The fluid that collects in subchamber 388 may be delivered into an intermediate chamber 390. Control over delivery of fluid to the intermediate chamber 390 may be provided by single-use valve 389.

For example, when valve 389 is opened (after subchamber 388 is filled with a fluid), fluid from subchamber 388 can enter intermediate chamber 390 which may preferably contain one or more reagents 391. The reagents 391 may preferably interact with or be taken up by the fluid from the subchamber 388. At a selected time, a single-use valve 392 in intermediate chamber 390 may be opened. When the valve 392 is opened, the fluids in the intermediate chamber may be delivered to the second process chamber 360 through conduit 393 which is in fluid communication with process conduit 332.

Still another exemplary process array that may be provided in a processing device according to the present invention is depicted in FIG. 3. The exemplary process array of FIG. 3 is preferably provided in a processing device designed for rotation about an axis of rotation and may be located on or near radius 401 in the direction of arrow 402. When rotated about the axis of rotation, the features of the process array will travel generally in the directions indicated by arcuate arrow 404. Alternatively, the process array of FIG. 3 may be used in a non-rotating gravity based device in which the arrow 402 is indicative of the upstream direction, i.e., is opposite of the direction of the gravitational forces acting on the process array (where the direction of the gravitational forces is the downstream direction).

The exemplary process array of FIG. 3 is similar in many respects to the process array depicted in FIG. 2 and includes features such as a first process chamber 440, reagents 441, loading structure 430, single-use valve 442 and downstream end 445 that are found in the first process chamber 340. In addition, the process array of FIG. 9 also includes a second process chamber 460 connected to the first process chamber 440 by a process conduit 432, as well as a third chamber 480 and valve structure 486 through which openings 487 may be formed to deliver fluids to the first process chamber 440 through conduit 482.

Also similar to the process array of FIG. 2, the process array of FIG. 3 also includes a transfer conduit 462 that connects a thermal drive chamber 470 to the first process chamber 440. Thermal drive chamber 470 is preferably located within a thermally-controlled region of the processing device in which the process array is located to provide the thermal control needed to effect thermal transfer in accordance with the principles of the present invention. The transfer conduit 462 include a fluid trap 463 to prevent movement of fluid from the first chamber 440 to the thermal drive chamber 470 through rotation of the processing device or gravitational forces alone.

An additional feature depicted in the process array of FIG. 3 is the valve 472 located along the transfer conduit 462. The valve 472 may be used to control activation of the thermal transfer function. For example, if the valve 472 is closed, heating or cooling of the resident fluid in the thermal drive chamber 470 will not function to pull or move fluids from the first process chamber 440. The exact location of the valve 472 is unimportant—it must merely be located between the first process chamber 440 and the thermal drive chamber 470. The valve 472 may be a single-use valve similar to those described herein.

Another difference between the process array of FIG. 3 and the process array of FIG. 2 is that the process array of FIG. 3 does not include the subchamber and intermediate chamber of the process array of FIG. 2. The process array of FIG. 3 does, however, include a third process chamber 490 located within the thermally-controlled region 450 as is second process chamber 460. Second chamber 460 includes optional reagents 461 located therein. The third process chamber 490 also includes optional reagents 491 located therein. The third process chamber 490 is also connected to the second process chamber 460 through single-use valve 462 and conduit 492. Rotation of the processing device in which the process array of FIG. 3 is located or gravitational forces will preferably move fluids from the second process chamber 460 to the third process chamber 490 where, as here, the third process chamber 490 is located downstream of the second process chamber 460.

Yet another exemplary process array is depicted in connection with FIG. 4 and illustrates another optional feature in process arrays of the present invention. The exemplary process array of FIG. 4 is preferably provided in a processing device designed for rotation about an axis of rotation and may be located on or near radius 501 in the direction of arrow 502. When rotated about the axis of rotation, the features of the process array will travel generally in the directions indicated by arcuate arrow 504. Alternatively, the process array of FIG. 4 may be used in a non-rotating gravity based device in which the arrow 502 is indicative of the upstream direction, i.e., is opposite of the direction of the gravitational forces acting on the process array (where the direction of the gravitational forces is the downstream direction).

The exemplary process array of FIG. 4 is similar in many respects to the process array depicted in FIGS. 2 and 3, and includes features such as a first process chamber 540, loading structure 530, single-use valve 542 and downstream end 545 that are found in the first process chamber 540. In addition, the process array of FIG. 4 also includes a second process chamber 560 connected to the first process chamber 540 by a process conduit 532.

Also similar to the process arrays of FIGS. 2 and 3, the process array of FIG. 4 includes a transfer conduit 562 that connects the first process chamber 540 to a pair of thermal drive chambers 570a and 570b. The transfer conduit 562 includes a fluid trap 563 after which the transfer conduit 562 splits into transfer conduits 562a and 562b. Both of the thermal drive chambers 570a and 570b are preferably located in the thermally-controlled region 550.

Each of the transfer conduits 562a and 562b may preferably include a valve 572a and 572b (respectively) to control flow of fluids into and out of the thermal drive chambers 570a and 570b. The valves 572a and 572b may preferably take the form of single-use valves as described herein. In some instances, one of the thermal drive chambers may not be isolated from the first process chamber 540 by a valve, with additional thermal drive chambers isolated using valves. Further, although only two thermal drive chambers are depicted in the process array of FIG. 4, three or more thermal drive chambers may be provided if so desired. In another variation, where multiple thermal drive chambers are provided, each thermal drive chamber may be connected to the process chamber 540 using a dedicated transfer conduit (in place of splitting the conduit 562 as depicted in FIG. 4).

The use of reagents in connection with the process arrays in processing devices of the present invention is optional, i.e., processing devices of the present invention may or may not include any reagents in the process array chambers. In another variation, some of the chambers in different process arrays may include a reagent, while others do not. In yet another variation, different chambers may contain different reagents. Further, the interiors of the chamber structures may be coated or otherwise processed to control the adhesion of reagents.

It is understood that an analyte, such as described in the foregoing embodiments, may be in the form of a fluid (e.g., a solution, etc.), or the analyte may be in the form of a solid or a semi-solid material carried in a fluid. The analyte may be entrained in the fluid, in solution within the fluid, etc. Furthermore, the term “analyte” may be used to refer to fluids in which a target analyte (i.e. the analyte sought to be processed) is not present. For example, wash fluids (e.g., saline, etc.) may be referred to as an analyte for the purposes of the present invention.

Exemplary embodiments using such thermal transfer structure are described below in embodiments 11-14 and 87-90, for example. In a typical method using such thermal transfer structure, washing and/or decanting a functionalized support material with nucleic acid attached thereto, for example can be accomplished by: optionally adding a wash solution to the process chamber comprising the functionalized support material with nucleic acid attached thereto; passing a first portion of the resident fluid (typically, gas) through a thermal transfer conduit into the process chamber comprising the functionalized support material with nucleic acid attached thereto by heating at least a portion of the resident fluid in the thermal drive chamber (typically, a waste chamber), wherein the thermal transfer conduit connects the thermal drive chamber to the process chamber comprising the functionalized support material with nucleic acid attached thereto, and wherein the volume of the resident fluid within the thermal transfer structure increases to force the first portion of the resident fluid into the process chamber comprising the functionalized support material with nucleic acid attached thereto; and subsequently cooling the heated resident fluid in the thermal transfer structure, wherein the volume of the resident fluid within the thermal transfer structure decreases such that at least a portion of the liquid present in the process chamber comprising the functionalized support material with nucleic acid attached thereto is drawn into the thermal drive chamber through the thermal transfer conduit. This method can result in simply decanting at least a portion of the liquid in the process chamber that contains the funtionalized support material, or it can involve washing the functionalized support material and decanting the wash solution. This can be carried out multiple times using multiple aliquots of wash solution, if desired. With each decanting and/or washing, preferably, a majority of the functionalized support material is left behind. This can be accomplished by packing the functionalized support material, for example, using a magnet.

### Exemplary Embodiments

Exemplary embodiments of the present invention include the following:

1. A method of amplifying nucleic acid, the method comprising:

providing a device comprising a process array that comprises process chambers defining volumes for containing sample material or portions thereof and a conduit connecting the process chambers;

delivering sample material comprising nucleic acid-containing material to a process chamber of the device;

optionally contacting the nucleic acid-containing material with nonspecific functionalized support material under conditions effective to capture at least a portion of the nucleic acid-containing material by the nonspecific functionalized support material in the process chamber;

optionally transferring the nonspecific functionalized support material and captured nucleic acid-containing material to a different process chamber;

contacting the nucleic acid-containing material, optionally attached to the nonspecific functionalized support material, with one or more lysing reagents (which can be included in a lysis buffer) under conditions effective to lyse at least a portion of the nucleic acid-containing material and release nucleic acid;

in the same or a different process chamber, contacting the released nucleic acid with nonspecific functionalized support material, which may be the same or different than the nonspecific functionalized support material optionally used to capture the nucleic acid-containing material, under conditions effective to capture at least a portion of the nucleic acid by the same or different nonspecific functionalized support material, wherein the nonspecific functionalized support material contacting the released nucleic acid comprises immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; wherein M is selected from the group consisting of zirconium, gallium, iron, aluminum, scandium, titanium, vanadium, yttrium, and a lanthanide; y is an integer from 3 to 6; and x is 1 or 2;

washing the nonspecific functionalized support material with nucleic acid attached thereto;

transferring the nonspecific functionalized support material with nucleic acid attached thereto to a different process chamber through the conduit by rotating the device;

before, during, or after transferring the nonspecific functionalized support material with nucleic acid attached thereto, contacting it with an amplification reagent, wherein the amplification reagent comprises one or more primers and optionally one or more probes;

optionally heating the nonspecific functionalized support material with nucleic acid attached thereto at a temperature effective to release and/or denature at least a portion of the captured nucleic acid in the presence of the amplification reagent; and

contacting the nucleic acid, which is optionally released and/or denatured from the nonspecific functionalized support material, and the amplification reagent with one or more amplification enzymes under conditions effective to amplify the nucleic acid to produce amplicons.

2. A method of amplifying nucleic acid, the method comprising:

providing sample material comprising nucleic acid-containing material;

contacting the nucleic acid-containing material with nonspecific functionalized support material under conditions effective to capture at least a portion of the nucleic acid-containing material by the nonspecific functionalized support material;

providing a device comprising a process array that comprises process chambers defining volumes for containing sample material or portions thereof and a conduit connecting the process chambers;

delivering nonspecific functionalized support material and captured nucleic acid-containing material to a process chamber of the device;

contacting the nucleic acid-containing material with one or more lysing reagents (which can be included in a lysis buffer) in the process chamber under conditions effective to lyse at least a portion of the nucleic acid-containing material and release nucleic acid;

in the same or a different process chamber, contacting the released nucleic acid with nonspecific functionalized support material, which may be the same or different than the nonspecific functionalized support material used to capture the nucleic acid-containing material, under conditions effective to capture at least a portion of the nucleic acid by the same or different nonspecific functionalized support material, wherein the nonspecific functionalized support material contacting the released nucleic acid comprises immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; wherein M is selected from the group consisting of zirconium, gallium, iron, aluminum, scandium, titanium, vanadium, yttrium, and a lanthanide; y is an integer from 3 to 6; and x is 1 or 2;

washing the nonspecific functionalized support material with nucleic acid attached thereto;

transferring the nonspecific functionalized support material with nucleic acid attached thereto to a different process chamber through the conduit by rotating the device;

before, during, or after transferring the nonspecific functionalized support material with nucleic acid attached thereto, contacting it with an amplification reagent, wherein the amplification reagent comprises one or more primers and optionally one or more probes;

optionally heating the nonspecific functionalized support material with nucleic acid attached thereto at a temperature effective to release and/or denature at least a portion of the captured nucleic acid in the presence of the amplification reagent; and

contacting the nucleic acid, which is optionally released and/or denatured from the nonspecific functionalized support material, and the amplification reagent with one or more amplification enzymes under conditions effective to amplify the nucleic acid to produce amplicons.

3. A method of amplifying nucleic acid, the method comprising:

providing a device comprising a process array that comprises process chambers defining volumes for containing sample material or portions thereof and a conduit connecting the process chambers;

delivering sample material comprising nucleic acid-containing material to a process chamber of the device;

optionally contacting the nucleic acid-containing material with nonspecific functionalized support material under conditions effective to capture at least a portion of the nucleic acid-containing material by the nonspecific functionalized support material in the process chamber;

optionally transferring the nonspecific functionalized support material and captured nucleic acid-containing material to a different process chamber;

contacting the nucleic acid-containing material, optionally attached to the nonspecific functionalized support material, with one or more lysing reagents (which can be included in a lysis buffer) under conditions effective to lyse at least a portion of the nucleic acid-containing material and release nucleic acid;

in the same or a different process chamber, contacting the released nucleic acid with nonspecific functionalized support material, which may be the same or different than the nonspecific functionalized support material optionally used to capture the nucleic acid-containing material, under conditions effective to capture at least a portion of the nucleic acid by the same or different nonspecific functionalized support material, wherein the nonspecific functionalized support material contacting the released nucleic acid comprises immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; wherein M is selected from the group consisting of zirconium, gallium, iron, aluminum, scandium, titanium, vanadium, yttrium, and a lanthanide; y is an integer from 3 to 6; and x is 1 or 2;

washing the nonspecific functionalized support material with nucleic acid attached thereto;

transferring the nonspecific functionalized support material with nucleic acid attached thereto to a different process chamber through the conduit by rotating the device;
wherein the nucleic acid contacts an amplification reagent located in the different process chamber;
wherein the amplification reagent comprises one or more primers, optionally one or more probes, and one or more amplification enzymes; and
wherein the one or more amplification enzymes are thermally stable at the temperature effective to release and/or denature the nucleic acid;

heating the nonspecific functionalized support material with nucleic acid attached thereto at a temperature effective to release and/or denature at least a portion of the nucleic acid in the presence of the amplification reagent; and

providing conditions effective to amplify the released and/or denatured nucleic acid to produce amplicons.

4. A method of amplifying nucleic acid, the method comprising:

providing sample material comprising nucleic acid-containing material;

contacting the nucleic acid-containing material with nonspecific functionalized support material under conditions effective to capture at least a portion of the nucleic acid-containing material by the nonspecific functionalized support material;

providing a device comprising a process array that comprises process chambers defining volumes for containing sample material or portions thereof and a conduit connecting the process chambers;

delivering nonspecific functionalized support material and captured nucleic acid-containing material to a process chamber of the device;

contacting the captured nucleic acid-containing material with one or more lysing reagents (which can be included in a lysis buffer) in the process chamber under conditions effective to lyse at least a portion of the nucleic acid-containing material and release nucleic acid;

in the same or a different process chamber, contacting the released nucleic acid with nonspecific functionalized support material, which may be the same or different than the nonspecific functionalized support material used to capture the nucleic acid-containing material, under conditions effective to capture at least a portion of the nucleic acid by the same or different nonspecific functionalized support material, wherein the nonspecific functionalized support material contacting the released nucleic acid comprises immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; wherein M is selected from the group consisting of zirconium, gallium, iron, aluminum, scandium, titanium, vanadium, yttrium, and a lanthanide; y is an integer from 3 to 6; and x is 1 or 2;

washing the nonspecific functionalized support material with nucleic acid attached thereto;

transferring the nonspecific functionalized support material with nucleic acid attached thereto to a different process chamber through the conduit by rotating the device;
wherein the nucleic acid contacts an amplification reagent located in the different process chamber;
wherein the amplification reagent comprises one or more primers, optionally one or more probes, and one or more amplification enzymes; and
wherein the one or more amplification enzymes are thermally stable at the temperature effective to release and/or denature the nucleic acid;

heating the nonspecific functionalized support material with nucleic acid attached thereto at a temperature effective to release and/or denature the nucleic acid in the presence of the amplification reagent; and

providing conditions effective to amplify the released and/or denatured nucleic acid to produce amplicons.

5. The method of any one of embodiments 1 through 4 wherein the nonspecific functionalized support material contacting the released nucleic acid comprises particulate material.
6 The method of any one of embodiments 1 through 5 wherein the plurality of —C(O)O⁻ or —P(O)(—OH)₂₋ₓ(—O⁻)ₓ groups is a plurality of —C(O)O⁻ groups.
7. The method of any one of embodiments 1 through 6 wherein M is selected from the group consisting of zirconium, gallium, and iron.
8. The method of any one of embodiments 1 through 7 wherein y is 3 or 4.
9. The method of any one of embodiments 1 through 8 wherein M^{y+} is Zr⁴⁺ or Ga³⁺.
10. The method of any one of embodiments 1 through 9 wherein the conditions effective to capture at least a portion of the nucleic acid by the nonspecific functionalized support material comprise a pH of 4.5 to 9.
11. A method of amplifying nucleic acid, the method comprising:

providing a device comprising a process array that comprises process chambers defining volumes for containing sample material or portions thereof, a conduit connecting the process chambers, and a thermal transfer structure comprising a thermal drive chamber (which can be a waste chamber) and resident fluid (gas and/or liquid);

delivering sample material comprising nucleic acid-containing material to a process chamber of the device;

optionally contacting the nucleic acid-containing material with functionalized support material (preferably, nonspecific functionalized support material) under conditions effective to capture at least a portion of the nucleic acid-containing material by the functionalized support material in the process chamber;

optionally transferring the functionalized support material and captured nucleic acid-containing material to a different process chamber;

contacting the nucleic acid-containing material, optionally attached to the functionalized support material, with one or more lysing reagents (which can be included in a lysis buffer) under conditions effective to lyse at least a portion of the nucleic acid-containing material and release nucleic acid;

in the same or a different process chamber, contacting the released nucleic acid with functionalized support material (preferably, nonspecific functionalized support material), which may be the same or different than the functionalized support material optionally used to capture the nucleic acid-containing material, under conditions effective to capture at least a portion of the nucleic acid by the same or different functionalized support material;

washing and/or decanting the functionalized support material with nucleic acid attached thereto using the thermal transfer structure, comprising:
optionally adding a wash solution to the process chamber comprising the functionalized support material with nucleic acid attached thereto;
passing a first portion of the resident fluid (typically, gas) through a thermal transfer conduit into the process chamber comprising the functionalized support material with nucleic acid attached thereto by heating at least a portion of the resident fluid in the thermal drive chamber (typically, a waste chamber), wherein the thermal transfer conduit connects the thermal drive chamber to the process chamber comprising the functionalized support material with nucleic acid attached thereto, and wherein the volume of the resident fluid within the thermal transfer structure increases to force the first portion of the resident fluid into the process chamber comprising the functionalized support material with nucleic acid attached thereto; and
subsequently cooling the heated resident fluid in the thermal transfer structure, wherein the volume of the resident fluid within the thermal transfer structure decreases such that at least a portion of the liquid present in the process chamber comprising the functionalized support material with nucleic acid attached thereto is drawn into the thermal drive chamber through the thermal transfer conduit;

transferring the functionalized support material with nucleic acid attached thereto to a different process chamber through the conduit by rotating the device:

before, during, or after transferring the functionalized support material with nucleic acid attached thereto, contacting it with an amplification reagent, wherein the amplification reagent comprises one or more primers and optionally one or more probes;

optionally heating the functionalized support material with nucleic acid attached thereto at a temperature effective to release and/or denature at least a portion of the captured nucleic acid in the presence of the amplification reagent; and

contacting the nucleic acid, which is optionally released and/or denatured from the functionalized support material, and the amplification reagent with one or more amplification enzymes under conditions effective to amplify the nucleic acid to produce amplicons.

12. A method of amplifying nucleic acid, the method comprising:

providing sample material comprising nucleic acid-containing material;

contacting the nucleic acid-containing material with functionalized support material (preferably, nonspecific functionalized support material) under conditions effective to capture at least a portion of the nucleic acid-containing material by the functionalized support material;

providing a device comprising a process array that comprises process chambers defining volumes for containing sample material or portions thereof, a conduit connecting the process chambers, and a thermal transfer structure comprising a thermal drive chamber (which can be a waste chamber) and resident fluid (gas and/or liquid);

delivering functionalized support material and captured nucleic acid-containing material to a process chamber of the device;

contacting the nucleic acid-containing material with one or more lysing reagents (which can be included in a lysis buffer) in the process chamber under conditions effective to lyse at least a portion of the nucleic acid-containing material and release nucleic acid;

in the same or a different process chamber, contacting the released nucleic acid with functionalized support material (preferably, nonspecific functionalized support material), which may be the same or different than the functionalized support material used to capture the nucleic acid-containing material, under conditions effective to capture at least a portion of the nucleic acid by the same or different functionalized support material;

washing and/or decanting the functionalized support material with nucleic acid attached thereto using the thermal transfer structure, comprising:
optionally adding a wash solution to the process chamber comprising the functionalized support material with nucleic acid attached thereto;
passing a first portion of the resident fluid (typically, gas) through a thermal transfer conduit into the process chamber comprising the functionalized support material with nucleic acid attached thereto by heating at least a portion of the resident fluid in the thermal drive chamber (typically, a waste chamber), wherein the thermal transfer conduit connects the thermal drive chamber to the process chamber comprising the functionalized support material with nucleic acid attached thereto, and wherein the volume of the resident fluid within the thermal drive transfer structure increases to force the first portion of the resident fluid into the process chamber comprising the functionalized support material with nucleic acid attached thereto; and
subsequently cooling the heated resident fluid in the thermal transfer structure, wherein the volume of the resident fluid within the thermal transfer structure decreases such that at least a portion of the liquid present in the process chamber comprising the functionalized support material with nucleic acid attached thereto is drawn into the thermal drive chamber through the thermal transfer conduit;

transferring the functionalized support material with nucleic acid attached thereto to a different process chamber through the conduit by rotating the device:

before, during, or after transferring the functionalized support material with nucleic acid attached thereto, contacting it with an amplification reagent, wherein the amplification reagent comprises one or more primers and optionally one or more probes;

optionally heating the functionalized support material with nucleic acid attached thereto at a temperature effective to release and/or denature at least a portion of the captured nucleic acid in the presence of the amplification reagent; and

contacting the nucleic acid, which is optionally released and/or denatured from the functionalized support material, and the amplification reagent with one or more amplification enzymes under conditions effective to amplify the nucleic acid to produce amplicons.

13. A method of amplifying nucleic acid, the method comprising:

providing a device comprising a process array that comprises process chambers defining volumes for containing sample material or portions thereof, a conduit connecting the process chambers, and a thermal transfer structure comprising a thermal drive chamber (which can be a waste chamber) and resident fluid (gas and/or liquid);

delivering sample material comprising nucleic acid-containing material to a process chamber of the device;

optionally contacting the nucleic acid-containing material with functionalized support material (preferably, nonspecific functionalized support material) under conditions effective to capture at least a portion of the nucleic acid-containing material by the functionalized support material in the process chamber;

optionally transferring the functionalized support material and captured nucleic acid-containing material to a different process chamber;

contacting the nucleic acid-containing material, optionally attached to the functionalized support material, with one or more lysing reagents (which can be included in a lysis buffer) under conditions effective to lyse at least a portion of the nucleic acid-containing material and release nucleic acid;

in the same or a different process chamber, contacting the released nucleic acid with functionalized support material (preferably, nonspecific functionalized support material), which may be the same or different than the functionalized support material optionally used to capture the nucleic acid-containing material, under conditions effective to capture at least a portion of the nucleic acid by the same or different functionalized support material;

washing and/or decanting the functionalized support material with nucleic acid attached thereto using the thermal transfer structure, comprising:
optionally adding a wash solution to the process chamber comprising the functionalized support material with nucleic acid attached thereto;
passing a first portion of the resident fluid (typically, gas) through a thermal transfer conduit into the process chamber comprising the functionalized support material with nucleic acid attached thereto by heating at least a portion of the resident fluid in the thermal drive chamber (typically, a waste chamber), wherein the thermal transfer conduit connects the thermal drive chamber to the process chamber comprising the functionalized support material with nucleic acid attached thereto, and wherein the volume of the resident fluid within the thermal transfer structure increases to force the first portion of the resident fluid into the process chamber comprising the functionalized support material with nucleic acid attached thereto; and
subsequently cooling the heated resident fluid in the thermal transfer structure, wherein the volume of the resident fluid within the thermal transfer structure decreases such that at least a portion of the liquid present in the process chamber comprising the functionalized support material with nucleic acid attached thereto is drawn into the thermal drive chamber through the thermal transfer conduit;

transferring the functionalized support material with nucleic acid attached thereto to a different process chamber through the conduit by: rotating the device;
wherein the nucleic acid contacts an amplification reagent located in the different process chamber;
wherein the amplification reagent comprises one or more primers, optionally one or more probes, and one or more amplification enzymes; and
wherein the one or more amplification enzymes are thermally stable at the temperature effective to release and/or denature the nucleic acid;

heating the functionalized support material with nucleic acid attached thereto at a temperature effective to release and/or denature at least a portion of the nucleic acid in the presence of the amplification reagent; and

providing conditions effective to amplify the released and/or denatured nucleic acid to produce amplicons.

14. A method of amplifying nucleic acid, the method comprising:

providing sample material comprising nucleic acid-containing material;

contacting the nucleic acid-containing material with functionalized support material (preferably, nonspecific functionalized support material) under conditions effective to capture at least a portion of the nucleic acid-containing material by the functionalized support material;

providing a device comprising a process array that comprises process chambers defining volumes for containing sample material or portions thereof, a conduit connecting the process chambers, and a thermal transfer structure comprising a thermal drive chamber (which can be a waste chamber) and resident fluid (gas and/or liquid);

delivering functionalized support material and captured nucleic acid-containing material to a process chamber of the device;

contacting the captured nucleic acid-containing material with one or more lysing reagents (which can be included in a lysis buffer) in the process chamber under conditions effective to lyse at least a portion of the nucleic acid-containing material and release nucleic acid;

in the same or a different process chamber, contacting the released nucleic acid with functionalized support material (preferably, nonspecific functionalized support material), which may be the same or different than the functionalized support material used to capture the nucleic acid-containing material, under conditions effective to capture at least a portion of the nucleic acid by the same or different functionalized support material;

washing and/or decanting the functionalized support material with nucleic acid attached thereto using the thermal transfer structure, comprising:
optionally adding a wash solution to the process chamber comprising the functionalized support material with nucleic acid attached thereto;
passing a first portion of the resident fluid (typically, gas) through a thermal transfer conduit into the process chamber comprising the functionalized support material with nucleic acid attached thereto by heating at least a portion of the resident fluid in the thermal drive chamber (typically, a waste chamber), wherein the thermal transfer conduit connects the thermal drive chamber to the process chamber comprising the functionalized support material with nucleic acid attached thereto, and wherein the volume of the resident fluid within the thermal transfer structure increases to force the first portion of the resident fluid into the process chamber comprising the functionalized support material with nucleic acid attached thereto; and
subsequently cooling the heated resident fluid in the thermal transfer structure, wherein the volume of the resident fluid within the thermal transfer structure decreases such that at least a portion of the liquid present in the process chamber comprising the functionalized support material with nucleic acid attached thereto is drawn into the thermal drive chamber through the thermal transfer conduit;

transferring the functionalized support material with nucleic acid attached thereto to a different process chamber through the conduit by rotating the device;
wherein the nucleic acid contacts an amplification reagent located in the different process chamber;
wherein the amplification reagent comprises one or more primers, optionally one or more probes, and one or more amplification enzymes; and
wherein the one or more amplification enzymes are thermally stable at the temperature effective to release and/or denature the nucleic acid;

heating the functionalized support material with nucleic acid attached thereto at a temperature effective to release and/or denature the nucleic acid in the presence of the amplification reagent; and

providing conditions effective to amplify the released and/or denatured nucleic acid to produce amplicons.

15. A method of amplifying nucleic acid, the method comprising:

providing a device comprising a process array that comprises process chambers defining volumes for containing sample material or portions thereof and a conduit connecting the process chambers;

delivering sample material comprising nucleic acid-containing material to a process chamber of the device;

optionally contacting the nucleic acid-containing material with functionalized support material (preferably, nonspecific functionalized support material) under conditions effective to capture at least a portion of the nucleic acid-containing material by the functionalized support material in the process chamber;

optionally transferring the functionalized support material and captured nucleic acid-containing material to a different process chamber;

contacting the nucleic acid-containing material, optionally attached to the functionalized support material, with one or more lysing reagents (which can be included in a lysis buffer) under conditions effective to lyse at least a portion of the nucleic acid-containing material and release nucleic acid;

in the same or a different process chamber, contacting the released nucleic acid with functionalized support material (preferably, nonspecific functionalized support material), which may be the same or different than the functionalized support material optionally used to capture the nucleic acid-containing material, under conditions effective to capture at least a portion of the nucleic acid by the same or different functionalized support material;

washing the functionalized support material with nucleic acid attached thereto multiple times in the same process chamber using multiple aliquots of one or more wash solutions;

transferring the functionalized support material with nucleic acid attached thereto to a different process chamber through the conduit by rotating the device;

before, during, or after transferring the functionalized support material with nucleic acid attached thereto, contacting it with an amplification reagent, wherein the amplification reagent comprises one or more primers and optionally one or more probes;

optionally heating the functionalized support material with nucleic acid attached thereto at a temperature effective to release and/or denature at least a portion of the captured nucleic acid in the presence of the amplification reagent; and

contacting the nucleic acid, which is optionally released and/or denatured from the functionalized support material, and the amplification reagent with one or more amplification enzymes under conditions effective to amplify the nucleic acid to produce amplicons.

16. A method of amplifying nucleic acid, the method comprising:

providing sample material comprising nucleic acid-containing material;

contacting the nucleic acid-containing material with functionalized support material (preferably, nonspecific functionalized support material) under conditions effective to capture at least a portion of the nucleic acid-containing material by the functionalized support material;

providing a device comprising a process array that comprises process chambers defining volumes for containing sample material or portions thereof and a conduit connecting the process chambers;

delivering functionalized support material and captured nucleic acid-containing material to a process chamber of the device;

contacting the nucleic acid-containing material with one or more lysing reagents (which can be included in a lysis buffer) in the process chamber under conditions effective to lyse at least a portion of the nucleic acid-containing material and release nucleic acid;

in the same or a different process chamber, contacting the released nucleic acid with functionalized support material (preferably, nonspecific functionalized support material), which may be the same or different than the functionalized support material used to capture the nucleic acid-containing material, under conditions effective to capture at least a portion of the nucleic acid by the same or different functionalized support material;

washing the functionalized support material with nucleic acid attached thereto multiple times in the same process chamber using multiple aliquots of one or more wash solutions;

transferring the functionalized support material with nucleic acid attached thereto to a different process chamber through the conduit by rotating the device;

before, during, or after transferring the functionalized support material with nucleic acid attached thereto, contacting it with an amplification reagent, wherein the amplification reagent comprises one or more primers and optionally one or more probes;

optionally heating the functionalized support material with nucleic acid attached thereto at a temperature effective to release and/or denature at least a portion of the captured nucleic acid in the presence of the amplification reagent; and

contacting the nucleic acid, which is optionally released and/or denatured from the functionalized support material, and the amplification reagent with one or more amplification enzymes under conditions effective to amplify the nucleic acid to produce amplicons.

17. A method of amplifying nucleic acid, the method comprising:

providing a device comprising a process array that comprises process chambers defining volumes for containing sample material or portions thereof and a conduit connecting the process chambers;

delivering sample material comprising nucleic acid-containing material to a process chamber of the device;

optionally contacting the nucleic acid-containing material with functionalized support material (preferably, nonspecific functionalized support material) under conditions effective to capture at least a portion of the nucleic acid-containing material by the functionalized support material in the process chamber;

optionally transferring the functionalized support material and captured nucleic acid-containing material to a different process chamber;

contacting the nucleic acid-containing material, optionally attached to the functionalized support material, with one or more lysing reagents (which can be included in a lysis buffer) under conditions effective to lyse at least a portion of the nucleic acid-containing material and release nucleic acid;

in the same or a different process chamber, contacting the released nucleic acid with functionalized support material (preferably, nonspecific functionalized support material), which may be the same or different than the functionalized support material optionally used to capture the nucleic acid-containing material, under conditions effective to capture at least a portion of the nucleic acid by the same or different functionalized support material;

washing the functionalized support material with nucleic acid attached thereto multiple times in the same process chamber using multiple aliquots of one or more wash solutions;

transferring the functionalized support material with nucleic acid attached thereto to a different process chamber through the conduit by rotating the device;
wherein the nucleic acid contacts an amplification reagent located in the different process chamber;
wherein the amplification reagent comprises one or more primers, optionally one or more probes, and one or more amplification enzymes; and
wherein the one or more amplification enzymes are thermally stable at the temperature effective to release and/or denature the nucleic acid;

heating the functionalized support material with nucleic acid attached thereto at a temperature effective to release and/or denature at least a portion of the nucleic acid in the presence of the amplification reagent; and

providing conditions effective to amplify the released and/or denatured nucleic acid to produce amplicons.

18. A method of amplifying nucleic acid, the method comprising:

providing sample material comprising nucleic acid-containing material;

contacting the nucleic acid-containing material with functionalized support material (preferably, nonspecific functionalized support material) under conditions effective to capture at least a portion of the nucleic acid-containing material by the functionalized support material;

providing a device comprising a process array that comprises process chambers defining volumes for containing sample material or portions thereof and a conduit connecting the process chambers;

delivering functionalized support material and captured nucleic acid-containing material to a process chamber of the device;

contacting the captured nucleic acid-containing material with one or more lysing reagents (which can be included in a lysis buffer) in the process chamber under conditions effective to lyse at least a portion of the nucleic acid-containing material and release nucleic acid;

in the same or a different process chamber, contacting the released nucleic acid with functionalized support material (preferably, nonspecific functionalized support material), which may be the same or different than the functionalized support material used to capture the nucleic acid-containing material, under conditions effective to capture at least a portion of the nucleic acid by the same or different functionalized support material;

washing the functionalized support material with nucleic acid attached thereto multiple times in the same process chamber using multiple aliquots of one or more wash solutions;

transferring the functionalized support material with nucleic acid attached thereto to a different process chamber through the conduit by rotating the device;
wherein the nucleic acid contacts an amplification reagent located in the different process chamber;
wherein the amplification reagent comprises one or more primers, optionally one or more probes, and one or more amplification enzymes; and
wherein the one or more amplification enzymes are thermally stable at the temperature effective to release and/or denature the nucleic acid;

heating the functionalized support material with nucleic acid attached thereto at a temperature effective to release and/or denature the nucleic acid in the presence of the amplification reagent; and

providing conditions effective to amplify the released and/or denatured nucleic acid to produce amplicons.

19. The method of any one of embodiments 11 through 18 wherein the functionalized support material contacting the released nucleic acid comprises particulate material.
20. The method of any one of embodiments 11 through 19 wherein the functionalized support material is nonspecific functionalized support material that comprises immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; wherein M is selected from the group consisting of zirconium, gallium, iron, aluminum, scandium, titanium, vanadium, yttrium, and a lanthanide; y is an integer from 3 to 6; and x is 1 or 2.
21. The method of embodiment 20 wherein the plurality of —C(O)O⁻ or —P(O)(—OH)₂₋ₓ(—O⁻)ₓ groups is a plurality of —C(O)O⁻ groups.
22. The method of embodiment 20 or embodiment 21, wherein M is selected from the group consisting of zirconium, gallium, and iron.
23. The method of any one of embodiments 20 through 22 wherein y is 3 or 4.
24. The method of any one of embodiments 21 through 23 wherein M^{y+} is Zr⁴⁺ or Ga³⁺.
25. The method of any one of embodiments 11 through 24 wherein the conditions effective to capture at least a portion of the nucleic acid by the nonspecific functionalized support material comprise a pH of 4.5 to 9.
26. The method of any one of embodiments 1 through 25 wherein washing the functionalized support material with nucleic acid attached thereto comprises:

introducing a wash buffer into the process chamber and mixing to form a mixture;

optionally compacting the functionalized support material (preferably, nonspecific functionalized support material) with nucleic acid attached thereto after mixing;

removing a majority of the components of the mixture leaving the functionalized support material with nucleic acid attached thereto in the process chamber; and

optionally repeating the introducing and removing steps one or more times.

27. The method of embodiment 1, embodiment 3, embodiment 11, embodiment 13, embodiment 15, embodiment 17, and any one of embodiments 5 through 10 and 19 through 25 as dependent on embodiment 1, embodiment 3, embodiment 11, embodiment 13, embodiment 15, or embodiment 17, wherein the optional steps of contacting and transferring the functionalized support material and captured nucleic acid-containing material are included in the process.
28. The method of embodiment 2, embodiment 4, embodiment 12, embodiment 14, embodiment 16, embodiment 18, and any one of embodiments 5 through 10 and 19 through 26 as dependent on embodiment 2, embodiment 4, embodiment 12, embodiment 14, embodiment 16, or embodiment 18, wherein the conditions effective to capture at least a portion of the nucleic acid-containing material by the functionalized support material (preferably, nonspecific functionalized support material) comprise a pH of 4.5 to 9.
29. The method of any one of embodiments 1 through 28 further comprising washing the functionalized support material (preferably, nonspecific functionalized support material) and captured nucleic acid-containing material.
30 The method of any one of embodiments 1 through 29 wherein contacting the nucleic acid-containing material with one or more lysing reagents (which can be included in a lysis buffer) and contacting the released nucleic acid with functionalized support material (preferably, nonspecific functionalized support material) occur in the same chamber.
31. The method of embodiment 30 wherein the one or more lysing reagents (which can be included in a lysis buffer) comprises an enzymatic lysing reagent.
32. The method of embodiment 30 wherein the one or more lysing reagents (which can be included in a lysis buffer) comprises a basic lysing reagent.
33. The method of embodiment 32 wherein the one or more lysing reagents (which can be included in a lysis buffer) further comprises a neutralization reagent.
34. The method of embodiment 32 further comprising contacting the functionalized support material (preferably, nonspecific functionalized support material) having nucleic acid attached thereto with a neutralization reagent.
35. The method of any one of embodiments 1 through 29 wherein contacting the nucleic acid-containing material with one or more lysing reagents (which can be included in a lysis buffer) and contacting the released nucleic acid with functionalized support material (preferably, nonspecific functionalized support material) occur in the same or different chambers.

36. The method of embodiment 35 wherein the one or more lysing reagents (which can be included in a lysis buffer) comprises an enzymatic lysing reagent.

37. The method of embodiment 35 wherein the one or more lysing reagents (which can be included in a lysis buffer) comprises a basic lysing reagent.
38. The method of embodiment 37 wherein the process chamber in which the functionalized support material (preferably, nonspecific functionalized support material) is located includes a neutralization reagent.
39. The method of embodiment 37 wherein prior to contacting the released nucleic acid with functionalized support material, the method includes contacting the released nucleic acid with a neutralization reagent.
40. The method of embodiment 1, embodiment 3, embodiment 11, embodiment 13, embodiment 15, embodiment 17, and any one of embodiments 5 through 10, 19 through 27, and 29 through 39 as dependent on embodiment 1, embodiment 3, embodiment 11, embodiment 13, embodiment 15, or embodiment 17, wherein the nucleic acid-containing material comprises bacterial cells, and the method comprises:

delivering sample material comprising bacterial cells to a process chamber;

contacting the bacterial cells with functionalized support material (preferably, nonspecific functionalized support material) under conditions effective to capture at least a portion of the bacterial cells by the functionalized support material in the process chamber;

transferring the functionalized support material and captured bacterial cells to a different process chamber; and

contacting the bacterial cells attached to the functionalized support material with one or more lysing reagents (which can be included in a lysis buffer) comprising an enzyme under conditions effective to lyse at least a portion of the bacterial cells, release nucleic acid, and capture at least a portion of the nucleic acid by the same functionalized support material (preferably, nonspecific functionalized support material) to which the bacterial cells were attached.

41. The method of embodiment 1, embodiment 3, embodiment 11, embodiment 13, embodiment 15, embodiment 17, and any one of embodiments 5 through 10, 19 through 27, and 29 through 39 as dependent on embodiment 1, embodiment 3, embodiment 11, embodiment 13, embodiment 15, or embodiment 17, wherein the nucleic acid-containing material comprises bacterial cells, and the method comprises:

delivering sample material comprising bacterial cells to a process chamber;

contacting the bacterial cells with functionalized support material (preferably, nonspecific functionalized support material) under conditions effective to capture at least a portion of the bacterial cells by the functionalized support material in the process chamber;

transferring the functionalized support material and captured bacterial cells to a different process chamber; and

contacting the bacterial cells attached to the functionalized support material with one or more lysing reagents (which can be included in a lysis buffer) comprising a base under conditions effective to lyse at least a portion of the bacterial cells, release nucleic acid, and capture at least a portion of the nucleic acid by the same functionalized support material (preferably, nonspecific functionalized support material) to which the bacterial cells were attached.

42. The method of embodiment 41 further comprising contacting the released nucleic acid with a neutralization reagent to increase the amount of nucleic acid attached to the functionalized support material (preferably, nonspecific functionalized support material).
43. The method of embodiment 42 wherein the one or more lysing reagents (which can be included in a lysis buffer) and the neutralization reagent are dried and spatially separated within the same chamber prior to contact with the functionalized support material and captured bacterial cells.
44. The method of embodiment 42 wherein the neutralization reagent and one or more lysing reagents (which can be included in a lysis buffer) are in different chambers.
45. The method of embodiment 1, embodiment 3, embodiment 11, embodiment 13, embodiment 15, embodiment 17, and any one of embodiments 5 through 10, 19 through 27, 29, and 35 through 39 as dependent on embodiment 1, embodiment 3, embodiment 11, embodiment 13, embodiment 15, or embodiment 17, wherein the nucleic acid-containing material comprises bacteria, and the method comprises:

delivering sample material comprising bacterial cells to a process chamber;

contacting the bacterial cells with functionalized support material (preferably, nonspecific functionalized support material) under conditions effective to capture at least a portion of the bacterial cells by the functionalized support material in the process chamber;

transferring the functionalized support material and captured bacterial cells to a different process chamber; and

contacting the bacterial cells attached to the functionalized support material, with one or more lysing reagents (which can be included in a lysis buffer) under conditions effective to lyse at least a portion of the bacterial cells and release nucleic acid;

in a different process chamber, contacting the released nucleic acid with functionalized support material (preferably, nonspecific functionalized support material), which may be the same or different than the functionalized support material optionally used to capture the bacterial cells, under conditions effective to capture at least a portion of the nucleic acid by the same or different functionalized support material.

46. The method of embodiment 45 wherein at least a portion of the released nucleic acid is captured by different functionalized support material than used to capture the bacterial cells.
47. The method of any one of embodiments 1 through 46 wherein the lysing reagent (which can be included in a lysis buffer) comprises an enzyme, a base, a surfactant, or combinations thereof
48. The method of embodiment 47 wherein the lysing reagent (which can be included in a lysis buffer) comprises an enzyme and a surfactant.
49. The method of embodiment 47 wherein the lysing reagent is in a lysis buffer that comprises a protease lysing reagent, a surfactant, and a chelator.
50. The method of embodiment 47 wherein the lysing reagent is in a lysis buffer that comprises a cell wall peptidoglycan degrading enzyme, a surfactant, a chelator, and a RNase or DNase inhibitor.
51. The method of embodiment 47 wherein the lysing reagent is in a lysis buffer that comprises a base and a surfactant.
52. The method of embodiment 47 wherein the lysing reagent is in a lysis buffer that comprises a basic lysing reagent and the method further includes contacting the released nucleic acid, optionally attached to functionalized support material, with a neutralization reagent.
53. The method of embodiment 52 wherein the neutralization reagent comprises an inorganic acid solution or an acidic buffer.
54. The method of any one of embodiments 1 through 53 wherein the lysing reagent is in a lysis buffer that further comprises a protein denaturant.
55. The method of any one of embodiments 1 through 53 wherein the lysing reagent is in a lysis buffer that further comprises guanidine isothiocyanate, guanidine hydrochloride, urea, or combinations thereof.
56. The method of any one of embodiments 1 through 55 wherein the sample material further comprises a surfactant.
57. The method of any one of embodiments 1 through 56 wherein the sample material includes a plurality of cells, viruses, or a combination thereof
58. The method of embodiment 57 wherein the sample material includes a plurality of cells.
59. The method of embodiment 58 wherein the cells are bacterial cells.
60. The method of embodiment 1, embodiment 2, embodiment 11, embodiment 12, embodiment 15, embodiment 16, and any one of embodiments 5 through 10 and embodiments 19 through 59 as dependent on embodiment 1, embodiment 2, embodiment 11, embodiment 12, embodiment 15, or embodiment 16, wherein the optional step of heating the functionalized support material with nucleic acid attached thereto is included in the process.
61. The method of any one of embodiments 1 through 60 wherein when the functionalized support material with nucleic acid attached thereto is heated, the method comprises heating at a temperature of 50° C. to 99° C.
62. The method of any one of embodiments 1 through 61 wherein prior to heating the functionalized support material with nucleic acid attached thereto, the method further comprises compacting the functionalized support material with nucleic acid attached thereto.
63. The method of any one of embodiments 1 through 61 wherein after heating the functionalized support material with nucleic acid attached thereto to denature and/or release the nucleic acid, the method further comprises compacting the functionalized support material.
64. The method of any one of embodiments 1 through 63 comprising a mixture of amplification enzymes.
65. The method of any one of embodiments 1 through 64 further comprising detecting the amplified nucleic acid.
66. The method of any one of embodiments 1 through 65 wherein contacting the nucleic acid-containing material with one or more lysing reagents (which can be included in a lysis buffer) comprises adding the one or more lysing reagents (which can be included in a lysis buffer) to the process chamber after the nucleic acid-containing material is in the process chamber.
67. The method of embodiment 1, embodiment 2, embodiment 11, embodiment 12, embodiment 15, embodiment 16, and any one of embodiments 5 through 10 and embodiments 19 through 66 as dependent on embodiment 1, embodiment 2, embodiment 11, embodiment 12, embodiment 15, or embodiment 16, wherein prior to contacting the nucleic acid and the amplification reagent with one or more amplification enzymes, the method comprises transferring the nucleic acid to a different process chamber containing the one or more amplification enzymes.
68. The method of any one of embodiments 1 through 67 wherein prior to carrying out the method on the device, the one or more lysing reagents (which can be included in a lysis buffer), amplification reagent, and/or functionalized support material if present are in dry form in one or more process chambers.
69. The method of embodiment 1, embodiment 3, embodiment 11, embodiment 13, embodiment 15, embodiment 17, and any one of embodiments 5 through 10, embodiments 19 through 27, and embodiments 29 through 68 as dependent on embodiment 1, embodiment 3, embodiment 11, embodiment 13, embodiment 15, embodiment 17, wherein the process chamber to which the sample material is delivered contains the functionalized support material in dry form prior to delivery of the sample material.
70. The method of any one of embodiments 1 through 69 wherein heating the functionalized support material with nucleic acid attached thereto comprises heating at a temperature effective to denature and release at least a portion of the nucleic acid from the functionalized support material.
71. The method of any one of embodiments 1 through 70 wherein the process chamber to which the sample material is delivered contains an internal control.
72. The method of any one of embodiments 1 through 70 wherein the sample material includes an internal control.
73. The method of embodiment 71 or embodiment 72 wherein the internal control comprises nucleic acid and/or cells from a known microorganism.
74. The method of any one of embodiments 1 through 70 wherein the sample material further includes an enrichment broth comprising a microorganism of interest.
75. The method of any one of embodiments 1 through 74 wherein the functionalized support material (preferably, nonspecific functionalized support material) is functionalized to capture bacteria, fungi, or both.
76. The method of embodiment 75 wherein the functionalized support material (preferably, nonspecific functionalized support material) is functionalized to capture gram negative bacteria, gram positive bacteria, or both.
77. The method of embodiment 76 wherein the lysing reagent (which can be included in a lysis buffer) comprises a mixture of lysing reagents.
78, The method of embodiment 77 wherein the mixture of lysing reagents (which can be included in a lysis buffer) comprises a lysing reagent for gram negative bacteria and a lysing reagent for gram positive bacteria.
79. The method of any one of embodiments 1 through 78 further comprising transferring the amplicons to a multi-array detection assay.
80. The method of any one of embodiments 1 through 78 wherein the amplification occurs in a multi-array amplification system.
81. The method of any one of embodiments 1 through 80 wherein the one or more amplification probes are present.
82. The method of any one of embodiments 1 through 81 further comprising transferring the amplicons to a different chamber comprising an amplification reagent for a second type of amplification reaction, wherein the amplification reagent comprises one or more primers, one or more probes, and one or more enzymes for the second type of amplification reaction.
83. A method of amplifying nucleic acid, the method comprising:

providing a device comprising a process array that comprises process chambers defining volumes for containing sample material or portions thereof and a conduit connecting the process chambers;

delivering sample material comprising lysed nucleic acid-containing material that includes nucleic acid to a process chamber of the device;

contacting the nucleic acid with nonspecific functionalized support material under conditions effective to capture at least a portion of the nucleic acid, wherein the nonspecific functionalized support material contacting the released nucleic acid comprises immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; wherein M is selected from the group consisting of zirconium, gallium, iron, aluminum, scandium, titanium, vanadium, yttrium, and a lanthanide; y is an integer from 3 to 6; and x is 1 or 2;

washing the nonspecific functionalized support material with nucleic acid attached thereto;

transferring the nonspecific functionalized support material with nucleic acid attached thereto to a different process chamber through the conduit by rotating the device;

before, during, or after transferring the nonspecific functionalized support material with nucleic acid attached thereto, contacting it with an amplification reagent, wherein the amplification reagent comprises one or more primers and optionally one or more probes;

optionally heating the nonspecific functionalized support material with nucleic acid attached thereto at a temperature effective to release and/or denature at least a portion of the captured nucleic acid in the presence of the amplification reagent; and

contacting the nucleic acid, which is optionally released and/or denatured from the nonspecific functionalized support material, and the amplification reagent with one or more amplification enzymes under conditions effective to amplify the nucleic acid to produce amplicons.

84. A method of amplifying nucleic acid, the method comprising:

providing sample material comprising lysed nucleic acid-containing material that includes nucleic acid;

contacting the nucleic acid-containing material with nonspecific functionalized support material under conditions effective to capture at least a portion of the nucleic acid by the nonspecific functionalized support material, wherein the nonspecific functionalized support material contacting the released nucleic acid comprises immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; wherein M is selected from the group consisting of zirconium, gallium, iron, aluminum, scandium, titanium, vanadium, yttrium, and a lanthanide; y is an integer from 3 to 6; and x is 1 or 2;

providing a device comprising a process array that comprises process chambers defining volumes for containing sample material or portions thereof and a conduit connecting the process chambers;

delivering nonspecific functionalized support material and captured nucleic acid to a process chamber of the device;

washing the nonspecific functionalized support material with nucleic acid attached thereto;

transferring the nonspecific functionalized support material with nucleic acid attached thereto to a different process chamber through the conduit by rotating the device;

before, during, or after transferring the nonspecific functionalized support material with nucleic acid attached thereto, contacting it with an amplification reagent, wherein the amplification reagent comprises one or more primers and optionally one or more probes;

optionally heating the nonspecific functionalized support material with nucleic acid attached thereto at a temperature effective to release and/or denature at least a portion of the captured nucleic acid in the presence of the amplification reagent; and

contacting the nucleic acid, which is optionally released and/or denatured from the nonspecific functionalized support material, and the amplification reagent with one or more amplification enzymes under conditions effective to amplify the nucleic acid to produce amplicons.

85. A method of amplifying nucleic acid, the method comprising:

providing a device comprising a process array that comprises process chambers defining volumes for containing sample material or portions thereof and a conduit connecting the process chambers;

delivering sample material comprising lysed nucleic acid-containing material that includes nucleic acid to a process chamber of the device;

contacting the nucleic acid with nonspecific functionalized support material under conditions effective to capture at least a portion of the nucleic acid, wherein the nonspecific functionalized support material contacting the released nucleic acid comprises immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; wherein M is selected from the group consisting of zirconium, gallium, iron, aluminum, scandium, titanium, vanadium, yttrium, and a lanthanide; y is an integer from 3 to 6; and x is 1 or 2;

washing the nonspecific functionalized support material with nucleic acid attached thereto;

transferring the nonspecific functionalized support material with nucleic acid attached thereto to a different process chamber through the conduit by rotating the device;
wherein the nucleic acid contacts an amplification reagent located in the different process chamber;
wherein the amplification reagent comprises one or more primers, optionally one or more probes, and one or more amplification enzymes; and
wherein the one or more amplification enzymes are thermally stable at the temperature effective to release and/or denature the nucleic acid;

heating the nonspecific functionalized support material with nucleic acid attached thereto at a temperature effective to release and/or denature at least a portion of the nucleic acid in the presence of the amplification reagent; and

providing conditions effective to amplify the released and/or denatured nucleic acid to produce amplicons.

86. A method of amplifying nucleic acid, the method comprising:

providing sample material comprising lysed nucleic acid-containing material that includes nucleic acid;

contacting the nucleic acid-containing material with nonspecific functionalized support material under conditions effective to capture at least a portion of the nucleic acid by the nonspecific functionalized support material, wherein the nonspecific functionalized support material contacting the released nucleic acid comprises immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; wherein M is selected from the group consisting of zirconium, gallium, iron, aluminum, scandium, titanium, vanadium, yttrium, and a lanthanide; y is an integer from 3 to 6; and x is 1 or 2;

providing a device comprising a process array that comprises process chambers defining volumes for containing sample material or portions thereof and a conduit connecting the process chambers;

delivering nonspecific functionalized support material and captured nucleic acid to a process chamber of the device;

washing the nonspecific functionalized support material with nucleic acid attached thereto;

transferring the nonspecific functionalized support material with nucleic acid attached thereto to a different process chamber through the conduit by rotating the device;
wherein the nucleic acid contacts an amplification reagent located in the different process chamber;
wherein the amplification reagent comprises one or more primers, optionally one or more probes, and one or more amplification enzymes; and
wherein the one or more amplification enzymes are thermally stable at the temperature effective to release and/or denature the nucleic acid;

heating the nonspecific functionalized support material with nucleic acid attached thereto at a temperature effective to release and/or denature the nucleic acid in the presence of the amplification reagent; and

providing conditions effective to amplify the released and/or denatured nucleic acid to produce amplicons.

87. A method of amplifying nucleic acid, the method comprising:

providing a device comprising a process array that comprises process chambers defining volumes for containing sample material or portions thereof, a conduit connecting the process chambers, and a thermal transfer structure comprising a thermal drive chamber (which can be a waste chamber) and resident fluid (gas and/or liquid);

delivering sample material comprising lysed nucleic acid-containing material that includes nucleic acid to a process chamber of the device;

contacting the nucleic acid with functionalized support material (preferably, nonspecific functionalized support material) under conditions effective to capture at least a portion of the nucleic acid;

washing and/or decanting the functionalized support material with nucleic acid attached thereto using the thermal transfer structure, comprising:
optionally adding a wash solution to the process chamber comprising the functionalized support material with nucleic acid attached thereto;
passing a first portion of the resident fluid (typically, gas) through a thermal transfer conduit into the process chamber comprising the functionalized support material with nucleic acid attached thereto by heating at least a portion of the resident fluid in the thermal drive chamber (typically, a waste chamber), wherein the thermal transfer conduit connects the thermal drive chamber to the process chamber comprising the functionalized support material with nucleic acid attached thereto, and wherein the volume of the resident fluid within the thermal transfer structure increases to force the first portion of the resident fluid into the process chamber comprising the functionalized support material with nucleic acid attached thereto; and
subsequently cooling the heated resident fluid in the thermal transfer structure, wherein the volume of the resident fluid within the thermal transfer structure decreases such that at least a portion of the liquid present in the process chamber comprising the functionalized support material with nucleic acid attached thereto is drawn into the thermal drive chamber through the thermal transfer conduit;

transferring the functionalized support material with nucleic acid attached thereto to a different process chamber through the conduit by rotating the device:

before, during, or after transferring the functionalized support material with nucleic acid attached thereto, contacting it with an amplification reagent, wherein the amplification reagent comprises one or more primers and optionally one or more probes;

optionally heating the functionalized support material with nucleic acid attached thereto at a temperature effective to release and/or denature at least a portion of the captured nucleic acid in the presence of the amplification reagent; and

contacting the nucleic acid, which is optionally released and/or denatured from the functionalized support material, and the amplification reagent with one or more amplification enzymes under conditions effective to amplify the nucleic acid to produce amplicons.

88. A method of amplifying nucleic acid, the method comprising:

providing sample material comprising lysed nucleic acid-containing material that includes nucleic acid;

contacting the nucleic acid-containing material with functionalized support material (preferably, nonspecific functionalized support material) under conditions effective to capture at least a portion of the nucleic acid by the functionalized support material;

providing a device comprising a process array that comprises process chambers defining volumes for containing sample material or portions thereof, a conduit connecting the process chambers, and a thermal transfer structure comprising a thermal drive chamber (which can be a waste chamber) and resident fluid (gas and/or liquid);

delivering functionalized support material and captured nucleic acid to a process chamber of the device;

washing and/or decanting the functionalized support material with nucleic acid attached thereto using the thermal transfer structure, comprising:
optionally adding a wash solution to the process chamber comprising the functionalized support material with nucleic acid attached thereto;
passing a first portion of the resident fluid (typically, gas) through a thermal transfer conduit into the process chamber comprising the functionalized support material with nucleic acid attached thereto by heating at least a portion of the resident fluid in the thermal drive chamber (typically, a waste chamber), wherein the thermal transfer conduit connects the thermal drive chamber to the process chamber comprising the functionalized support material with nucleic acid attached thereto, and wherein the volume of the resident fluid within the thermal transfer structure increases to force the first portion of the resident fluid into the process chamber comprising the functionalized support material with nucleic acid attached thereto; and
subsequently cooling the heated resident fluid in the thermal transfer structure, wherein the volume of the resident fluid within the thermal transfer structure decreases such that at least a portion of the liquid present in the process chamber comprising the functionalized support material with nucleic acid attached thereto is drawn into the thermal drive chamber through the thermal transfer conduit;

transferring the functionalized support material with nucleic acid attached thereto to a different process chamber through conduit by rotating the device:

before, during, or after transferring the functionalized support material with nucleic acid attached thereto, contacting it with an amplification reagent, wherein the amplification reagent comprises one or more primers and optionally one or more probes;

optionally heating the functionalized support material with nucleic acid attached thereto at a temperature effective to release and/or denature at least a portion of the captured nucleic acid in the presence of the amplification reagent; and

contacting the nucleic acid, which is optionally released and/or denatured from the functionalized support material, and the amplification reagent with one or more amplification enzymes under conditions effective to amplify the nucleic acid to produce amplicons.

89. A method of amplifying nucleic acid, the method comprising:

providing a device comprising a process array that comprises process chambers defining volumes for containing sample material or portions thereof, a conduit connecting the process chambers, and a thermal transfer structure comprising a thermal drive chamber (which can be a waste chamber) and resident fluid (gas and/or liquid);

delivering sample material comprising lysed nucleic acid-containing material that includes nucleic acid to a process chamber of the device;

contacting the nucleic acid with functionalized support material (preferably, nonspecific functionalized support material) under conditions effective to capture at least a portion of the nucleic acid;

washing and/or decanting the functionalized support material with nucleic acid attached thereto using the thermal transfer structure, comprising:
optionally adding a wash solution to the process chamber comprising the functionalized support material with nucleic acid attached thereto;
passing a first portion of the resident fluid (typically, gas) through a thermal transfer conduit into the process chamber comprising the functionalized support material with nucleic acid attached thereto by heating at least a portion of the resident fluid in the thermal drive chamber (typically, a waste chamber), wherein the thermal transfer conduit connects the thermal drive chamber to the process chamber comprising the functionalized support material with nucleic acid attached thereto, and wherein the volume of the resident fluid within the thermal transfer structure increases to force the first portion of the resident fluid into the process chamber comprising the functionalized support material with nucleic acid attached thereto; and
subsequently cooling the heated resident fluid in the thermal transfer structure, wherein the volume of the resident fluid within the thermal transfer structure decreases such that at least a portion of the liquid present in the process chamber comprising the functionalized support material with nucleic acid attached thereto is drawn into the thermal drive chamber through the thermal transfer conduit;

transferring the functionalized support material with nucleic acid attached thereto to a different process chamber by rotating the device:
wherein the nucleic acid contacts an amplification reagent located in the different process chamber;
wherein the amplification reagent comprises one or more primers, optionally one or more probes, and one or more amplification enzymes; and
wherein the one or more amplification enzymes are thermally stable at the temperature effective to release and/or denature the nucleic acid;

heating the functionalized support material with nucleic acid attached thereto at a temperature effective to release and/or denature at least a portion of the nucleic acid in the presence of the amplification reagent; and

providing conditions effective to amplify the released and/or denatured nucleic acid to produce amplicons.

90. A method of amplifying nucleic acid, the method comprising:

providing sample material comprising lysed nucleic acid-containing material that includes nucleic acid;

contacting the nucleic acid-containing material with functionalized support material (preferably, nonspecific functionalized support material) under conditions effective to capture at least a portion of the nucleic acid by the functionalized support material;

providing a device comprising a process array that comprises process chambers defining volumes for containing sample material or portions thereof, a conduit connecting the process chambers, and a thermal transfer structure comprising a thermal drive chamber (which can be a waste chamber) and resident fluid (gas and/or liquid);

delivering functionalized support material and captured nucleic acid to a process chamber of the device;

washing and/or decanting the functionalized support material with nucleic acid attached thereto using the thermal transfer structure, comprising:
optionally adding a wash solution to the process chamber comprising the functionalized support material with nucleic acid attached thereto;
passing a first portion of the resident fluid (typically, gas) through a thermal transfer conduit into the process chamber comprising the functionalized support material with nucleic acid attached thereto by heating at least a portion of the resident fluid in the thermal drive chamber (typically, a waste chamber), wherein the thermal transfer conduit connects the thermal drive chamber to the process chamber comprising the functionalized support material with nucleic acid attached thereto, and wherein the volume of the resident fluid within the thermal transfer structure increases to force the first portion of the resident fluid into the process chamber comprising the functionalized support material with nucleic acid attached thereto; and
subsequently cooling the heated resident fluid in the thermal transfer structure, wherein the volume of the resident fluid within the thermal transfer structure decreases such that at least a portion of the liquid present in the process chamber comprising the functionalized support material with nucleic acid attached thereto is drawn into the thermal drive chamber through the thermal transfer conduit;

transferring the functionalized support material with nucleic acid attached thereto to a different process chamber through the conduit by rotating the device:
wherein the nucleic acid contacts an amplification reagent located in the different process chamber;
wherein the amplification reagent comprises one or more primers, optionally one or more probes, and one or more amplification enzymes; and
wherein the one or more amplification enzymes are thermally stable at the temperature effective to release and/or denature the nucleic acid;

heating the functionalized support material with nucleic acid attached thereto at a temperature effective to release and/or denature the nucleic acid in the presence of the amplification reagent; and

providing conditions effective to amplify the released and/or denatured nucleic acid to produce amplicons.

91. A method of amplifying nucleic acid, the method comprising:

providing a device comprising a process array that comprises process chambers defining volumes for containing sample material or portions thereof and a conduit connecting the process chambers;

delivering sample material comprising lysed nucleic acid-containing material that includes nucleic acid to a process chamber of the device;

contacting the nucleic acid with functionalized support material under conditions effective to capture at least a portion of the nucleic acid;

washing the functionalized support material with nucleic acid attached thereto multiple times in the same process chamber using multiple aliquots of one or more wash solutions;

transferring the functionalized support material with nucleic acid attached thereto to a different process chamber through the conduit by rotating the device;

before, during, or after transferring, the functionalized support material with nucleic acid attached thereto, contacting it with an amplification reagent, wherein the amplification reagent comprises one or more primers and optionally one or more probes;

optionally heating the functionalized support material with nucleic acid attached thereto at a temperature effective to release and/or denature at least a portion of the captured nucleic acid in the presence of the amplification reagent; and

contacting the nucleic acid, which is optionally released and/or denatured from the functionalized support material, and the amplification reagent with one or more amplification enzymes under conditions effective to amplify the nucleic acid to produce amplicons.

92. A method of amplifying nucleic acid, the method comprising:

providing sample material comprising lysed nucleic acid-containing material that includes nucleic acid;

contacting the nucleic acid-containing material with functionalized support material (preferably, nonspecific functionalized support material) under conditions effective to capture at least a portion of the nucleic acid by the functionalized support material;

providing a device comprising a process array that comprises process chambers defining volumes for containing sample material or portions thereof and a conduit connecting the process chambers;

delivering functionalized support material and captured nucleic acid to a process chamber of the device;

washing the functionalized support material with nucleic acid attached thereto multiple times in the same process chamber using multiple aliquots of one or more wash solutions;

transferring the functionalized support material with nucleic acid attached thereto to a different process chamber through the conduit by rotating the device;

before, during, or after transferring the functionalized support material with nucleic acid attached thereto, contacting it with an amplification reagent, wherein the amplification reagent comprises one or more primers and optionally one or more probes;

optionally heating the functionalized support material with nucleic acid attached thereto at a temperature effective to release and/or denature at least a portion of the captured nucleic acid in the presence of the amplification reagent; and

contacting the nucleic acid, which is optionally released and/or denatured from the functionalized support material, and the amplification reagent with one or more amplification enzymes under conditions effective to amplify the nucleic acid to produce amplicons.

93. A method of amplifying nucleic acid, the method comprising:

providing a device comprising a process array that comprises process chambers defining volumes for containing sample material or portions thereof and a conduit connecting the process chambers;

delivering sample material comprising lysed nucleic acid-containing material that includes nucleic acid to a process chamber of the device;

contacting the nucleic acid with functionalized support material (preferably, nonspecific functionalized support material) under conditions effective to capture at least a portion of the nucleic acid;

washing the functionalized support material with nucleic acid attached thereto multiple times in the same process chamber using multiple aliquots of one or more wash solutions;

transferring the functionalized support material with nucleic acid attached thereto to a different process chamber through the conduit by rotating the device;
wherein the nucleic acid contacts an amplification reagent located in the different process chamber;
wherein the amplification reagent comprises one or more primers, optionally one or more probes, and one or more amplification enzymes; and
wherein the one or more amplification enzymes are thermally stable at the temperature effective to release and/or denature the nucleic acid;

heating the functionalized support material with nucleic acid attached thereto at a temperature effective to release and/or denature at least a portion of the nucleic acid in the presence of the amplification reagent; and

providing conditions effective to amplify the released and/or denatured nucleic acid to produce amplicons.

94. A method of amplifying nucleic acid, the method comprising:

providing sample material comprising lysed nucleic acid-containing material that includes nucleic acid;

contacting the nucleic acid-containing material with functionalized support material (preferably, nonspecific functionalized support material) under conditions effective to capture at least a portion of the nucleic acid by the functionalized support material;

providing a device comprising a process array that comprises process chambers defining volumes for containing sample material or portions thereof and a conduit connecting the process chambers;

delivering functionalized support material and captured nucleic acid to a process chamber of the device;

washing the functionalized support material with nucleic acid attached thereto multiple times in the same process chamber using multiple aliquots of one or more wash solutions;

transferring the functionalized support material with nucleic acid attached thereto to a different process chamber through the conduit by rotating the device;
wherein the nucleic acid contacts an amplification reagent located in the different process chamber;
wherein the amplification reagent comprises one or more primers, optionally one or more probes, and one or more amplification enzymes; and
wherein the one or more amplification enzymes are thermally stable at the temperature effective to release and/or denature the nucleic acid;

heating the functionalized support material with nucleic acid attached thereto at a temperature effective to release and/or denature the nucleic acid in the presence of the amplification reagent; and

providing conditions effective to amplify the released and/or denatured nucleic acid to produce amplicons.

Objects and advantages of this invention are further illustrated by the following examples, but the particular materials and amounts thereof recited in these examples, as well as other conditions and details, should not be construed to unduly limit this invention.

SEQUENCE FREE TEXT

| | | |
|---|---|---|
| SEQ ID NO: 1 | CATTGATCGCAACGTTCAATTT | primer |
| SEQ ID NO: 2 | TGGTCTTTCTGCATTCCTGGA | primer |
| SEQ ID NO: 3 | TGGAAGTTAGATTGGGATCATAGCGTCAT | probe |
| SEQ ID NO: 4 | CTGTGAGGTCGGTTGTGCG | primer |
| SEQ ID NO: 5 | TTTGGTCCACCTCGCCA | primer |

### EXAMPLES

### Example 1

### Preparation of Metal-Ion Mediated Magnetic Microparticles

Metal-ion mediated magnetic microparticles, for use as an immobilized-metal support material, were prepared from magnetic particles with surface carboxylic acid groups and with a diameter of about 1 μm (DYNABEADS MYONE Carboxylic Acid from Invitrogen, Carlsbad, Calif., or SERA-MAG Magnetic Particles from Thermo Scientific (known as Seradyn, Indianapolis, Ind.). The carboxylated magnetic microparticles were placed in a tube and washed by attracting them to the wall of the tube using a magnet, removing the liquid by aspiration, replacing the liquid volume with the wash solution, removing the tube from the magnetic field, and agitating the tube to resuspend the microparticles.

Prior to metal-ion treatment, the magnetic microparticles were washed twice with 0.1 M MES buffer, pH 5.5 (containing 0.1% TRITON X-100) and then re-suspended in the same buffer. Following the wash step, 0.2 mL of 0.1 M gallium (III) nitrate, or ferric nitrate or zirconium (IV) nitrate in 0.01 M HCl solution per milligram of magnetic microparticles was added to the magnetic microparticle suspension. The mixture was allowed to shake gently for 1 hour (h) at room temperature and subsequently washed with the above MES buffer to remove excess metal ions. The resulting metal-ion mediated magnetic microparticles (Ga(III)-microparticles-1, Fe(III)-microparticles-1, Zr(IV)-microparticles-1, Ga(III)-microparticles-2, Fe(III)-microparticles-2, Zr(IV)-microparticles-2) were resuspended and stored at 4° C. in MES buffer. DYNABEADS MYONE Carboxylic Acid were used to prepare microparticles-1, and SERA-MAG Magnetic Particles were used to prepare microparticles-2.

### Example 2

### Metal Ion Comparison for DNA Capture and Release

In this experiment, 40 μg of Ga(III)-microparticles-1 and 40 μg of Fe(III)-microparticles-1) from Example 1 were used in separate experiments to bind 10⁵ cfu equivalent MRSA DNA (about 1.8 ng) in pH 5.5 MES buffer. The supernatant was designated SNO. The microparticles were then washed with MES buffer twice and each supernatant (designated SN1 and SN2, respectively) was collected. To elute the bound DNA, the microparticles were resuspended in 20 mM sodium phosphate buffer (PO₄, pH 8.5) and heated to 95° C. for 5 minutes. The supernatant (designated SN3) was collected for mecA-FAM RT-PCR analysis.

Five microliters of each sample (SN3) was subjected to real-time PCR amplification for mecA gene using the following optimized concentrations of primers, probe and enzyme, as well as thermo cycles. The sequence of all primers and probes listed below are given in the 5′→3′ orientation and are known and described in Francois, P., et al., Journal of Clinical Microbiology, 2003, Volume 41, 254-260. The forward mecA primer was CATTGATCGCAACGTTCAATTT (SEQ ID NO:1). The mecA reverse primer was TGGTCTTTCTGCATTCCTGGA (SEQ ID NO:2). The mecA probe sequence, TGGAAGTTAGATTGGGATCATAGCGTCAT (SEQ ID NO:3), was dual labeled by 6-carboxyfluorescein (FAM) and IBFQ (IOWA BLACK FQ, Integrated DNA Technologies, Corniville, Iowa) at 5′- and 3′-position, respectively. PCR amplification was performed in a total volume of 10 μL containing 5 μL of sample and 5 μL of the following mixture: two primers (0.5 μL of 10 μM of each), probe (1 μL of 2 μM), MgCl₂ (2 μL of 25 mM) and LightCycler DNA Master Hybridization Probes (1 μL of 10×, Roche, Indianapolis, Ind.). Amplification was performed on the LightCycler 2.0 Real-Time PCR System (Roche) with the following protocol: 95° C. for 30 seconds (denaturation); 45 PCR cycles of 95° C. for 0 seconds (20° C./s slope), 60° C. for 20 seconds (20° C./s slope, single acquisition).

The control samples consisted of DNA (equivalent to the amount used in the binding experiments) suspended in MES and phosphate buffers, respectively. The control DNA samples were not reacted with metal-ion mediated microparticles.

Table 1 shows the mecA PCR analysis data. The high cycle threshold (Ct) values (relative to control samples) in the SN0, SN1, and SN2 samples indicate the quantitative capture of the DNA. The similar Ct values (relative to control samples) in the SN3 samples indicate quantitative release of the captured DNA.

TABLE 1
PCR Analysis Data (The sample was suspended in 100 μL of
buffer and 5 μL of the resulting sample and 5 μL of PCR
Master mixture were used for PCR amplification.) Ct values are
reported from duplicate PCR reactions for each sample. A
“Neg” result indicates that there was no measurable
Ct value in the 45 cycles that were run.

| | |
|---|---|
| Sample | Cₜ |

| | | |
|---|---|---|
| Ga(III)MRSA + MES Wash-SN0 | 34.15 | 34.25 |
| Ga(III)MRSA + MES Wash-SN1 | Neg | Neg |
| Ga(III)MRSA + MES Wash-SN2 | 35.89 | 34.81 |
| Ga(III)MRSA + MES Wash-SN3 (PO₄) | 21.12 | 21.10 |
| Fe(III)MRSA + MES Wash-SN0 | 34.69 | 33.80 |
| Fe(III)MRSA + MES Wash-SN1 | 34.50 | Neg |
| Fe(III)MRSA + MES Wash-SN2 | 33.92 | 34.94 |
| Fe(III)MRSA + MES Wash-SN3 (PO₄) | 21.53 | 21.58 |
| 10⁵ MRSA Control-MES | 20.99 | 21.03 |
| 10⁵ MRSA Control-PO₄ | 20.39 | 20.49 |

### Example 3

### Thermostability of Dried MMLV Reverse Transcriptase in Tubes

This experiment examines the effect of dry heat (95° C. and 60° C.) for five minutes on the activity of MMLV Reverse Transcriptase from the TaqMan One Step RT-PCR Master Mix Reagents Kit.

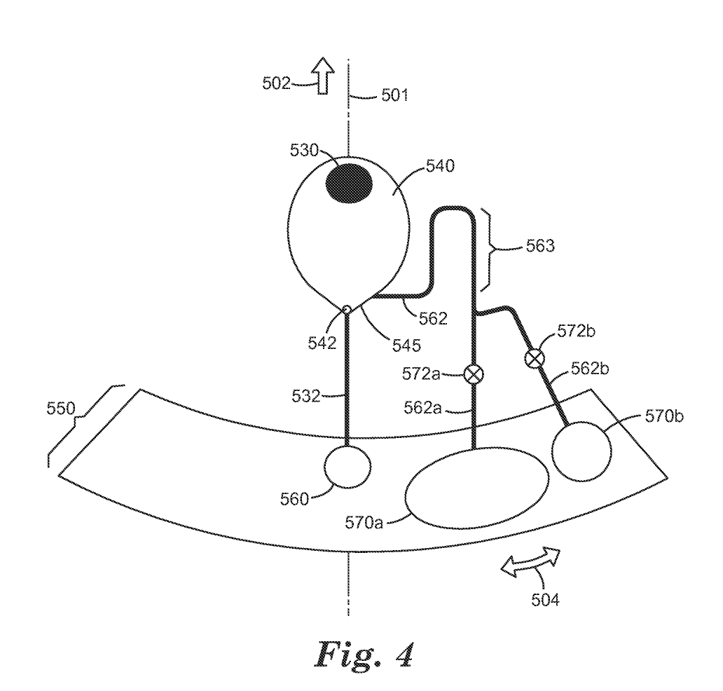

A 1.25-μL aliquot of 40× Multiscribe and RNase Inhibitor Mix (with 50% glycerol content) was dried in a 200-μL PCR tube with continuous HEPA-filtered air flow for 2 h. The dried enzyme in the tube was heated to 95° C. or 60° C. for 5 minutes (min) in Perkin Elmer 9700 GeneAmp, and then resuspended by vortexing with a 48.75-μL reaction mix comprising 25 μL of 2× Universal PCR Master Mix without UNG (ABI), 2.5 μL of 20×TGFβ Gene Expression Assay Mix (ABI), 15 μL of Human Total RNA (50 ng/μL, ABI), and 6.875 μL of RNase-free, DNase-free water (Life Technologies). For each heating treatment, two 20-μL aliquots of the reaction mixture (total volume: 50 μL) were loaded into individual wells of a 96-well plate and placed into a MX3005P (Stratagene) instrument for RT-PCR amplification with an reverse transcription period (48° C. for 30 min), activation of AmpliTaq Gold Enzyme (95° C. for 20 min) and then followed by 45 cycles of PCR (denaturation: 95° C. for 5 sec, annealing/extension: 60° C. for 20 sec). Two 20-μL control reactions were assembled with wet enzymes not subjected to heating. The results are shown in FIG. 5 and Table 2.

TABLE 2
Ct values of TGFβ RT-PCR amplifications
on a MX3005P (Stratagene)

| Reaction | Heating | Ct |
|---|---|---|
| 1 | 95° C.; dry | NA |
| 2 | 95° C.; dry | NA |
| 3 | 60° C.; dry | NA |
| 4 | 60° C.; dry | NA |
| 5 | Wet control: no heat | 30.49 |
| 6 | Wet control: no heat | 30.65 |

### Example 4

### MMLV Reverse Transcriptase Dried and Heated in Microfluidic Discs

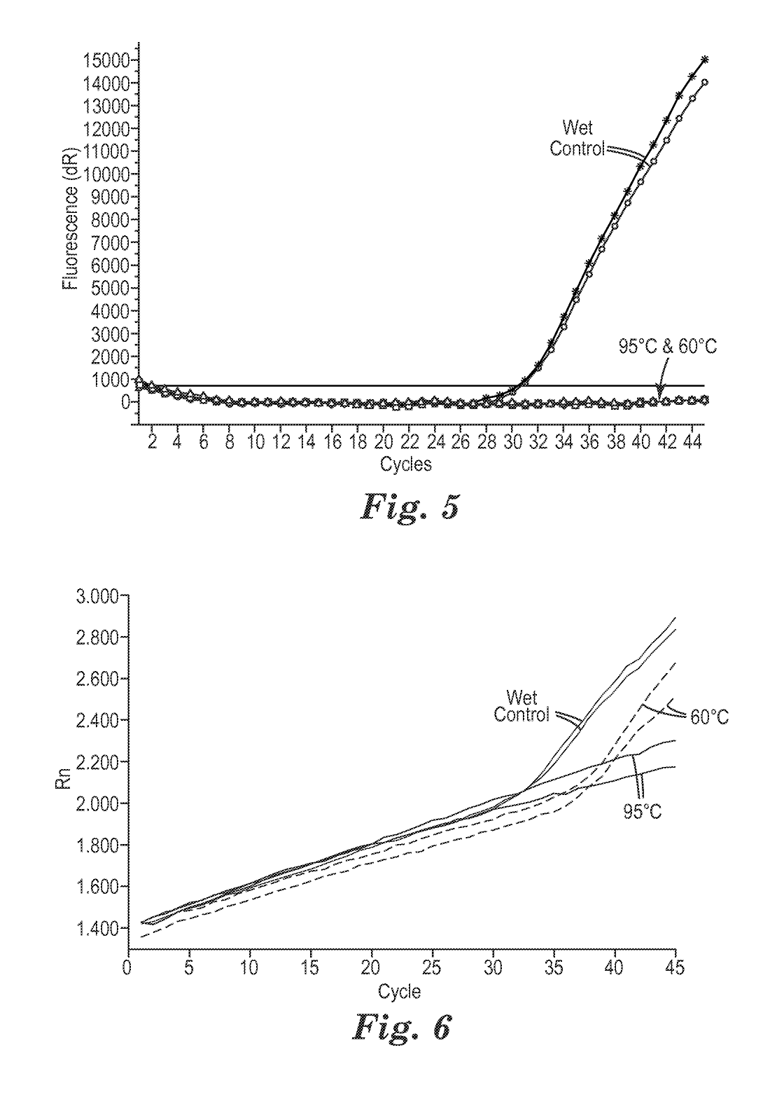

Aliquots (0.75-μL each) of 40× Multiscribe and RNase Inhibitor Mix (with 50% glycerol content) were dried in individual wells of microfluidic discs with continuous HEPA-filtered air flow for 2 h. After drying, the enzymes on the discs were heated to 95° C. or 60° C. for 5 min in an instrument developed by 3M Company (see, e.g., page 43 of U.S. Patent Publication No. 2005/0130177). Each dried enzyme aliquot was resuspended with a 24-μL reaction mix comprising 15-μL of 2× Universal PCR Master Mix without UNG (ABI), 3 μL of 100 mM DTT (Epicentre), 0.75 μL of RNasin (40 units/μL, Promega), and 4.875 μL of RNase-free, DNase-free water (Life Technologies). For each reaction with the heated enzyme, 16 μL of the resuspension (total volume: 24 μL) was mixed with 1 μL of 20×TGFβ Gene Expression Assay Mix (ABI) and 3 μL of Human Total RNA (50 ng/μL, ABI), and then loaded into a well of a 96-well plate and placed into a 7900HT (ABI) instrument for RT-PCR amplification with an reverse transcription period (48° C. for 30 min), activation of AmpliTaq Gold Enzyme (95° C. for 20 min) and then followed by 45 cycles of PCR (denaturation: 95° C. for 5 sec, annealing/extension: 60° C. for 20 sec). Two 20-μL control reactions were assembled with wet enzymes not subjected to heating. The results were shown in FIG. 6 and Table 3.

TABLE 3
Ct values of TGFβ RT-PCR amplifications
on a 7900HT (ABI) instrument

| Reaction | Heating | Ct |
|---|---|---|
| 1 | 95° C.; dry | NA |
| 2 | 95° C.; dry | NA |
| 3 | 60° C.; dry | 37.69 |
| 4 | 60° C.; dry | 38.02 |
| 5 | Wet control: no heat | 31.87 |
| 6 | Wet control: no heat | 32.95 |

The results of the experiments (Examples 3 and 4) indicated that dry heating to 95° C. for 5 min totally abolished enzyme activity of MMLV Reverse Transcriptase. In Example 3 (MMLV RT heated in tubes), heating to 60° C. for 5 min inactivated MMLV Reverse Transcriptase. However, supplementation of DTT (in Example 4) restored partial activity of MMLV Reverse Transcriptase after heating to 60° C. The DTT in the 40× Multiscribe and RNase Inhibitor Mix (ABI) may have been oxidized during heating to 60° C. and thus resulted in the failure in reverse transcription with the 60° C. heated MMLV RT in Example 3. This observation is consistent with some reports about reverse transcription at 55° C. with lower efficiency using MMLV Reverse Transcriptase (New England BioLabs website information). The supplementation of DTT after heating to 95° C. for 5 min did not restore MMLV Reverse Transcriptase activity in Example 4. Ideally, the enzyme and DTT (or other reducing agents for MMLV RT activity) should be protected from exposure to high temperatures when reagents are pre-printed in chambers of an integrated microfluidic disc for molecular diagnostics which performs procedures for sample preparation and amplification/detection using MMLV Reverse Transcriptase.

### Example 5

### Extraction and Detection of Bacterial DNA from Spiked Whole Human Blood

A sample preparation method to extract and isolate bacterial DNA from a whole blood matrix may be useful. In this example, a suspension of whole human blood spiked with methicillin-resistant Staphylococcus aureus ATCC #BAA-43 (MRSA) was simultaneously lysed and captured onto Zr(IV)-microparticles-2. After washing and elution, the eluate from the Zr(IV)-microparticles-2 was compared to a control sample via real-time PCR.

Specifically, MRSA was streaked onto non-selective, tryptic soy agar (TSA) media and incubated at 37° C. for 24 hours. Cell suspension was prepared from fresh growth by dilution in TEP buffer (10 mM Tris-HCl, 1 mM EDTA, pH 8.0 and 0.2% PLURONIC L64 (BASF, Mount Olive, N.J.)) using 0.5 McFarland standard corresponding to 1×10⁸ CFU/mL. Serial dilutions were made to obtain different concentrations of bacterial cells.

One hundred (100) μL of appropriate bacterial dilution was added to aliquots of 900 μL of whole human blood to achieve a 1×10² CFU/mL concentration. Two hundred and fifty (250) μL aliquots of spiked whole blood were separated for further processing. Ten (10) μL of Zr(IV)-microparticles-2 (10 mg/mL) and 40 μL of lysostaphin (250 μg/mL, Sigma) were added to each aliquot of spiked whole blood. The bead mixtures were incubated at room temperature for 10 minutes with gentle vortex.

After incubation, the microparticle mixtures were separated with a magnet and 290 μL of each supernatant was removed and discarded (10 μL carryover volume). The microparticles were then washed three times with 90 μL TEP buffer (continuing with 10 μL carryover volume). After the third wash, 10 μL of 20 mg/mL proteinase K (Qiagen, Valencia, Calif.) and 80 μL 20 mM Phosphate, pH 8.5 buffer were added to each sample (100 μL total volume). The mixture was incubated at 65° C. for 10 minutes and then heated at 95° C. for 10 minutes. The heated microparticle mixtures were then separated with a magnet and each supernatant was collected for mecA real-time PCR as described below.

Separately, pure MRSA culture (without whole blood) was extracted and isolated with Zr(IV)-microparticles-2 using a protocol that otherwise followed that above.

Each sample was subjected to real-time PCR amplification for the mecA gene using the following optimized concentrations of primers, probe and enzyme, and thermocycle protocol. The sequence of all primers and probes listed below are given in the 5′→3′ orientation and are known and described in Francois, P., et al., Journal of Clinical Microbiology, 2003, volume 41, 254-260. The forward mecA primer was CATTGATCGCAACGTTCAATTT (SEQ ID NO:1). The mecA reverse primer was TGGTCTTTCTGCATTCCTGGA (SEQ ID NO:2). The mecA probe sequence, TGGAAGTTAGATTGGGATCATAGCGTCAT (SEQ ID NO:3), was dual labeled by 6-carboxyfluorescein (FAM) and IBFQ (IOWA BLACK FQ, Integrated DNA Technologies, Coralville, Iowa) at 5′- and 3′-position, respectively. PCR amplification was performed in a total volume of 10 μL containing 5 μL of sample and 5 μL of the following mixture: two primers (0.5 μL of 10 μM of each), probe (1 μL of 2 μM), MgCl₂ (2 μL of 25 mM) and LightCycler DNA Master Hybridization Probes (1 μL of 10×, Roche, Indianapolis, Ind.). Amplification was performed on the LightCycler 2.0 Real-Time PCR System (Roche) with the following protocol: 95° C. for 30 seconds (denaturation); 45 PCR cycles of 95° C. for 0 seconds (20° C./s slope), 60° C. for 20 seconds (20° C./s slope, single acquisition).

Results were analyzed using the software provided with the Roche LightCycler 2.0 Real Time PCR System. The primers successfully amplified the mecA gene under the conditions presented in this example as shown in Table 4. The results of this experiment indicate that MRSA in whole blood are captured by Zr(IV)-microparticles-2.

TABLE 4
Real-time PCR detection (mecA gene) of MRSA extracted and
isolated from spiked whole blood samples (in duplicate) using
Zr(IV)-microparticles-2 with a microfluidic mimic protocol.
Ct values are reported in duplicate.

| | |
|---|---|
| Sample | Ct |

| | | |
|---|---|---|
| 2.8 × 10² CFU/mL MRSA in | 33.59 | 31.11 |
| whole blood | 32.52 | 31.26 |
| 3.9 × 10² CFU/mL MRSA | 30.13 | 30.76 |
| (pure culture) | | |
| NTC | Negative | Negative |

### Example 6

### Isolation and Detection of Bacterial DNA from Spiked Canine Feces

A sample preparation method to extract and isolate bacterial DNA from a fecal matrix may be useful. In this example, a suspension of canine feces spiked with vancomycin-resistant Enterococcus faecium ATCC #700221 (VRE) was pre-filtered to remove insoluble debris from the sample. VRE in the resulting eluate was then captured onto Zr(IV)-microparticles-2 and lysed on the solid support. After washing and elution, the eluate from the Zr(IV)-microparticles-2 was compared to control samples via real-time PCR.

Specifically, VRE was streaked onto blood agar media and incubated at 37° C. for 20 hours. Cell suspension was prepared from fresh growth by dilution in TEP buffer (10 mM Tris-HCl, 1 mM EDTA, pH 8.0 and 0.2% PLURONIC L64 (BASF, Mount Olive, N.J.)) using 0.5 McFarland standard corresponding to 1×10⁸ CFU/mL.

One-tenth (0.1) g of canine feces was homogenized in 1 mL of 0.1 M 4-morpholineethanesulfonic acid, pH 5.5 (MES) buffer containing 0.1% TRITON X-100 (Sigma-Aldrich, St. Louis, Mo.) by vortex. Ten (10) μL of 1×10⁸ CFU/mL VRE was spiked into the fecal homogenate. The spiked fecal homogenate was briefly vortexed and then filtered through an EMPORE 6065 Filter Plate (3M, St. Paul, Minn.).

Ten (10) μL of 20 mg/mL proteinase K (Qiagen, Valencia, Calif.) and 10 μL of Zr(IV)-microparticles-2 (10 mg/mL) were added to 80 μL of the filtered fecal homogenate. The microparticle mixture was incubated at 37° C. for 10 minutes with 200 rpm shaking and then further incubated at room temperature for 10 minutes with gentle vortex.

After incubation, the sample was separated using a magnet. The supernatant was removed and 100 μL of TEP buffer was added to the sample. The sample was vortexed briefly and reapplied to the magnet. Supernatant was removed and the sample was resuspended in 80 μL of MES buffer.

Ten (10) μL of 12,500 U/mL mutanolysin (Sigma, St. Louis, Mo.) and 10 μL of 25 mg/mL lysozyme (Sigma, St. Louis, Mo.) were added to the sample. The sample was incubated at 37° C. for 10 minutes with 200 rpm shaking and then further incubated at room temperature for 10 minutes with gentle vortex.

After incubation, the microparticle mixture was separated with a magnet and the supernatant was removed and discarded. The microparticles were then washed twice with 100 μL TEP buffer. After the second wash, the microparticles were resuspended in 100 μL of 20 mM Phosphate, pH 8.5 buffer and heated at 95° C. for 10 minutes. The heated microparticle mixture was then separated with a magnet and the supernatant was collected for vanA real-time PCR as described below.

Separately, pure VRE culture (without feces or filtering) was extracted and isolated with Zr(IV)-microparticles-2 using a protocol that otherwise followed that above. Another pure VRE culture (without feces or filtering) was also extracted and isolated with the MagNA Pure LC system using the MagNA Pure LC DNA Isolation Kit III (Bacteria, Fungi) kit (instrument and reagents obtained from Roche, Indianapolis, Ind.) per manufacturer's instructions. The resultant MagNA Pure isolated DNA was then diluted in MES to an equivalent concentration for comparison to the spiked fecal and pure culture samples.

Primers complementary to the vanA gene of vancomycin-resistant Enterococcus faecium are known and described in Volkmann et al., Journal of Microbiological Methods, 2004, volume 56, page 277-286. The forward primer sequence is 5′ CTGTGAGGTCGGTTGTGCG 3′ (SEQ ID NO:4) and the reverse primer sequence is 5′TTTGGTCCACCTCGCCA 3′ (SEQ ID NO:5).

Polymerase chain reaction (PCR) was performed using the LightCycler FastStart DNA Master SYBR Green I kit (Roche, Indianapolis, Ind.). Fourteen microliters (14 μL) of enzyme was added to one tube of reaction buffer. The enzyme/reaction buffer mixture was vortexed and PCR reactions were created in LightCycler capillaries using the following recipe per reaction: 9 μL PCR-grade H₂0, 1 μL of 10 μM forward primer, 1 μL of 10 μM reverse primer, 4 μL enzyme/reaction buffer mix, and 5 μL sample DNA.

Reactions were placed into the Roche LightCycler 2.0 Real-Time PCR System and the following thermocycle profile was applied to the samples: 95° C. for 10 minutes followed by 45 cycles of the following three steps in order, 95° C. for 10 seconds (20° C./s slope), 50° C. for 10 seconds (20° C./s slope) and 72° C. (20° C./s slope, acquisition) for 30 seconds.

Results were analyzed using the software provided with the Roche LightCycler 2.0 Real Time PCR System. The primers successfully amplified the vanA gene under the conditions presented in this example as shown in Table 5. The results of this experiment indicate that VRE in feces are captured by Zr(IV)-microparticles-2 after a pre-filtration step.

TABLE 5
Real-time PCR detection (vanA gene) of VRE extracted and
isolated from spiked canine fecal samples (in quadruplicate)
using filtration and Zr(IV)-microparticles-2. Ct values are
reported in duplicate.

| | |
|---|---|
| Sample | Ct |

| | | |
|---|---|---|
| 10⁵ CFU/mL VRE in feces, filtered | 28.77 | 29.47 |
| | 30.30 | 28.60 |
| | 27.60 | 27.81 |
| | 27.44 | 26.89 |
| 10⁵ CFU/mL VRE (pure culture) | 22.14 | 23.74 |
| MagNA Pure VRE DNA | 20.62 | 20.99 |
| (gc/mL equivalent to 10⁵ CFU/mL) | | |

### Example 7

### Using Metal Ion Beads for DNA Capture with Thermopipetting Wash

Bacterial cells (MRSA) are first lysed in alkaline lysis buffer (0.1 N NaOH, 5% of lithium lauryl sulfate) for 10 minutes. Subsequently, sufficient amount of metal ion magnetic beads (Ga³⁺) in a neutralization buffer (1M HEPES) are added to the lysis buffer to capture the released DNA. The sample is then dispensed into disc in 300 μL, of each well. The disc is then loaded on an instrument which allows to thermopippetting function. The disc is rotated at high speed and valve is opened through laser to decant about 200 μL of the supernant to waste chamber. The disc is then heated to increase the pressure inside of the device and then cooled down to drain 90% of the supernatant. The process is repeated twice for two washes. After the second wash, a third wash buffer is introduced and again 90% of the supernatant is removed through thermopipetting process. Finally, the beads (SN3) are mixed well and collected and transferred to individual tubes.

The collected samples are subjected to PCR analysis. Five microliters of each sample (SN3) was subjected to real-time PCR amplification for mecA gene using the following optimized concentrations of primers, probe and enzyme, as well as thermo cycles. The sequence of all primers and probes listed below are given in the 5′→3′ orientation and are known and described in Francois, P., et al., Journal of Clinical Microbiology, 2003, volume 41, 254-260. The forward mecA primer is CATTGATCGCAACGTTCAATTT (SEQ ID NO:1). The mecA reverse primer is TGGTCTTTCTGCATTCCTGGA (SEQ ID NO:2). The mecA probe sequence, TGGAAGTTAGATTGGGATCATAGCGTCAT (SEQ ID NO:3), is dual labeled by 6-carboxyfluorescein (FAM) and IBFQ (IOWA BLACK FQ, Integrated DNA Technologies, Corniville, Iowa) at 5′- and 3′-position, respectively. PCR amplification is performed in a total volume of 10 μL containing 5 μL of sample and 5 μL of the following mixture: two primers (0.5 μL of 10 μM of each), probe (1 μL of 2 μM), MgCl₂ (2 μL of 25 mM) and LightCycler DNA Master Hybridization Probes (1 μL of 10×, Roche, Indianapolis, Ind.). Amplification was performed on the LightCycler 2.0 Real-Time PCR System (Roche) with the following protocol: 95° C. for 30 seconds (denaturation); 45 PCR cycles of 95° C. for 0 seconds (20° C./s slope), 60° C. for 20 seconds (20° C./s slope, single acquisition).

The complete disclosures of the patents, patent documents, and publications cited herein are incorporated by reference in their entirety as if each were individually incorporated. Various modifications and alterations to this invention will become apparent to those skilled in the art without departing from the scope of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1-30. (canceled)

31. A method of amplifying nucleic acid, the method comprising:
providing a device comprising a process array that comprises process chambers defining volumes for containing sample material or portions thereof and a conduit connecting the process chambers;
providing sample material comprising lysed nucleic acid-containing material that includes nucleic acid;
contacting the lysed nucleic acid-containing material with a functionalized support material under conditions effective to capture at least a portion of the nucleic acid by the functionalized support material;
prior to or after contacting the lysed nucleic acid-containing material with the functionalized support material, delivering the nucleic acid, optionally attached to the functionalized support material, to a process chamber of the device;
washing the functionalized support material with nucleic acid attached thereto multiple times in the same process chamber using multiple aliquots of one or more wash solutions;
transferring the functionalized support material with nucleic acid attached thereto to a different process chamber through the conduit by rotating the device;
before, during, or after transferring the functionalized support material with nucleic acid attached thereto, contacting it with an amplification reagent, wherein the amplification reagent comprises one or more primers and optionally one or more probes; and
providing conditions effective to amplify the nucleic acid to produce amplicons.

32. The method of claim 31 wherein providing sample material comprising lysed nucleic acid-containing material that includes nucleic acid includes:
delivering sample material comprising nucleic acid-containing material to a process chamber of the device, and
contacting the nucleic acid-containing material with one or more lysing reagents under conditions effective to lyse at least a portion of the nucleic acid-containing material and release nucleic acid.

33. The method of claim 32 wherein contacting the lysed nucleic acid-containing material with a functionalized support material occurs in the same process chamber to which the sample material is delivered.

34. The method of claim 32 wherein contacting the lysed nucleic acid-containing material with a functionalized support material includes contacting the lysed nucleic acid-containing material with a first functionalized support material, and wherein:
prior to delivering sample material to a process chamber of the device, the method comprises:
providing sample material comprising nucleic acid-containing material, and
contacting the nucleic acid-containing material with a second functionalized support material, which may be the same or different than the first functionalized support material used to capture at least a portion of the nucleic acid, under conditions effective to capture at least a portion of the nucleic acid-containing material by the second functionalized support material; and
delivering sample material to a process chamber of the device comprises delivering the second functionalized support material and captured nucleic acid-containing material to a process chamber of the device.

35. The method of claim 32 wherein contacting the lysed nucleic acid-containing material with a functionalized support material includes contacting the lysed nucleic acid-containing material with a first functionalized support material, and wherein:
after delivering sample material to a process chamber of the device, the method comprises:
contacting the nucleic acid-containing material with a second functionalized support material, which may be the same or different than the first functionalized support material used to capture at least a portion of the nucleic acid, under conditions effective to capture at least a portion of the nucleic acid-containing material by the second functionalized support material in the process chamber, and
transferring the second functionalized support material and captured nucleic acid-containing material to a different process chamber; and
contacting the nucleic acid-containing material with one or more lysing reagents comprises contacting the nucleic acid-containing material attached to the second functionalized support material with one or more lysing reagents under conditions effective to lyse at least a portion of the nucleic acid-containing material and release nucleic acid.

36. The method of claim 31 wherein:
providing conditions effective to amplify the nucleic acid to produce amplicons comprises contacting the nucleic acid and the amplification reagent with one or more amplification enzymes under conditions effective to amplify the nucleic acid to produce amplicons.

37. The method of claim 31 wherein:
transferring the functionalized support material with nucleic acid attached thereto to a different process chamber and contacting it with an amplification reagent comprises:
transferring the functionalized support material with nucleic acid attached thereto to a different process chamber through the conduit by rotating the device;
wherein the nucleic acid contacts an amplification reagent located in the different process chamber;
wherein the amplification reagent comprises one or more primers, optionally one or more probes, and one or more amplification enzymes; and
wherein the one or more amplification enzymes are thermally stable at the temperature effective to release and/or denature the nucleic acid; and
prior to or while providing conditions effective to amplify the nucleic acid, the method further comprises:
heating the functionalized support material with nucleic acid attached thereto at a temperature effective to release and/or denature at least a portion of the nucleic acid in the presence of the amplification reagent.

38. The method of claim 31 wherein delivering the nucleic acid to a process chamber of the device comprises delivering functionalized support material and captured nucleic acid to a process chamber of the device.

39. The method of claim 31 wherein delivering the nucleic acid to a process chamber of the device comprises delivering sample material comprising lysed nucleic acid-containing material that includes nucleic acid to a process chamber of the device prior to capturing at least a portion of the nucleic acid by the functionalized support material.

40. A method of amplifying nucleic acid, the method comprising:
providing a device comprising a process array that comprises process chambers defining volumes for containing sample material or portions thereof, a conduit connecting the process chambers, and a thermal transfer structure comprising a thermal drive chamber and a resident fluid;
providing sample material comprising lysed nucleic acid-containing material that includes nucleic acid;
contacting the lysed nucleic acid-containing material with a functionalized support material under conditions effective to capture at least a portion of the nucleic acid by the functionalized support material;
prior to or after contacting the lysed nucleic acid-containing material with the functionalized support material, delivering the nucleic acid, optionally attached to the functionalized support material, to a process chamber of the device;
washing and/or decanting the functionalized support material with nucleic acid attached thereto using the thermal transfer structure, comprising:
optionally adding a wash solution to the process chamber comprising the first functionalized support material with nucleic acid attached thereto;
passing a first portion of the resident fluid through a thermal transfer conduit into the process chamber comprising the functionalized support material with nucleic acid attached thereto by heating at least a portion of the resident fluid in the thermal drive chamber, wherein the thermal transfer conduit connects the thermal drive chamber to the process chamber comprising the functionalized support material with nucleic acid attached thereto, and wherein the volume of the resident fluid within the thermal transfer structure increases to force the first portion of the resident fluid into the process chamber comprising the functionalized support material with nucleic acid attached thereto; and
subsequently cooling the heated resident fluid in the thermal transfer structure, wherein the volume of the resident fluid within the thermal transfer structure decreases such that at least a portion of the first portion of the liquid present in the process chamber comprising the first functionalized support material with nucleic acid attached thereto is drawn into the thermal drive chamber through the thermal transfer conduit;
transferring the functionalized support material with nucleic acid attached thereto to a different process chamber through the conduit by rotating the device;
before, during, or after transferring the functionalized support material with nucleic acid attached thereto, contacting it with an amplification reagent, wherein the amplification reagent comprises one or more primers and optionally one or more probes; and
providing conditions effective to amplify the nucleic acid to produce amplicons.

41. The method of claim 40 wherein providing sample material comprising lysed nucleic acid-containing material that includes nucleic acid includes:
delivering sample material comprising nucleic acid-containing material to a process chamber of the device, and
contacting the nucleic acid-containing material with one or more lysing reagents under conditions effective to lyse at least a portion of the nucleic acid-containing material and release nucleic acid.

42. The method of claim 41, wherein contacting the lysed nucleic acid-containing material with a functionalized support material occurs in the same process chamber to which the sample material is delivered.

43. The method claim 41 wherein contacting the lysed nucleic acid-containing material with a functionalized support material includes contacting the lysed nucleic acid-containing material with a first functionalized support material, and wherein:
prior to delivering sample material to a process chamber of the device, the method comprises:
providing sample material comprising nucleic acid-containing material, and
contacting the nucleic acid-containing material with a second functionalized support material, which may be the same or different than the first functionalized support material used to capture at least a portion of the nucleic acid, under conditions effective to capture at least a portion of the nucleic acid-containing material by the second functionalized support material; and
delivering sample material to a process chamber of the device comprises delivering the second functionalized support material and captured nucleic acid-containing material to a process chamber of the device.

44. The method of claim 41 wherein contacting the lysed nucleic acid-containing material with a functionalized support material includes contacting the lysed nucleic acid-containing material with a first functionalized support material, and wherein:
after delivering sample material to a process chamber of the device, the method comprises:
contacting the nucleic acid-containing material with a second functionalized support material, which may be the same or different than the first functionalized support material used to capture at least a portion of the nucleic acid, under conditions effective to capture at least a portion of the nucleic acid-containing material by the second functionalized support material in the process chamber; and
transferring the second functionalized support material and captured nucleic acid-containing material to a different process chamber; and
contacting the nucleic acid-containing material with one or more lysing reagents comprises contacting the nucleic acid-containing material attached to the second functionalized support material with one or more lysing reagents under conditions effective to lyse at least a portion of the nucleic acid-containing material and release nucleic acid.

45. The method of claim 40 wherein:
providing conditions effective to amplify the nucleic acid to produce amplicons comprises contacting the nucleic acid and the amplification reagent with one or more amplification enzymes under conditions effective to amplify the nucleic acid to produce amplicons.

46. The method of claim 40 wherein:
transferring the functionalized support material with nucleic acid attached thereto to a different process chamber and contacting it with an amplification reagent comprises:
transferring the functionalized support material with nucleic acid attached thereto to a different process chamber through the conduit by rotating the device;
wherein the nucleic acid contacts an amplification reagent located in the different process chamber;
wherein the amplification reagent comprises one or more primers, optionally one or more probes, and one or more amplification enzymes; and
wherein the one or more amplification enzymes are thermally stable at the temperature effective to release and/or denature the nucleic acid; and
prior to or while providing conditions effective to amplify the nucleic acid, the method further comprises:
heating the functionalized support material with nucleic acid attached thereto at a temperature effective to release and/or denature at least a portion of the nucleic acid in the presence of the amplification reagent.

47. The method of claim 40 wherein delivering the nucleic acid to a process chamber of the device comprises delivering functionalized support material and captured nucleic acid to a process chamber of the device.

48. The method of claim 40 wherein delivering the nucleic acid to a process chamber of the device comprises delivering sample material comprising lysed nucleic acid-containing material that includes nucleic acid to a process chamber of the device prior to capturing at least a portion of the nucleic acid by the functionalized support material.

49. A method of amplifying nucleic acid, the method comprising:
providing a device comprising a process array that comprises process chambers defining volumes for containing sample material or portions thereof and a conduit connecting the process chambers;
providing sample material comprising lysed nucleic acid-containing material that includes nucleic acid;
contacting the lysed nucleic acid-containing material with a nonspecific functionalized support material under conditions effective to capture at least a portion of the nucleic acid by the nonspecific functionalized support material, wherein the nonspecific functionalized support material comprises immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or
—P(O)(—OH)₂₋ₓ(—O⁻)ₓ groups; wherein M is selected from the group consisting of zirconium, gallium, iron, aluminum, scandium, titanium, vanadium, yttrium, and a lanthanide; y is an integer from 3 to 6; and x is 1 or 2;
prior to or after contacting the lysed nucleic acid-containing material with the nonspecific functionalized support material, delivering the nucleic acid, optionally attached to the nonspecific functionalized support material, to a process chamber of the device;
washing the nonspecific functionalized support material with nucleic acid attached thereto on the device;
transferring the nonspecific functionalized support material with nucleic acid attached thereto to a different process chamber through the conduit by rotating the device;
before, during, or after transferring the nonspecific functionalized support material with nucleic acid attached thereto, contacting it with an amplification reagent, wherein the amplification reagent comprises one or more primers and optionally one or more probes; and
providing conditions effective to amplify the nucleic acid to produce amplicons.

50. The method of claim 49 wherein providing sample material comprising lysed nucleic acid-containing material that includes nucleic acid includes:
delivering sample material comprising nucleic acid-containing material to a process chamber of the device, and
contacting the nucleic acid-containing material with one or more lysing reagents under conditions effective to lyse at least a portion of the nucleic acid-containing material and release nucleic acid.

51. The method of claim 50 wherein contacting the lysed nucleic acid-containing material with a functionalized support material occurs in the same process chamber to which the sample material is delivered.

52. The method of claim 50 wherein contacting the lysed nucleic acid-containing material with a functionalized support material includes contacting the lysed nucleic acid-containing material with a first functionalized support material, and wherein:
prior to delivering sample material to a process chamber of the device, the method comprises:
providing sample material comprising nucleic acid-containing material; and
contacting the nucleic acid-containing material with a second nonspecific functionalized support material, which may be the same or different than the first nonspecific functionalized support material used to capture at least a portion of the nucleic acid, under conditions effective to capture at least a portion of the nucleic acid-containing material by the second nonspecific functionalized support material; and
delivering sample material to a process chamber of the device comprises delivering the second nonspecific functionalized support material and captured nucleic acid-containing material to a process chamber of the device.

53. The method of claim 50 wherein contacting the lysed nucleic acid-containing material with a functionalized support material includes contacting the lysed nucleic acid-containing material with a first functionalized support material, and wherein:
after delivering sample material to a process chamber of the device, the method comprises:
contacting the nucleic acid-containing material with a second nonspecific functionalized support material, which may be the same or different than the first nonspecific functionalized support material used to capture at least a portion of the nucleic acid, under conditions effective to capture at least a portion of the nucleic acid-containing material by the second nonspecific functionalized support material in the process chamber; and
transferring the second nonspecific functionalized support material and captured nucleic acid-containing material to a different process chamber; and
contacting the nucleic acid-containing material with one or more lysing reagents comprises contacting the nucleic acid-containing material attached to the second nonspecific functionalized support material with one or more lysing reagents under conditions effective to lyse at least a portion of the nucleic acid-containing material and release nucleic acid.

54. The method of claim 49 wherein:
providing conditions effective to amplify the nucleic acid to produce amplicons comprises contacting the nucleic acid and the amplification reagent with one or more amplification enzymes under conditions effective to amplify the nucleic acid to produce amplicons.

55. The method of claim 49 wherein:
transferring the nonspecific functionalized support material with nucleic acid attached thereto to a different process chamber and contacting it with an amplification reagent before, during, or after the transfer, comprises:
transferring the nonspecific functionalized support material with nucleic acid attached thereto to a different process chamber through the conduit by rotating the device;
wherein the nucleic acid contacts an amplification reagent located in the different process chamber;
wherein the amplification reagent comprises one or more primers, optionally one or more probes, and one or more amplification enzymes; and
wherein the one or more amplification enzymes are thermally stable at the temperature effective to release and/or denature the nucleic acid; and
prior to or while providing conditions effective to amplify the nucleic acid, the method further comprises:
heating the nonspecific functionalized support material with nucleic acid attached thereto at a temperature effective to release and/or denature at least a portion of the nucleic acid in the presence of the amplification reagent.

56. The method of claim 49 wherein delivering the nucleic acid to a process chamber of the device comprises delivering nonspecific functionalized support material and captured nucleic acid to a process chamber of the device.

57. The method of claim 49 wherein delivering the nucleic acid to a process chamber of the device comprises delivering sample material comprising lysed nucleic acid-containing material that includes nucleic acid to a process chamber of the device prior to capturing at least a portion of the nucleic acid by the nonspecific functionalized support material.
